(19) United States
(12) Patent Application Publication
(10) Pub. No.: US 2026/0226454 A1
(43) Pub. Date: Aug. 6, 2026

(54) METHODS AND COMPOSITIONS FOR COMPREHENSIVE GENOMIC PROFILING

(71) Applicant: Arima Genomics, Inc., Carlsbad, CA (US)

(72) Inventors: Anthony Schmitt, Holly Springs, NC (US); Siddarth Selvaraj, San Marcos, CA (US)

(73) Assignee: Arima Genomics, Inc., Carlsbad, CA (US)

(21) Appl. No.: 19/150,341

(22) PCT Filed: Jan. 23, 2024
(86) PCT No.: PCT/US2024/012664

§ 371 Date: Jul. 23, 2025

Related U.S. Application Data
(60) Provisional application No. 63/440,886, filed on Jan. 24, 2023.

(51) Int. Cl. C12N 15/10

(52) U.S. Cl. CPC C12N 15/1065

(57) ABSTRACT

The technology relates in part to a method and compositions for comprehensive genomic profiling.

### CROSS REFERENCE TO RELATED APPLICATIONS

This application claims the benefit under 35 U.S.C. § 119(e) of U.S. provisional application No. 63/440,886, filed Jan. 24, 2023, the entire contents of which are incorporated by reference herein.

### BACKGROUND

Identification of tumor drivers can lead to improved therapeutic options and treatment outcomes for cancer patients. Tumor drivers are genetic variants which cause an aberration to cancer gene function or activity. Some tumor drivers are clinically actionable-meaning, the presence of such genetic variants (because of their effect on cancer gene function and/or activity) informs decision and/or actions pertaining to patient management, treatment, or other care. Those decisions and/or actions may include the use of a particular cancer therapy, or provide diagnostic or prognostic information that informs patient management or care in some way. The term “clinically actionable” is known in the art (including but not limited to, for example, Attalla et al, Clin Cancer Research, 2021), and can change over time as new cancer genes, genetic variants, clinical associations, and cancer therapies are discovered.

The types of tumor-driving genetic variants can include single nucleotide variants (SNVs) or Small Insertion or Deletions (InDels) in a cancer gene. Tumor-driving genetic variants can also include copy number variants (CNVs) involving a cancer gene (such as gene amplification or deletion). Tumor-driving genetic variants can also include genomic rearrangements (e.g. translocations, inversions, tandem duplications, large deletions), where the breakpoint of the genomic rearrangement is in a cancer gene. Recently, Applicants described a technology for identifying genomic rearrangements, including any breakpoints occurring either inside of a cancer gene (e.g. those creating a gene-to-gene fusion), or outside of the gene body of the cancer gene yet was shown to affect the function and/or activity of the cancer gene (see, for example, but not limited to WO2023/172882, WO2023/172501, WO2023/172877, WO2023/183706 and PCT/US2023/014608, filed Mar. 6, 2023). Applicants have labeled genomic rearrangements where the breakpoint is outside of the cancer gene as “proximity fusions.”

The current standard of care for the identification of tumor-driving genetic variants for certain cancers is the use of next generation sequencing (NGS). Certain methodologies are well-suited for detecting certain types (but not all types) of tumor driving genetic variants. When these methodologies are all performed, a “comprehensive genomic profile” of the tumor is obtained.

Before Applicants' discoveries, in order to obtain a comprehensive genomic profile of a tumor, two tests were performed on the tumor-Test #1) a (preferably targeted) DNA sequencing test (e.g. such as those commercialized by Foundation Medicine as FOUNDATIONONE™ or by Memorial Sloan Kettering as MSK-IMPACT™), which are primarily used to identify SNV, InDel, and CNV tumor-driving genetic variants, and Test #2a) an (preferably targeted) RNA sequencing test (e.g. such as those sold commercially by Integrated DNA Technologies as ARCHER™ FUSIONPLEX™), which are used to identify gene-to-gene fusions (e.g. a subset of genomic rearrangements whereby the breakpoints are within cancer genes). In light of Applicants' prior technology, currently sold in one manifestation as “Aventa FusionPlus,” genomic rearrangements including gene-to-gene fusions and proximity fusions, a new testing paradigm has emerged in which Test #1 (targeted DNA sequencing) is still performed, followed by Test #2b which is the Applicants' technology. However, because Applicants' technology is relatively new in terms of commercial availability, some testing scenarios would involve 3 total tests-Test #1, Test #2a, and Test #2b. Also of note, current standard of care tumor tests are carried out sequentially, each seeking the detection of certain type(s) of tumor-driving genetic variants. For example, Test #1 (targeted DNA-seq) is completed first to look for SNV, InDel, and CNV tumor-driving genetic variants, and only if no tumor driver is found (for example if no therapeutically targetable tumor-driving genetic variant is found), then Test #2a (RNA-seq) is completed to look for gene-to-gene fusion tumor-driving genetic variants. Standard of care does not yet call for NGS-based testing for proximity fusions using Applicants' technology, and so Applicants' technology is often used today only after Test #1 and #2a were unsuccessful in identifying tumor-driving genetic variants. It is anticipated that Applicants' technology can be used to replace Test #2a (RNA-seq), however even in that scenario, two sequential tests (i.e. Test #1 and Test #2b) must be performed to obtain a comprehensive genomic profile.

In the best-case scenario for a patient, a tumor driver is identified upon the first test (Test #1), because the patient and their care team would be able to make a genetically-informed decision about patient care as soon as possible along the course of the development of their cancer. However, only a paucity of tumor-driving genetic variants are identified using Test #1 (targeted DNA-seq). For example, some reports have indicated that ~10-70% of tumors have an actionable finding upon the first test, and this highly dependent upon the tumor type (Attalla et al, Clin Cancer Research, 2021). So, for a large percentage of patients, they would need at least a second test (e.g. RNA-seq (Test #2a)) as well. The current standard of care is to do these tests sequentially, and each test takes approximately 3-4 weeks (inclusive of pre-testing procedures (e.g. paraffin block sectioning and histological diagnosis), laboratory testing procedures (library generation, sequencing, and informatics), and post-testing procedures (result interpretation, case sign-out by a medical professional, and reporting the results back to the patient. For example, testing labs at elite academic medical centers, such the UCSF Clinical Cancer Genomics Lab, conducts “in-house testing” and reports a 2-3 week turnaround time for their DNA-based UCSF 500 Cancer Gene Panel Test, which does not include the time for pre-testing procedures (which can add another week). A similar turnaround time (a median of <21 days) was estimated in a large (>10,000 sample) institutional study of the MSK-IMPACT DNA sequencing panel (Zehir, Nature Medicine, 2017), and again did not take into account pre-testing procedures in the turnaround time estimate. Therefore, for a patient whose tumor driver is not identified solely on the Test #1, they must wait several weeks for further genomic profiling via Test #2a at a time when several weeks can result in the development of a much more advanced disease state with less favorable clinical outcomes. There is a body of research that has found an association between prolonged time from diagnosis to treatment initiation and inferior patient outcomes across tumor types and stages cancers (e.g. Khorana, PLOS One, 2019 and several other pan-cancer and individual cancer type specific studies known in the art).

And, even beyond that, if the RNA-seq test (Test #2a) also does not identify a driver, patients can use Applicant's technology as a “third-line” test (Test #2b) to search for genomic rearrangements that the RNA-seq test could have missed or proximity fusions that the RNA test is not capable of finding. But, an additional ~4 weeks would have elapsed between the second and third test, and only then has a truly comprehensive genomic profile of their tumor been obtained. In total, a patient may have to wait ~4-8 weeks (depending on the success of the first and/or second tests and which methodology is performed for Test #2) to have the highest chance of identifying their tumor driver. These long timelines are stressful, costly, and detrimental to the patient and their treatment outcome. Clearly, an optimal solution would be one which can identify all types of tumor-driving genetic variants in a first test.

Thus, the methods and compositions described herein provide immense benefit to patients and their treatment outcomes by providing truly comprehensive genetic profiling of tumor tissues much more quickly and with less sample material than is currently possible.

### SUMMARY

Provided in certain aspects is a method for preparing a dual library of template nucleic acid to obtain sequence information from nucleic acid in a sample that includes fragmenting nucleic acid from a sample, thereby producing nucleic acid fragments; adding an affinity purification marker to ends of the nucleic acid fragments; ligating the ends of the nucleic acid fragments; purifying nucleic acid from the sample; separating nucleic acid fragments with the affinity purification marker and nucleic acid fragments without the affinity purification marker; and preparing both the nucleic acid fragments with the affinity purification marker and the nucleic acid fragments without the affinity purification marker for sequencing; thereby creating a dual library of template nucleic acid.

Also provided is a method of analyzing a sample by analyzing sequencing data derived from the dual library to determine the presence or absence of genetic variation in the sample.

Also provided is a method for comprehensive genomic profiling of a sample by analyzing sequencing data derived from the dual library to characterize genetic variation in the sample.

Also provided is a kit with restriction enzyme, label, ligase, substrate capable of binding the label, end-repair reagents, a first set of adaptor oligonucleotides, a second set of adaptor oligonucleotides, a first set of target enrichment oligonucleotides, and a second set of target enrichment oligonucleotides.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 is a schematic of a workflow for comprehensive genomic profiling in accordance with an embodiment of the invention.

FIG. 2, panels A and B, show bar graphs of the analysis of sequence data produced by a workflow in accordance with an embodiment of the invention.

FIG. 3 is a histogram showing the sequencing coverage distribution of the data in FIG. 2.

FIG. 4 is a genome browser snapshot showing the DNA sequencing coverage of the data in FIGS. 2 and 3.

FIG. 5, panels A, B, and C, show contact maps showing DNA interactions from comprehensive genomic profiling in accordance with an embodiment of the invention.

FIG. 6, panels A and B, show a comparison of sequence data from whole genome profiling and that of a workflow for comprehensive genomic profiling in accordance with an embodiment of the invention.

FIG. 7 is a genome browser snapshot of a portion of the data analyzed in FIG. 6.

FIG. 8 is a genome browser snapshot of a portion of the data analyzed in FIG. 6.

FIG. 9 is a genome browser snapshot of a portion of the data analyzed in FIG. 6.

FIG. 10 shows contact maps showing DNA interactions from comprehensive genomic profiling in accordance with an embodiment of the invention.

### APPENDICES

Appendix 1 presents a list of cancer genes containing polynucleotide regions to which oligonucleotide probes can hybridize, and/or to which oligonucleotide probes can be designed to hybridize, in certain implementations. Appendix 1 shows the name of the cancer gene, the chromosome on which the cancer gene is located, the start and end positions of the cancer gene, according to coordinate positions from the Genome Reference Consortium Human Build 38 (GRCH38), and on which Watson (+) or Crick (−) strand the gene is oriented in the sense direction.

Appendix 2 presents a list of cancer genes containing polynucleotide regions to which oligonucleotide probes can hybridize, and/or to which oligonucleotide probes can be designed to hybridize, in certain implementations. Appendix 2 shows the name of the cancer gene, the chromosome on which the cancer gene is located, the start and end positions of the cancer gene (see columns “gene start” and “gene end”), according to coordinate positions from the Genome Reference Consortium Human Build 38 (GRCH38), and on which Watson (+) or Crick (−) strand the gene is oriented in the sense direction.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended. Provided herein are methods and compositions for dual sequencing library preparation, comprehensive genomic profiling, and genetic analyses of biological samples.

### Affinity Purification Markers

The term “affinity purification marker” as used herein, refers to any compound or chemical moiety that is capable of being incorporated within a nucleic acid and can provide a basis for selective purification. For example, an affinity purification marker may include, but not be limited to, a labeled nucleotide linker, a labeled and/or modified nucleotide, nick translation, labeled primer, primer linkers, or tagged linkers. The term “labeled nucleotide linker” as used herein, refers to a type of affinity purification marker comprising any nucleic acid sequence comprising a label that may be incorporated (i.e., for example, ligated) into another nucleic acid sequence. For example, the label may serve to selectively purify the nucleic acid sequence (i.e., for example, by affinity chromatography). Such a label may include, but is not limited to, a biotin label, a histidine label (i.e., 6His), or a FLAG label. The affinity purification marker may be linked to nucleotides or short double stranded DNA adapters. The affinity purification markers may be incorporated into the ends of the fragmented nucleic acid using methods known in the art such as polymerization or ligation using a polymerase or ligase.

### Crosslinking

Methods herein may include contacting a population of cells and/or cell nuclei with one or more crosslinking agents. Crosslinking generally refers to bonding one polymer to another polymer. These bonds may be covalent bonds or ionic bonds. In some applications, crosslinking is used to link DNA within a chromatin complex containing DNA and/or one or more proteins (e.g., histones) to maintain the structure of chromatin complexes. In some applications, crosslinking is used to link proteins with other proteins or polymers (e.g., membrane proteins with other membrane polymers, binding agents, or ligands). Crosslinking may include chemical crosslinking and/or UV crosslinking. Chemical crosslinking may be performed using suitable chemical crosslinking agents known in the art such as an aldehyde (e.g., formaldehyde, glutaraldehyde), disuccinimidyl glutarate (DSG), methanol, ethylene glycol bis(succinimidyl succinate) (EGS), bissulfosuccinimidyl suberate (BS3), 1-Ethyl-3-[3-dimethylaminopropyl]carbodiimide (EDC), formalin, psoralen, aminomethyltrioxsalen, mitomycin C, nitrogen mustard, melphalan, 1,3-butadiene diepoxide, cis diaminedichloroplatinum (II), cyclophosphamide, and the like and combinations thereof.

In some embodiments, nucleic acids present in a cell, a cell nucleus, or a plurality of cells and/or cell nuclei are fixed in position relative to each other by chemical crosslinking, for example by contacting the cells with one or more chemical crosslinkers. This treatment locks in the spatial relationships between portions of nucleic acids in a cell. Any suitable method of fixing the nucleic acids in their positions may be used. In some embodiments, cells and/or cell nuclei are fixed, for example with a fixative, such as an aldehyde, for example formaldehyde or glutaraldehyde. In some embodiments, a sample of one or more cells and/or cell nuclei is crosslinked with a crosslinker to maintain the spatial relationships in the cells/cell nuclei. For example, a sample of cells and/or cell nuclei can be treated with a crosslinker to lock in the spatial information or relationship about the molecules in the cells and/or cell nuclei, such as the DNA and RNA in the cell and/or nucleus. In some embodiments, the relative positions of the nucleic acid can be maintained without using crosslinking agents. For example, nucleic acids may be stabilized using spermine and spermidine. In certain instances, cell nuclei may be stabilized by embedding in a polymer such as agarose. In some embodiments, a crosslinker is a reversible crosslinker. In some embodiments, a crosslinker is reversed, for example after nucleic acid fragments or other polymers are joined. In some embodiments, nucleic acids are released from a crosslinked three-dimensional matrix by treatment with an agent, such as a proteinase, that can degrade proteinaceous material from the sample, thereby releasing the end ligated nucleic acids for further analysis, such as nucleic acid sequencing. A sample may be contacted with a proteinase, such as Proteinase K. In some embodiments, cells and/or cell nuclei are contacted with a crosslinking agent to provide crosslinked cells and/or crosslinked cell nuclei. In some embodiments, cells and/or cell nuclei are contacted with a protein-nucleic acid crosslinking agent, a nucleic acid-nucleic acid crosslinking agent, a protein-protein crosslinking agent, or any combination thereof. Using this method, nucleic acids present in a sample become resistant to spatial rearrangement and the spatial information about the relative locations of nucleic acids in a cell and/or cell nucleus is maintained. In some embodiments, a crosslinker is a reversible crosslinker, such that crosslinked molecules can be easily separated in subsequent steps a method described herein. In some embodiments, a crosslinker is a non-reversible crosslinker, such that crosslinked molecules cannot be easily separated. In some embodiments, a crosslinker is light, such as UV light. In some embodiments, a cross linker is light activated.

### Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), small interfering RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil (U). Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded. Single-stranded DNA (ssDNA), for example, can be generated by denaturing double-stranded DNA by heating or by treatment with alkali, for example. Accordingly, in some embodiments, ssDNA is derived from double-stranded DNA (dsDNA).

Nucleic acid (e.g., genomic DNA, nucleic acid targets, oligonucleotides, probes, primers) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes. When the total number of positions is different between the two nucleotide sequences, gaps may be introduced in the sequence of one or both sequences for optimal alignment. The nucleotides at corresponding positions are then compared, and the percent identity between the two sequences is a function of the number of identical positions shared by the sequences (i.e., % identity= # of identical positions/total # of positions×100). When a position in one sequence is occupied by the same nucleotide as the corresponding position in the other sequence, then the molecules are identical at that position. In certain instances, extra or missing bases within a sequence are expressed as gaps in an alignment and may or may not be factored into a percent identity calculation. For example, a percent identity calculation may include a number of mismatches and gaps or may include a number of mismatches only.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, oligonucleotide, or probe, to a nucleic acid molecule having a sequence complementary to the primer, oligonucleotide, or probe compared to hybridization to a nucleic acid molecule not having a complementary sequence. For example, specific hybridization includes the hybridization of a primer, oligonucleotide, or probe to a target nucleic acid sequence that is complementary to the primer, oligonucleotide, or probe.

Primer, oligonucleotide, or probe sequences and length can affect hybridization to target nucleic acid sequences. Depending on the degree of mismatch between the primer, oligonucleotide, or probe and target nucleic acid, low, medium or high stringency conditions may be used to effect primer/target, oligonucleotide/target, or probe/target annealing. As used herein, the term “stringent conditions” refers to conditions for hybridization and washing. Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2×SSC, 0.1% SDS at 50° C. Another example of stringent hybridization conditions includes hybridization in 6× sodium chloride/sodium citrate (SSC) at about 45° C., followed by one or more washes in 0.2×SSC, 0.1% SDS at 55° C. A further example of stringent hybridization conditions includes hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions can include 0.5 M sodium phosphate, 7% SDS at 65° C., followed by one or more washes at 0.2×SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids.

In some embodiments, target nucleic acids comprise degraded DNA. Degraded DNA may be referred to as low-quality DNA or highly degraded DNA. Degraded DNA may be highly fragmented and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A).

Nucleic acid may be derived from one or more sources (e.g., a biological sample described herein) by methods known in the art. Any suitable method can be used for isolating, extracting and/or purifying DNA from a biological sample (e.g., from blood or a blood product, tissue, tumor), non-limiting examples of which include methods of DNA preparation, various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N. J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, nucleic acid is isolated from a fixed biological sample, e.g., formalin-fixed, paraffin-embedded (FFPE) tissue. Genomic DNA from FFPE tissue may be isolated using commercially available kits-such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In certain aspects, cell-free nucleic acid comprises circulating cancer nucleic acid (e.g., cancer DNA). In certain aspects, cell-free nucleic acid comprises circulating tumor nucleic acid (e.g., tumor DNA).

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In some instances, cancer nucleic acid and/or tumor nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, or tumor nucleic acid).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

### Nucleic Acid Analysis

A method herein may comprise one or more nucleic acid analyses. For example, nucleic acid obtained from a sample from a subject may be analyzed for the presence or absence of a genomic rearrangement. Any suitable process for detecting a genomic rearrangement in a nucleic acid sample may be used. Non-limiting examples of processes for analyzing nucleic acid include amplification (e.g., polymerase chain reaction (PCR)), targeted sequencing, microarray, and fluorescence in situ hybridization (FISH), methods that preserve spatial-proximal contiguity information, methods that preserve spatial-proximity relationships, and methods that generate proximity ligated nucleic acid molecules.

In some embodiments, a nucleic acid analysis comprises nucleic acid amplification. For example, nucleic acids may be amplified under amplification conditions. The term “amplified” or “amplification” or “amplification conditions” generally refer to subjecting a target nucleic acid in a sample to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). Detecting a genomic rearrangement described herein using amplification (e.g., PCR) may include use of primers designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of PCR primers useful for identifying a genomic rearrangement are provided herein.

In some embodiments, a nucleic acid analysis comprises fluorescence in situ hybridization (FISH). Fluorescence in situ hybridization (FISH) is a technique that uses fluorescent probes that bind to a nucleic acid sequence with a high degree of sequence complementarity. In certain configurations, fluorescence microscopy may be used to observe where the fluorescent probe is bound to a chromosome. Detecting a genomic rearrangement described herein using fluorescence in situ hybridization (FISH) may include use of probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of probes useful for identifying a genomic rearrangement are provided herein.

In some embodiments, a nucleic acid analysis comprises a microarray (e.g., a DNA microarray, DNA chip, biochip). A DNA microarray is a collection of DNA probes attached to a solid surface. Probes can be short sections of a gene or other genomic DNA element that can hybridize to target nucleic acids in a sample (e.g., under high-stringency conditions). Probe-target hybridization is usually detected and quantified by detection of fluorophore-, silver-, or chemiluminescence-labeled targets to determine presence, absence, and/or relative abundance of target nucleic acid sequences in the sample. Detecting a genomic rearrangement described herein using DNA microarrays may include use of array probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of array probes useful for identifying a genomic rearrangement are provided herein.

In some embodiments, a nucleic acid analysis comprises sequencing (e.g., genome-wide sequencing, targeted sequencing). For targeted sequencing, a target nucleic acid may be amplified (e.g., by PCR with primers specific to the target), enriched using a probe-based approach, where one or more probes hybridize to a target nucleic acid prior to sequencing, or enriched using Cas9-mediated approaches, such as Cas9-guided adapter ligation, as described in Gilpatrick, T. et al., Targeted nanopore sequencing with Cas9-guided adapter ligation, Nature Biotechnology, volume 38, pages 433-438 (2020). Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., NovaSeq, HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); Element Biosciences (e.g. AVITI), or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides. In some embodiments, a sequencing process generates long sequencing reads or “long reads.” In some embodiments, the nominal, average, mean or absolute length of long reads sometimes is about 1,000 continuous nucleotides to about 100,000 or more contiguous nucleotides. In some embodiments, the nominal, average, mean or absolute length of short reads sometimes is about 5,000 continuous nucleotides to about 500,000 or more contiguous nucleotides. The length of the read is dependent upon the instrument used for sequencing.

In some embodiments, a nucleic acid analysis comprises a method that preserves spatial-proximal relationships and/or spatial-proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi:10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi:10.1038/nrm.2016.104; each of which is incorporated by reference in its entirety, to the extent permitted by law).

Methods that preserve spatial-proximal relationships and/or spatial-proximal contiguity information generally refer to methods that capture and preserve the native spatial conformation exhibited by nucleic acids when associated with proteins as in chromatin and/or as part of a nuclear matrix. Spatial-proximal contiguity information and/or spatial-proximity relationships can be preserved by proximity ligation, by solid substrate-mediated proximity capture (SSPC), by compartmentalization with or without a solid substrate or by use of a Tn5 tetramer. Methods that preserve spatial-proximal contiguity information and/or preserve spatial-proximity relationships may be based on proximity ligation or may be based on a different principle where spatial proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where spatial proximity is inferred based on a principle other than proximity ligation may include, for example, SPRITE, SCSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a nucleic acid analysis comprises generating proximity ligated nucleic acid molecules (e.g., using a method described herein). In some embodiments, a nucleic acid analysis comprises sequencing the proximity ligated nucleic acid molecules, e.g., by a suitable sequencing process known in the art or described herein.

In some embodiments, a nucleic acid analysis comprises a method for preparing nucleic acids from particular types of samples that preserves spatial-proximal contiguity information in the sequence of the nucleic acids. Nucleic acid molecules that preserve spatial-proximal contiguity information can fragmented and sequenced using short-read sequencing methods (e.g., Illumina, nucleic acid fragments of lengths approximately 500 bp) or intact molecules that preserve spatial-proximal contiguity information can be sequenced using long-read sequencing (e.g., Illumina, Oxford Nanopore, or others, nucleic acid fragments of lengths approximately 30 K bp or greater).

In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information and/or spatial-proximity relationships is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface.

In some embodiments, methods that preserve spatial-proximal contiguity information and/or spatial-proximity relationships comprise methods that generate proximity ligated nucleic acid molecules (e.g., using proximity ligation). A proximity ligation method is one in which natively occurring spatially proximal nucleic acid molecules are captured by ligation to generate ligated products. Proximity ligation methods generally capture spatial-proximal contiguity information in the form of ligation products, whereby a ligation junction is formed between two natively spatially proximal nucleic acids. Once the ligation products are formed, the spatial-proximal contiguity information is detected using next generation sequencing, whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules can include a restriction endonuclease, a DNA polymerase, a plurality of nucleotides comprising at least one biotinylated nucleotide, and a ligase. In certain embodiments, two or more restriction endonucleases are used.

A variety of suitable methods for carrying out proximity ligation may be used. For example, one example of a HiC method applied to FFPE tissue samples includes the following steps: (1) fragmentation of chromatin of a solubilized and decompacted FFPE sample with a restriction enzyme (or fragmentation); (2) labelling the digested ends by filling in the 5′-overhangs with biotinylated nucleotides; and (3) ligating the spatially proximal digested ends, thus preserving spatial-proximal contiguity information. Once spatial-proximal contiguity information is preserved, further steps in a HiC method may include: purifying and enriching biotin-labelled ligation junction fragments, preparing a library from the enriched fragments and sequencing the library. Another example of a proximity ligation method may include the following steps: (1) digestion of chromatin with a restriction enzyme (or fragmentation); (2) ligating a labeled nucleotide linker to the fragmented ends; and (3) ligating the spatially proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus).

For methods that include target enrichment, a further step is included where nucleic acids containing target sequences are enriched using one or more capture probes (see e.g., International Patent Application Publication No. WO 2014/168575). A capture probe generally comprises a short sequence of nucleotides or oligonucleotide (e.g., 10-500 bases in length) capable of hybridizing to another nucleotide sequence. In some embodiments, a capture probe comprises a label (e.g., a label for selectively purifying specific nucleic acid sequences of interest). Labels are discussed herein and may include, for example, a biotin or digoxigenin label. In some embodiments, capture probes are designed according to a panel of sequences and/or genes of interest (e.g., a panel of cancer genes provided herein as shown in Appendix 1 or Appendix 2).

In some embodiments, a method herein comprises contacting nucleic acid molecules with a plurality of capture probe species. A plurality of capture probe species may each comprise a polynucleotide identical to or complementary to a subsequence in a gene (e.g., a cancer gene). A plurality of capture probe species may each comprise a polynucleotide identical to or complementary to a subsequence in a subsequence in an exon of a gene (e.g., a cancer gene). A plurality of capture probe species may each comprise a polynucleotide identical to or complementary to a subsequence in an exon of gene (e.g., a cancer gene) listed in Table 1. In some embodiments, a plurality of capture probe species comprises about 10 or more capture probe species. In some embodiments, a plurality of capture probe species comprises about 20 or more capture probe species. In some embodiments, a plurality of capture probe species comprises about 50 or more capture probe species. In some embodiments, a plurality of capture probe species comprises about 100 or more capture probe species. In some embodiments, a plurality of capture probe species comprises about 500 or more capture probe species. In some embodiments, a plurality of capture probe species comprises about 1,000 or more capture probe species. In some embodiments, a plurality of capture probe species comprises about 10,000 or more capture probe species. In some embodiments, a plurality of capture probe species comprises about 100,000 or more capture probe species. In some embodiments, a plurality of capture probe species comprises about 300,000 or more capture probe species.

### Cancers

In some embodiments, a subject from which a sample derives has, or is suspected of having, a disease. In some embodiments, a subject from which a sample derives has, or is suspected of having, cancer. In some embodiments, a subject from which a sample derives has, or is suspected of having, a cancer associated with one or more genetic anomalies described herein. In some embodiments, a subject from which a sample derives has, or is suspected of having, a cancer associated with one or more genes and/or cancer genes described herein.

Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like. In some embodiments, a cancer is a rare cancer. In some embodiments, a cancer is glioma. In some embodiments, a cancer is glioblastoma. In some embodiments, a cancer is pediatric glioblastoma. In some embodiments, a cancer is glioblastoma multiforme/anaplastic astrocytoma with piloid features (ANA PA). In some embodiments, a cancer is a sarcoma. In some embodiments, a cancer is leiomyosarcoma (LMS). In some embodiments, a cancer is myxoid leiomyosarcoma. In some embodiments, a cancer is uterine cancer. In some embodiments, a cancer is uterine leiomyosarcoma. In some embodiments, a cancer is uterine myxoid leiomyosarcoma. In some embodiments, a cancer is metastatic high-grade sarcoma, uterine origin. In some embodiments, a cancer is a brain tumor. In some embodiments, a cancer is a benign brain tumor. In some embodiments, a cancer is an astrocytic brain tumor. In some embodiments, a cancer is subependymal giant cell astrocytoma (SEGA). In some embodiments, a cancer is pleomorphic xanthoastrocytoma (PXA). In some embodiments, a cancer is a malignant brain tumor. In some embodiments, a cancer is a bone cancer. In some embodiments, a cancer is chordoma. In some embodiments, a cancer is a central nervous system (CNS) tumor. In some embodiments, a cancer is meningioma. In some embodiments, a cancer is an embryonal tumor. In some embodiments, a cancer is an embryonal central nervous system tumor. In some embodiments, a cancer is embryonal tumors with multilayered rosettes (ETMR). In some embodiments, a cancer is a kidney/renal cancer. In some embodiments, a cancer is a primitive neuroectodermal tumor (PNET). In some embodiments, a cancer is a kidney primitive neuroectodermal tumor (PNET). In some embodiments, a cancer is lymphoma. In some embodiments, a cancer is Burkitt lymphoma. In some embodiments, a cancer is Burkitt lymphoma (human immunodeficiency virus (HIV)+ and/or Epstein-Barr Virus (EBV)+). In some embodiments, a cancer is Hodgkins lymphoma. In some embodiments, a cancer is classic Hodgkins lymphoma. In some embodiments, a cancer is B cell lymphoma. In some embodiments, a cancer is diffuse large B cell lymphoma. In some embodiments, a cancer is a cytoma. In some embodiments, a cancer is plasmacytoma. In some embodiments, a cancer is osseous plasmacytoma. In some embodiments, a cancer is an adenoma. In some embodiments, a cancer is pituitary adenoma.

### Diagnosis and Treatment

In some embodiments, a method herein comprises providing a diagnosis and/or a likelihood of cancer in a subject. A diagnosis and/or likelihood of cancer may be provided when the presence of a genetic variation described herein is detected. In some embodiments, a method herein comprises performing a further test (e.g., biopsy, blood test, imaging, surgery) to confirm a cancer diagnosis.

In some embodiments, a method herein comprises selecting a sample from a subject. In some embodiments, one or more cancer genes in a selected sample are (or were previously) analyzed for one or more genetic variations associated with cancer. Genetic variations associated with cancer may comprise one or more genetic variations chosen from mutations, translocations, inversions, insertions, deletions, duplications, microdeletions, and microduplications, copy number variations, and the like. In some embodiments, one or more cancer genes may be analyzed for the one or more genetic variations associated with cancer according to one or more methods chosen from RNA-Seq (transcriptome analysis), chromosomal karyotyping, FISH panel, microarray, targeted sequencing, cancer NGS panel, and methylation array. In some embodiments, one or more cancer genes comprise no detectable genetic variation associated with cancer (e.g., as analyzed by one or more of the aforementioned methods).

In some embodiments, a selected sample is (or was previously) analyzed for one or more druggable targets. In some embodiments, one or more cancer genes in a selected sample are or were previously analyzed for one or more druggable targets associated with cancer. Druggable targets may include genes and/or cancer genes (i.e., genes and/or cancer genes encoding druggable targets) provided in a database containing druggable targets (e.g., ONCOKB (Memorial Sloan Kettering's Precision Oncology Knowledge Base)). ONCOKB is a precision oncology knowledge base developed at Memorial Sloan Kettering Cancer Center that contains biological and clinical information about genomic alterations in cancer. In some embodiments, druggable targets include genes and/or cancer genes categorized under one or more therapeutic levels, diagnostic levels, and/or prognostic levels (e.g., in the ONCOKB database). In some embodiments, druggable targets include genes and/or cancer genes categorized under therapeutic level 1 (FDA-approved drugs; 43 genes), therapeutic level 2 (standard care; 24 genes), therapeutic level 3 (clinical evidence; 33 genes) and/or therapeutic level R1/R2 (resistance; 11 genes). In some embodiments, druggable targets include genes and/or cancer genes categorized under diagnostic level Dx1 (required for diagnosis; 22 genes) and/or diagnostic level Dx2 (supports diagnosis; 53 genes). In some embodiments, druggable targets include genes and/or cancer genes categorized under prognostic level Px1 (guideline-recognized with well-powered data; 25 genes) and/or prognostic level Px2 (guideline-recognized with limited data; 15 genes).

In some embodiments, a method comprises (a) selecting a sample from a subject, where the selected sample is (or was previously) analyzed for one or more druggable targets by performing a nucleic acid analysis on the selected sample in accordance with an embodiment of the invention. In some embodiments, a method comprises identifying a new druggable target according to the genomic location of the genetic variation (e.g., a druggable target not analyzed in (a) and/or a druggable target not listed in ONCOKB).

In some embodiments, a method comprises (a) selecting a sample from a subject, where the selected sample is (or was previously) analyzed for one or more druggable targets, and no detectable druggable target is (or was) identified; (b) performing a nucleic acid analysis on the selected sample in accordance with embodiments of the method of the invention; and (c) detecting whether a genetic variation is present or absent in the selected sample according to the nucleic acid analysis in (b), and wherein a breakpoint of a genomic rearrangement is not in proximity (linear proximity and/or spatial proximity) to one or more genes and/or cancer genes encoding the one or more druggable targets analyzed in (a). In some embodiments, a method comprises identifying a new druggable target according to the genomic location of the genomic rearrangement (e.g., a druggable target not analyzed in (a) and/or a druggable target not listed in ONCOKB).

The term “in proximity” may refer to spatial proximity and/or linear proximity. Spatial proximity generally refers to 3-dimensional chromatin proximity, which may be assessed according to a method that preserves spatial-proximal relationships, such as a method described herein or any suitable method known in the art. A genomic rearrangement may be located at a position in spatial proximity to a gene and/or cancer gene when a genomic rearrangement and a gene and/or cancer gene (or a fragment thereof) are ligated in a proximity ligation assay or are bound by a common solid phase in a solid substrate-mediated proximity capture (SSPC) assay, for example. Linear proximity generally refers to a linear base-pair distance, which may be assessed according to mapped distances in a reference genome, for example. Linear proximity distance may be provided as a distance between a 5′ or 3′ end of a genomic rearrangement and a 5′ or 3′ end of a gene and/or cancer gene encoding a druggable target.

In some embodiments, a method herein comprises administering a treatment to a subject. A treatment may be administered to a subject when the presence of a genetic variation described herein is detected. Suitable treatments may be determined by a physician and may include one or more modulators (e.g., activators, blockers) of one or more genes, proteins, cancer genes, oncoproteins (proteins encoded by cancer genes), and/or cancer gene-related components associated with a detected genetic variation.

A cancer gene-related component generally refers to one or more components chosen from (i) a cancer gene, including exons, introns, and 5′ (upstream), e.g. promoter regions, or 3′ (downstream) regulatory elements; (ii) transcription products, mRNA, or cDNA; (iii) translation products, protein, gene products, or gene expression products, or homologs of, synthetic versions of, analogs of, receptors of, agonists to receptors of, antagonists to receptors of, upstream pathway regulators of, or downstream pathway targets of translation products, protein, gene products, or gene expression products; and (iv) any component that could be considered by one skilled in the art as a target for a modulator (e.g., activator, blocker, drug, medicament).

A modulator generally refers to an agent that is capable of changing an activity (e.g., change in level and/or nature of an activity) of a component in a system compared to a component's activity under otherwise comparable conditions when the modulator is absent. A modulator herein may refer to an agent that is capable of changing an activity (e.g., change in level and/or nature of an activity) of a gene, protein, cancer gene, oncoprotein, and/or cancer gene-related component in a system compared to a gene's, protein's, cancer gene's, oncoprotein's, and/or cancer gene-related component's activity under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target component of interest. In some embodiments, a modulator interacts indirectly (e.g., directly with an intermediate agent that interacts with the target component) with a target component of interest. In some embodiments, a modulator affects the level of a target component of interest, as one non-limiting example by impacting an upstream signaling pathway associated with the target component of interest. In some embodiments, a modulator affects an activity of a target component of interest without affecting a level of the target component, as one non-limiting example by impacting a downstream signaling pathway associated with the target component of interest. In some embodiments, a modulator affects both level and activity of a target component of interest, such that an observed difference in activity is not entirely explained by or commensurate with an observed difference in level.

The term “modulator of [cancer gene]” or “[cancer gene] modulator” means “modulator of [cancer gene], modulator of [cancer] protein, and/or [cancer gene]-related components” or “[cancer gene], [cancer] protein, and/or [cancer gene]-related components modulator,” respectively, where [cancer gene] can mean any cancer gene identified herein.

In some embodiments, a method herein comprises predicting an outcome of a cancer treatment. An outcome of a cancer treatment may be predicted when the presence of a genetic variation described herein is detected. For example, an outcome of a cancer treatment that includes a gene-specific modulator and/or a cancer gene-specific modulator may be predicted when the presence of a genetic variation associated with the gene and/or cancer gene is detected.

In some embodiments, a sample from a subject is obtained over a plurality of time points. A plurality of time points may include time point over a number of days, weeks, months, and/or years. In some embodiments, a disease state is monitored over a plurality of time points. For example, a method to detect the presence, absence, or amount of a genetic variation described herein may be performed over a plurality of time points to monitor the status of a disease (e.g., a disease (e.g., cancer) associated with the genetic variation detected). In some embodiments, minimal residual disease (MRD) is monitored in a subject. Minimal residual disease (MRD) generally refers to cancer cells remaining after treatment that often cannot be detected by standard scans (e.g., X-ray, mammogram, computerized tomography (CT) scan, bone scan, magnetic resonance imaging (MRI), positron emission tomography (PET) scan, ultrasound) or tests (blood test, tissue biopsy, needle biopsy, liquid biopsy, endoscopic exam). Such cells have the potential to cause a relapse of cancer in a subject. In some embodiments, a method herein comprises detecting a presence of minimal residual disease (MRD) in a subject when a genetic variation described herein is present. In some embodiments, a method herein comprises detecting a presence of minimal residual disease (MRD) in a subject when a genetic variation described herein is present at a detectable level or amount (e.g., detectable by a method described herein). In some embodiments, a method herein comprises detecting an absence of minimal residual disease (MRD) in a subject when a genetic variation described herein is absent. In some embodiments, a method herein comprises detecting an absence of minimal residual disease (MRD) in a subject when a genetic variation described herein is present at an undetectable level or amount (e.g., undetectable by a method described herein). In some embodiments, a method herein comprises detecting an amount of a genetic variation described herein in a sample. A level of minimal residual disease (MRD) in a subject may be determined according to an amount of genomic rearrangement detected in a sample. In some embodiments, a method herein comprises administering a treatment, or continuing to administer a treatment, to the subject when a genetic variation is present. In some embodiments, a method herein comprises stopping a treatment for the subject when a genetic variation is absent.

### Cancer Genes

A genetic variation may be associated with one or more genes. For example, a genetic variation may be associated with one or more cancer genes. A cancer gene is a gene that, when altered, is associated with cancer. Alterations may include mutations, genomic rearrangements, copy number variations, and the like and combinations thereof. Alterations may be located within a gene and/or cancer gene (i.e., intragenic) or outside of/adjacent to a gene and/or cancer gene (i.e., intergenic, extragenic). For genomic rearrangements, the terms “outside of” and “adjacent to,” as used herein in reference to a genomic rearrangement breakpoint being outside of or adjacent to a gene generally means that a breakpoint of a genomic rearrangement is not within the gene. The genomic rearrangement can contain the gene, such as an inversion of the gene, an insertion of the gene, a duplication of the gene, or the like, or can contain a portion of the gene. In certain aspects, the genomic rearrangement may not include the gene, i.e., the genomic rearrangement (the insertion, inversion, duplication) does not contain the gene, or any portion thereof, but the breakpoint of the genomic rearrangement may be adjacent to the gene.

In certain instances, alterations may be located within a different gene. Alterations may be located in a portion of genomic DNA that is proximal to a gene and/or cancer gene (e.g., within a certain linear proximity and/or within a certain spatial proximity). Alterations may affect expression of a gene and/or cancer gene (e.g., increased expression, decreased expression, no expression, constitutive expression). Alterations may affect the function of a protein encoded by the gene and/or cancer gene (e.g., increased function, decreased function, loss-of-function, gain-of-function, constitutive function, change in function). Non-limiting examples of cancer genes are provided in Appendix 1 or Appendix 2.

### Nucleic Acid Library

Details regarding methods for making a nucleic acid library or dual nucleic acid library are described herein. A nucleic acid library generally refers to a plurality of polynucleotide molecules (e.g., a sample of nucleic acids; nucleic acid from a single cell or single nucleus) that are prepared, assembled and/or modified for a specific process, non-limiting examples of which include immobilization on a solid phase (e.g., a solid support, a flow cell, a bead), enrichment, amplification, cloning, detection, and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI), a palindromic sequence, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters. In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation.

In certain embodiments, a ligation-based library preparation method is used (e.g., ILLUMINA TRUSEQ, Illumina, San Diego CA). Ligation-based library preparation methods often make use of an adapter design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, an identifier is incorporated into a nucleic acid library. An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

In some embodiments, a dual nucleic acid library is created. A dual nucleic acid library is where (at least) two nucleic acid libraries are created from a single biological sample. The dual libraries may be physically separate from one another or virtually separate from one another. In cases of “physically separate” dual libraries, the nucleic acid libraries are separated into discrete physical spaces (i.e. compartments) that are barred from intermixing with other compartments. Such a physical compartment might be separate tubes or the well of a microtiter plate. In cases of “virtually separate” dual libraries, each individual library is tagged with a unique barcode sequence and is not physically barred from intermixing with the other library as the other library has its own distinct unique barcode sequence. In this case, the dual libraries are able to physically intermix.

### Nucleic Acid Sequencing

A method herein may comprise sequencing nucleic acid, thereby generating sequence reads. In some embodiments, a method herein comprises sequencing one or more nucleic acid libraries. In some embodiments, a method herein comprises analyzing sequence reads according to a sequence read analysis. In some embodiments, a sequence read analysis comprises identifying spatial-proximity relationship information (e.g., by analyzing the sequences of nucleic acid fragments comprising ligation junctions).

In some embodiments, a sequencing process herein comprises massively parallel sequencing (i.e., nucleic acid molecules are sequenced in a massively parallel fashion, typically within a flow cell). In some embodiments, a sequencing process herein is a shotgun sequencing process. In some embodiments, a sequencing process herein is a locus-specific sequencing process. In some embodiments, a sequencing process herein is a targeted sequencing process. In some embodiments, a sequencing process herein is a non-locus-specific sequencing process. In some embodiments, a sequencing process herein is a non-targeted sequencing process. In some embodiments, a sequencing process herein comprises single-end sequencing. In some embodiments, a sequencing process herein comprises paired-end sequencing.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acids may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); Element Biosciences (e.g. AVITI system); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides. In some embodiments, a sequencing process generates long sequencing reads or “long reads.” In some embodiments, the nominal, average, mean or absolute length of long reads sometimes is about 1,000 continuous nucleotides to about 100,000 or more contiguous nucleotides. In some embodiments, the nominal, average, mean or absolute length of short reads sometimes is about 5,000 continuous nucleotides to about 500,000 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a sequencing process generates a plurality of sequence reads. The plurality of sequence reads may be further processed (e.g., mapped, quantified, normalized). In some embodiments, hundreds, thousands, tens of thousands, hundreds of thousands, millions, tens of millions, hundreds of millions, or billions of sequence reads are generated by a sequencing process described herein. In some embodiments, a sequencing process generates thousands of sequence reads. In some embodiments, a sequencing process generates millions of sequence reads. In some embodiments, a sequencing process generates thousands to millions of sequence reads. In some embodiments, a sequencing process generates between about 100,000 reads to about 1 billion reads. In some embodiments, a sequencing process generates between about 500,000 reads to about 100 million reads. In some embodiments, a sequencing process generates between about 1 million reads to about 10 million reads. For example, a sequencing process may generate about 1 million reads, about 2 million reads, about 3 million reads, about 4 million reads, about 5 million reads, about 6 million reads, about 7 million reads, about 8 million reads, about 9 million reads, about 10 million reads. In some embodiments, a sequencing process generates about 100,000 or more reads. In some embodiments, a sequencing process generates about 500,000 or more reads. In some embodiments, a sequencing process generates about 1 million or more reads. In some embodiments, a sequencing process generates about 5 million or more reads. In some embodiments, a sequencing process generates about 10 million or more reads.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, a sequencing process is performed at about 0.01-fold coverage to about 1-fold coverage. In some embodiments, a sequencing process is performed at about 0.02-fold coverage. In some embodiments, a sequencing process is performed at about 0.05-fold coverage. In some embodiments, a sequencing process is performed at about 0.1-fold coverage. In some embodiments, a sequencing process is performed at about 1-fold coverage to about 30-fold coverage. In some embodiments, a sequencing process is performed at about 5-fold coverage. In some embodiments, a sequencing process is performed at a coverage of at least about 0.01-fold. In some embodiments, a sequencing process is performed at a coverage of at least about 0.1-fold. In some embodiments, a sequencing process is performed at a coverage of at least about 1-fold. In some embodiments, a sequencing process is performed at a coverage of about 0.01-fold or less. In some embodiments, a sequencing process is performed at a coverage of about 0.1-fold or less. In some embodiments, a sequencing process is performed at a coverage of about 1-fold or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 100-fold coverage or more. For example, specific genomic parts may be sequenced at 200-fold, 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, one nucleic acid sample from one cell is sequenced. In certain embodiments, nucleic acids from each of two or more cells are sequenced. In certain embodiments, nucleic acid samples from two or more cells are pooled, and the pool is sequenced. In the latter embodiments, a nucleic acid sample from each cell may be identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and Phix control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In some embodiments a targeted enrichment, amplification and/or sequencing approach is used. Targeted enrichment is described below.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego CA)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), Singular Genomics (e.g., G4 sequencing platform), Element Biosciences (e.g., AVITI™ System), Ultima Genomics (e.g., UG 100™ sequencing platform), SOLID, Roche/454, PACBIO and/or SMRT, Helicos True Single Molecule Sequencing, Ion Torrent and Ion semiconductor-based sequencing (e.g., as developed by Life Technologies), WildFire, 5500, 5500×1 W and/or 5500×1 W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads) is processed prior to, or in conjunction with, an analysis of the sequenced nucleic acid. For example, sequence reads may be processed according to one or more of the following: aligning, mapping, filtering, counting, normalizing, weighting, generating a profile, and the like, and combinations thereof. Certain processing steps may be performed in any order and certain processing steps may be repeated.

Methods herein may further include generating one or more nucleic acid libraries (e.g., one or more sequencing libraries).

### Oligonucleotides

Provided herein are oligonucleotides. Oligonucleotides may be artificially synthesized. Accordingly, provided herein in certain embodiments are synthetic oligonucleotides. An oligonucleotide generally refers to a nucleic acid (e.g., DNA, RNA) polymer that is distinct from a target nucleic acid (e.g., a target nucleic acid comprising one or more genomic rearrangements described herein), and may be referred to as oligos, probes, and/or primers. Oligonucleotides may be short in length (e.g., less than 50 bp, less than 40 bp, less than 30 bp, less than 20 bp, less than 10 bp). In some embodiments, oligonucleotides are between about 10 to about 500 consecutive nucleotides in length. For example, an oligonucleotide may be about 10, 20, 30, 40, 50, 60, 70, 80, 90, 100, 150, 200, 250, 300, 350, 400, 450, or 500 consecutive nucleotides in length.

Oligonucleotides may be designed to hybridize to a region of a sample nucleic acid that is proximal to, adjacent to, and/or spanning a genomic rearrangement described herein, or portion thereof. Oligonucleotides may be designed to hybridize to a portion or portions of a genome that is/are proximal to, adjacent to, overlapping, partially overlapping, or spanning a genomic rearrangement or portion thereof. Oligonucleotides may be designed to hybridize to a region of a sample nucleic acid that comprises a receiving site, a donor site, or a combination of a receiving site and a donor site.

Oligonucleotides may include probes and/or primers useful for detecting presence, absence, or amount of a genomic rearrangement in a nucleic acid sample. Probes and/or primers may be used in conjunction with any suitable nucleic acid analysis (e.g., a nucleic acid analysis method described herein). For example, probes and/or primers may be used in an amplification process (e.g., PCR, quantitative PCR), FISH (e.g., labeled FISH probes, labeled FISH probe pairs (e.g., with fluorophore and quencher)), microarray, nucleic acid capture, nucleic acid enrichment, nucleic acid sequencing, and the like. In some embodiments, oligonucleotides include a capture probe described herein. In some embodiments, oligonucleotides include a plurality of capture probes described herein.

### Spatial-Proximity Relationships

In some embodiments, a method herein comprises a process that preserves spatial-proximity relationships (e.g., spatial-proximal contiguity; spatial-proximal contiguity information; chromosome conformation capture (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi:10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi:10.1038/nrm. 2016.104; each of which is incorporated by reference in its entirety, to the extent permitted by law)).

Methods herein may include contacting a population of cells and/or cell nuclei with one or more agents that preserve spatial-proximity relationships in the nucleic acid of the cells and/or cell nuclei. Agents that preserve spatial-proximity relationships generally refer to agents used in methods that capture and preserve the native spatial conformation exhibited by nucleic acids when associated with proteins as in chromatin and/or as part of a nuclear matrix. Spatial-proximity relationships may be preserved by any suitable method including, but not limited to, proximity ligation, solid substrate-mediated proximity capture (SSPC), compartmentalization with or without a solid substrate, and/or use of a Tn5 tetramer. Methods that preserve spatial-proximity relationships may be based on proximity ligation or may be based on a different principle where spatial proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where spatial proximity is inferred based on a principle other than proximity ligation may include, for example, SPRITE, SCSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a method herein comprises generating proximity ligated nucleic acid molecules (e.g., using a method described herein). In some embodiments, a method herein comprises sequencing the proximity ligated nucleic acid molecules, e.g., by a suitable sequencing process known in the art or described herein. In some embodiments, nucleic acid molecules may be fragmented and sequenced using short-read sequencing methods (e.g., Illumina, nucleic acid fragments of lengths approximately 500 base pairs). In some embodiments, intact nucleic acid molecules can be sequenced using long-read sequencing (e.g., Illumina, Oxford Nanopore, or others, nucleic acid fragments of lengths approximately 30 kilobases or greater).

In some embodiments, methods that preserve spatial-proximity relationships comprise methods that generate proximity ligated nucleic acid molecules (e.g., using proximity ligation). In some embodiments, a method herein comprises contacting the population of cells and/or cell nuclei with one or more reagents that generate proximity ligated nucleic acid molecules. A proximity ligation method is one in which natively occurring spatially proximal nucleic acid molecules are captured by ligation to generate ligated products. Proximity ligation methods generally capture spatial-proximity relationships in the form of ligation products, whereby a ligation junction is formed between two natively spatially proximal nucleic acids. Once the ligation products are formed, the spatial-proximity relationship may be detected using a suitable sequencing method (e.g., next generation sequencing), whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules may include one or more reagents chosen from a restriction endonuclease (i.e., restriction enzyme), a DNA polymerase, a plurality of nucleotides comprising at least one labeled nucleotide (e.g., biotinylated nucleotide), and a ligase. In certain embodiments, two or more restriction endonucleases are used.

### Fragmentation

In some embodiments, nucleic acid is fragmented using one or more methods known in the art including enzymatic fragmentation, chemical fragmentation, or physical fragmentation. In some embodiments, a fragmentation step comprises contacting a cell, cell nuclei and/or nucleic acid with a form of physical fragmentation (such as sonication or other methods known in the art). In some embodiments, a fragmentation step comprises contacting a cell, cell nuclei and/or nucleic acid with a form of chemical fragmentation (such as bleomycin or other methods known in the art). In some embodiments, a fragmentation step comprises contacting a cell, cell nuclei and/or nucleic acid with an enzyme, such as an endonuclease (such as DNase, Benzonase, a restriction enzyme or other methods known in the art) or an endo-exonuclease (such as micrococcal nuclease or other methods known in the art). In some embodiments, a fragmentation comprises contacting a cell, cell nuclei and/or nucleic acid with one or more restriction endonucleases. In some embodiments, a method herein comprises contacting a cell, cell nuclei and/or nucleic acid with two or more restriction endonucleases. Restriction endonucleases may be chosen from type I, II or III restriction endonucleases such as AccI, AciI, AflIII, AluI, Alw44I, ApaI, AsnI, AvaI, AvaII, BamHI, BanII, BclI, BglI, BglII, BlnI, BsmI, BssHII, BstEII, BstUI, CfoI, CIaI, DdeI, DpnI, DpnII, DraI, EcIXI, EcoRI, ECORI, EcoRII, EcoRV, HaeII, HaeII, HhaI, HindII, HindIII, HpaI, HpaII, KpnI, KspI, MaeII, McrBC, MluI, MluNI, MspI, NciI, NcoI, NdeI, NdeII, NheI, NotI, NruI, NsiI, PstI, PvuI, PvuII, RsaI, SacI, SalI, Sau3AI, ScaI, ScrFI, SfiI, SmaI, SpeI, SphI, SspI, StuI, StyI, SwaI, TaqI, XbaI, and XhoI. In some embodiments, a restriction endonuclease is chosen from one or more of MboI, HinfI, MseI and DdeI. In some embodiments, a restriction endonuclease is chosen from one or more of HpyCH4IV, HinfI, HinP1I and MseI. In some embodiments, a restriction endonuclease is NlaIII. In some embodiments, a restriction endonuclease is chosen from one or more of AciI, HinP1I, HpaII, HpyCH4IV, MspI, and TaqI. In some embodiments, a restriction endonuclease is chosen from one or more of BfaI, MseI, and CviQI. In some embodiments, a restriction endonuclease is chosen from one or more of LlaAI, MboI, MgoI, MkrAI, NdeII, NlaII, NmeCI, NphI, Sau3AI, Kzo9I, DpnII, BstMBI, BssMI, and Bsp143I. In some embodiments, a restriction endonuclease is DpnII. In some embodiments, a restriction endonuclease is HinfI. In some embodiments, a restriction endonuclease is chosen from one or both of DpnII and HinfI.

Contacting a cell, cell nuclei and/or nucleic acid with one or more restriction endonucleases typically generates nucleic acid fragments of varying size (i.e., length). In some embodiments, contacting a cell, cell nuclei and/or nucleic acid with one or more restriction endonucleases generates nucleic acid fragments with an average, mean, or median size of about 200 base pairs to about 1000 base pairs. For example, contacting cell, cell nuclei and/or nucleic acid with one or more restriction endonucleases may generate nucleic acid fragments with an average, mean, or median size of about 200 base pairs, 300 base pairs, 400 base pairs, 500 base pairs, 600 base pairs, 700 base pairs, 800 base pairs, 900 base pairs, or 1,000 base pairs. In some embodiments, contacting a cell, cell nuclei and/or nucleic acid with one or more restriction endonucleases generates nucleic acid fragments with an average, mean, or median size of about 800 base pairs.

A cell, cell nuclei and/or nucleic acid may be contacted with one or more restriction endonucleases for a suitable duration of time. For example, a cell, cell nuclei and/or nucleic acid may be contacted with one or more restriction endonucleases for a duration of time suitable to generate a desired product. In some embodiments, a method herein comprises contacting a cell, cell nuclei and/or nucleic acid with one or more restriction endonucleases for about 2 hours or more. In some embodiments, a method herein comprises contacting a cell, cell nuclei and/or nucleic acid with one or more restriction endonucleases for more than 2 hours. For example, a method herein may comprise contacting a cell, cell nuclei and/or nucleic acid with one or more restriction endonucleases for about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, a method herein comprises contacting a cell, cell nuclei and/or nucleic acid nuclei with one or more restriction endonucleases for more than 8 hours. In some embodiments, a method herein comprises contacting a cell, cell nuclei and/or nucleic acid with one or more restriction endonucleases overnight (e.g., about 8-12 hours).

### Ligase

In some embodiments, a method herein comprises contacting a cell, cell nuclei and/or nucleic acid with an agent comprising a ligase activity. Ligase activity may include, for example, blunt-end ligase activity, nick-sealing ligase activity, sticky end ligase activity, circularization ligase activity, cohesive end ligase activity, DNA ligase activity, RNA ligase activity, single-stranded ligase activity, and double-stranded ligase activity. Ligase activity may include ligating a 5′ phosphorylated end of one polynucleotide to a 3′ OH end of another polynucleotide (5′P to 3′OH). Ligase activity may include ligating a 3′ phosphorylated end of one polynucleotide to a 5′ OH end of another polynucleotide (3′P to 5′OH). In some embodiments, a method herein comprises contacting a cell, cell nuclei and/or nucleic acid with a ligase. Suitable reagents (e.g., ligases) and kits for performing ligation reactions are known and available. Ligases that may be used include but are not limited to, T3 ligase, T4 DNA ligase, T7 DNA Ligase, E. coli DNA Ligase, Electro Ligase®, RNA ligases, T4 RNA ligase 1, T4 RNA ligase 2, SplintR® Ligase, RtcB ligase, Taq ligase, and the like and combinations thereof.

### Polymerase

In some embodiments, the method described herein uses one or more polymerases (e.g., DNA polymerases). Any suitable polymerase may be used including, e.g., DNA polymerase I, TAQ DNA polymerase; E. coli DNA polymerase I, large (Klenow) fragment of DNA polymerase I, T4 DNA polymerase, Bacillus stearothermophilus (Bst) DNA polymerase, thermostable DNA polymerases (e.g., from hyperthermophilic marine Archaea), 9°N™ DNA Polymerase (GENBANK accession no. AAA88769.1), THERMINATOR polymerase (9° N™ DNA Polymerase with mutations: D141A, E143A, A485L), and the like. In some embodiments, a strand displacing polymerase is used (e.g., Bst DNA polymerase).

### Labeled Nucleotides

In some embodiments, the method described herein uses one or more labeled nucleotides. A labeled nucleotide can exist as an individually labeled nucleotide or incorporated into a linker/adaptor. A labeled nucleotide may comprise a member of a binding pair. Binding pairs may include, for example, biotin/avidin, biotin/streptavidin, antibody/antigen, antibody/antibody, antibody/antibody fragment, antibody/antibody receptor, antibody/protein A or protein G, hapten/anti-hapten, folic acid/folate binding protein, vitamin B12/intrinsic factor, chemical reactive group/complementary chemical reactive group, digoxigenin moiety/anti-digoxigenin antibody, fluorescein moiety/anti-fluorescein antibody, steroid/steroid-binding protein, operator/repressor, nuclease/nucleotide, lectin/polysaccharide, active compound/active compound receptor, hormone/hormone receptor, enzyme/substrate, oligonucleotide or polynucleotide/its corresponding complement, the like or combinations thereof. In some embodiments, a labeled nucleotide comprises biotin.

In some embodiments, a labeled nucleotide comprises a first member of a binding pair (e.g., biotin); and a second member of a binding pair (e.g., streptavidin) is conjugated to a solid support or substrate. A solid support or substrate can be any physically separable solid to which a member of a binding pair can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, a solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support can be a collection of particles. In some embodiments, the particles can comprise silica, and the silica may comprise silica dioxide. In some embodiments, the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). A member of a binding pair may be linked to a solid support by covalent bonds or by non-covalent interactions and may be linked to a solid support directly or indirectly (e.g., via an intermediary agent such as a spacer molecule or biotin).

There are a variety of suitable methods for carrying out proximity ligation in accordance with embodiments of the invention may be used. For example, in certain embodiments a HiC method can include the following steps: (1) digestion of chromatin with a restriction endonuclease (or fragmentation); (2) labelling the digested ends by filling in the 5′-overhangs with biotinylated nucleotides; and (3) ligating the spatially proximal digested ends, thus preserving spatial-proximity relationships. Once spatial-proximity relationships are preserved, further steps in a HiC method may include: purifying and enriching biotin-labelled ligation junction fragments, preparing a library from the enriched fragments and sequencing the library. In some embodiments, the biotin can be replaced with any junction marker also described herein as an affinity purification marker. Another example of a proximity ligation method may include the following steps: (1) digestion of chromatin with a restriction endonuclease (or fragmentation); (2) ligating a labeled nucleotide linker to the fragmented ends; and (3) ligating the spatially proximal ends, thus preserving spatial-proximity relationships. Once spatial-proximity relationships are preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see e.g., International Patent Application Publication No. WO 2014/168575). A capture probe generally comprises a short sequence of nucleotides or oligonucleotide (e.g., 10-500 bases in length) capable of hybridizing to another nucleotide sequence. In some embodiments, a capture probe comprises a label (e.g., a label for selectively purifying specific nucleic acid sequences of interest). Labels may include, for example, a biotin or digoxigenin label. In some embodiments, capture probes are designed according to a panel of sequences and/or genes of interest.

### Samples

Provided herein are methods and compositions for processing and/or analyzing nucleic acid. Nucleic acid utilized in methods and compositions described herein may be isolated from a sample obtained from a subject (e.g., a test subject). A subject can be any living or non-living organism, including but not limited to a human and a non-human animal. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a human. A subject may be a male or female. A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a subject is an adult patient. In some embodiments, a subject is a pediatric patient.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism. In some embodiments, a nucleic acid sample is isolated or obtained from a cell(s), tissue, organ, and/or the like of an animal (e.g., an animal subject). In some instances, a nucleic acid sample may be obtained as part of a diagnostic analysis.

A sample or test sample may be any specimen that is isolated or obtained from a subject or part thereof (e.g., a human subject, a cancer patient, a tumor). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of tumor or cancer DNA found in a patient's blood, for example, may be performed using, e.g., whole blood, serum, or plasma. Methods for preparing serum or plasma from blood obtained from a subject (e.g., patient; cancer patient) are known. For example, a subject's blood (e.g., patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation.

In some embodiments, a sample is a tissue sample, a cell sample, a blood sample, or a urine sample. In some embodiments, a sample comprises formalin-fixed, paraffin-embedded (FFPE) tissue. In some embodiments, a sample comprises frozen tissue. In some embodiments, a sample comprises peripheral blood. In some embodiments, a sample comprises blood obtained from bone marrow. In some embodiments, a sample comprises cells obtained from urine. In some embodiments, a sample comprises cell-free nucleic acid. In some embodiments, a sample comprises one or more tumor cells. In some embodiments, a sample comprises one or more circulating tumor cells. In some embodiments, a sample comprises a solid tumor. In some embodiments, a sample comprises a blood tumor.

In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information and/or spatial-proximity relationships is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface.

### Targeted Enrichment

A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers. In addition, target enrichment can be carried out using anchored multiplex PCR, in-solution and solid substrate probe hybridization and separation, and CRISPR-based enrichment methods. Target enrichment may also refer to methods that separate target nucleic acids from non-target nucleic acids by depleting non-target nucleic acids.

### Kits

Provided in certain embodiments are kits. The kits may include any components and compositions described herein (e.g., one or more agents that preserve spatial-proximity relationships in nucleic acid of cells and/or cell nuclei, one or more reagents that generate proximity ligated nucleic acid molecules, one or more agents for generating one or more nucleic acid libraries) useful for performing any of the methods described herein, in any suitable combination. Kits may further include any reagents, buffers, or other components useful for carrying out any of the methods described herein. For example, a kit may include one or more of a first crosslinking agent, a second crosslinking agent, one or more endonucleases, a polymerase, one or more labeled nucleotides, a ligase (e.g., T4 DNA ligase), one or more oligonucleotides, a salt solution (e.g., 50 mM to 200 mM NaCl solution), a proteinase (e.g., Proteinase K) and any combination thereof.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for preserving spatial-proximity relationships in nucleic acid of cells and/or cell nuclei, generating proximity ligated nucleic acid molecules and/or generating one or more nucleic acid libraries. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### EXAMPLES

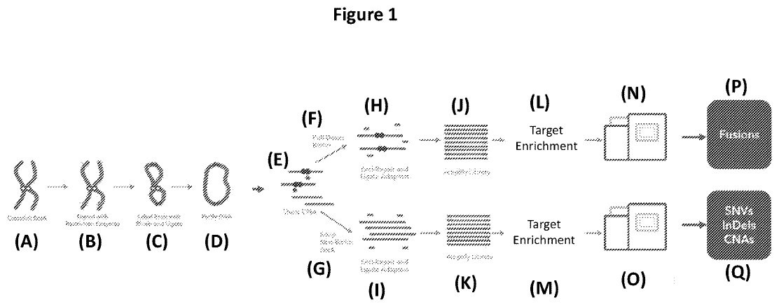

FIG. 1 shows a schematic of a workflow in accordance with one embodiment of the invention. In Step A, a biological sample (here shown as chromatinized DNA) is crosslinked. Depicted are two fragments of nucleic acid that are close in spatial proximity (here shown as a non-limiting example as being within the composition of chromatinized DNA from a biological sample). For example, if the sample is a tumor resection or biopsy from a cancer patient, the sample may be crosslinked using formalin. The formalin crosslinked tissue containing the nucleic acids may be further processed with additional steps including one of more of the following steps known in the art of paraffin embedding, de-waxing, rehydration, lysis, and/or chromatin solubilization/decompaction before proceeding to the next step. See WO 2020106776 as an example. As another example, if the sample is a bone marrow aspirate from a cancer patient, the blood cells (e.g. white blood cells) may be (optionally isolated) crosslinked with formaldehyde and/or other crosslinking agents known in the art. The crosslinked sample may be further processed with additional steps including one of more of the following steps known in the art of lysis, and/or chromatin solubilization/decompaction before proceeding to the next step. Many different sample types, quantities, and formats may be input to crosslinking and may receive sub-subsequent pre-processing steps before proceeding to the nucleic acid fragmenting step. In certain implementations crosslinking may be omitted, if the subsequent Step B and Step C are carried out according to (or similar to) proximity ligation methods in the art that have been reported to not involve crosslinking (Brandt et al, 2016, Exploiting native forces to capture chromosome conformation in mammalian cell nuclei, Mol Syst Biol. 2016 December; 12 (12): 891).

In Step B, nucleic acid from a crosslinked sample is fragmented using one or more methods known in the art including enzymatic fragmentation, chemical fragmentation, or physical fragmentation. The tick marks on the nucleic acid represent sites where the nucleic acid will be fragmented. However, it is also possible that this step may be omitted in the event that the nucleic acid of the crosslinked sample is already sufficiently fragmented. For example, crosslinked nucleic acid within formalin crosslinked tissue (i.e. FFPE tissue) is known to experience fragmentation as a result of the crosslinking, paraffin embedding, and archival process. This fragmentation may make it such that the nucleic acid fragmentation step may be omitted. In certain embodiments, if any steps prior to the intentional nucleic acid fragmentation step result in considerable fragmentation of the nucleic acid, then the fragmentation step of Step B may be omitted.

In step C, ends of the fragmented nucleic acid are labeled with an affinity purification marker capable of subsequent purification (see Step F below). The fragmented nucleic acid ends, at least a portion of which are now labeled with an affinity purification marker, are ligated. Depicted are two fragments of nucleic acid that are close in spatial proximity and have been fragmented and subsequently ligated together. The schematic shows the ligation at both ends, but as with all biological processes, there will never be 100% efficiency and so some nucleic acid fragments may have ends that are not labeled with an affinity purification marker and/or are not ligated. A plurality of the ligation junctions will have an affinity purification marker as depicted in the schematic of FIG. 1.

In Step D, nucleic acid is purified from the other non-nucleic acid components in the sample. Depicted are two fragments of nucleic acid that are close in spatial proximity and have been fragmented, subsequently ligated together and purified away from non-nucleic acid components of the biological sample. This step may also include de-crosslinking prior to nucleic acid purification. De-crosslinking methods and nucleic acid purification methods are known in the art. In some embodiments, this purification can also involve the purification of all nucleic acids, RNA or DNA.

In Step E, the purified ligation products are optionally fragmented. A major reason for Step E is that often the purified ligation products of Step D are too long to be sequenced using current short read sequencing platforms (e.g. Illumina, Element, Singular) and so one must fragment the nucleic acid into smaller pieces of ~400 bp before library preparation and sequencing. However, if the ligation products are already of a length that is compatible with short read sequencing, then this step may be omitted. For example, if the nucleic acid is fragmented with micrococcal nuclease in Step B, it is known in the art that the purified ligation products are around 300 bp and the nucleic acid fragmentation step of Step E can be omitted. Furthermore, the level of DNA fragmentation in FFPE tissues may also be such that the ligation products in FFPE samples may also not require further fragmentation in Step E. The length of the nucleic acid of the purified ligation products can be readily assessed by methods known in the art to help inform whether this step is necessary. It is also noted that while this step may not appear to be necessary, it also would likely not have a detrimental effect if included even if the ligation products are already relatively short. In some implementations, the nucleic acid purification step of Step D and the fragmentation step of Step E maybe carried out in the opposite order. In certain embodiments, after the nucleic acid is fragmented in Step E, it may undergo additional steps not depicted before proceeding to the next step, including but not limited to i) nucleic acid size selection, ii) removal of affinity purification markers from unligated nucleic acid ends, which may be carried out using methods known in the art such as treating the nucleic acid with a polymerase with exonuclease activity (e.g. T4 DNA polymerase) in the presence of nucleotides not linked to an affinity purification marker. Furthermore, before proceeding to Step F or Step G, an affinity purification reagent (e.g. streptavidin coated magnetic beads) capable of binding the affinity purification marker (e.g. biotin labeled nucleotide) is introduced and binds to the affinity purification marker. As illustrated the top 2 nucleic acid fragments of Step E depict nucleic acid fragments resulting from fragmentation of labeled ligation products that have resulted in nucleic acid fragments comprising ligation junctions and therefore comprising affinity purification markers at the ligation junctions and also depicts an affinity purification reagent bound to the affinity purification marker. The bottom 2 nucleic acid fragments of Step E depict nucleic acid fragments resulting from fragmentation of labeled ligation products that have resulted in nucleic acid fragments not comprising ligation junctions and therefore lacking affinity purification markers at the ligation junctions and therefore also lacking affinity purification reagent bound to an affinity purification marker.

In Step F, the (fragmented) ligation products comprising the affinity purification marker are selectively purified. For example, if the affinity purification reagent is streptavidin coated magnetic beads and the affinity purification marker is a biotin labeled nucleotide, then selective purification involves collecting the magnetic beads (which are therefore bound to the streptavidin which is bound to the biotinylated nucleotide of the ligation product) using a magnet, which separates the (fragmented) ligation products comprising the affinity purification marker from those that don't comprise the affinity purification marker.

In Step G, the (fragmented) ligation products not comprising affinity purification marker are retained (rather than discarded as they would be in prior art chromosome conformation capture techniques.

In Step H, the (fragmented) ligation products comprising the affinity purification marker are prepared as a library for sequencing. Depending on the sequencing platform used, this step may entail certain implementations that are sequencing platform specific or sequencing platform agnostic, and are known in the art. For example, for Illumina sequencing platforms, this step involves preparing the fragmented DNA ends (e.g. blunt ending, dA-tailing) and ligating sequencing adapters. Depicted are free floating and ligated sequencing adapters. Step H may occur while the (fragmented) ligation products comprising the affinity purification marker are bound to the affinity purification reagent, as is known in the art.

In Step I, the (fragmented) ligation products not comprising the affinity purification marker are prepared as a library for sequencing. Depending on the sequencing platform used, this step may entail certain implementations that are sequencing platform specific or sequencing platform agnostic, and are known in the art. For example, for Illumina sequencing platforms, this step involves preparing the fragmented DNA ends (e.g. blunt ending, dA-tailing) and ligating sequencing adapters. Depicted are free floating and ligated sequencing adapters. In certain embodiments, the library preparation step may precede the binding of the affinity purification marker with the affinity purification reagents and subsequent selective purification of the (fragmented) ligation products comprising the affinity purification marker and retaining the (fragmented) ligation products not comprising an affinity purification marker. For example, in some embodiments, after nucleic acid fragmentation of Step E, the nucleic acid (which at this time would comprise a mixture of ligation products comprising the affinity purification marker and ligation products not comprising an affinity purification marker) can undergo the steps of library preparation, followed by binding of the affinity purification marker with the affinity purification reagent and subsequent selective purification of the (fragmented) ligation products comprising the affinity purification marker and retaining the (fragmented) ligation products not comprising an affinity purification marker. In certain embodiments, the library preparation step may follow the binding of the affinity purification marker with the affinity purification reagents but precede subsequent selective purification of the (fragmented) ligation products comprising the affinity purification marker and retaining the (fragmented) ligation products not comprising an affinity purification marker. For example, in some embodiments, after nucleic acid fragmentation of Step E, the nucleic acid (which at this time would comprise a mixture of ligation products comprising the affinity purification marker and ligation products not comprising an affinity purification marker can undergo binding of the affinity purification marker with the affinity purification reagent, followed by the steps of library preparation, followed selective purification of the (fragmented) ligation products comprising the affinity purification marker and retaining the (fragmented) ligation products not comprising an affinity purification marker.

In Step J, the sequencing library molecules comprising the (fragmented) ligation products comprising the affinity purification marker are amplified.

In Step K, the sequencing library molecules comprising the (fragmented) ligation products not comprising the affinity purification marker are amplified. In certain embodiments, this step is optional, especially if sufficient nucleic acid is present in Step G or Step I, rendering amplification of Step K unnecessary in certain circumstances. In some embodiments, the outputs of Step H and Step I (as depicted) can be combined prior to amplification. In such an embodiment, the adapters used in Step H and/or Step I contain a barcode that enables the library molecules comprising the (fragmented) ligation products comprising the affinity purification marker to be distinguished from those library molecules comprising the (fragmented) ligation products not comprising the affinity purification marker. In practice this may involve analyzing the barcodes contained in the sequencing reads to determine which sequencing reads are derived from library molecules comprising the (fragmented) ligation products comprising the affinity purification marker and which sequencing reads are derived from library molecules comprising the (fragmented) ligation products not comprising the affinity purification marker.

In Step L, the amplified library molecules originating from the (fragmented) ligation products comprising the affinity purification marker are enriched for a target sequence. In certain embodiments, Step L may be preceded by a step that depletes the template (fragmented) ligation products comprising the affinity purification marker from the output of Step J, such that the molecules used as the input to Step L are the amplified library molecules originating from the (fragmented) ligation products comprising the affinity purification marker, but don't actually contain the affinity purification marker themselves. This implementation is useful when the affinity purification marker of the (fragmented) ligation products is the same as the affinity purification marker of the target enrichment probe. As one example, this implementation is useful when the affinity purification marker of the (fragmented) ligation products is biotin and the affinity purification marker of the target enrichment probe is also biotin.

In Step M, Target enrichment of the amplified library molecules originating from the (fragmented) ligation products not comprising the affinity purification marker. Target enrichment may also be followed by an amplification step. In certain embodiments, if the outputs of Steps H and I (as depicted) are combined, this may result in one (as opposed to two separate) target enrichment steps.

In Step N, the (target enriched) (amplified) library molecules originating from the (fragmented) ligation products comprising the affinity purification marker are sequenced.

In Step O, the (target enriched) (amplified) library molecules originating from the (fragmented) ligation products not comprising the affinity purification marker are sequenced. In certain embodiments, if the outputs of Step H and Step I (as depicted) are combined, this may result in one (as opposed to two separate) sequencing step.

In Step P, the sequence data produced by sequencing the (target enriched) (amplified) library molecules originating from the (fragmented) ligation products comprising the affinity purification marker are analyzed. In certain embodiments, the analysis of the sequence data is preferably for detection of genomic rearrangements, but may also include other variant types. In certain embodiments, analysis of the sequence data is carried out according to other methods known in the art applicable to the analysis of proximity ligation reads, including but not limited to haplotyping phasing, genome sequence assembly, meta-genome sequencing assembly, or combinations thereof and in combination with the detection of genomic rearrangements or other variant types.

In Step Q, the sequence data produced by sequencing the (target enriched) (amplified) library molecules originating from the (fragmented) ligation products not comprising the affinity purification marker is analyzed. In certain embodiments, the analysis of the sequence data is preferably for detection of small variants (e.g. SNVs, InDels) and copy number alterations (CNAs), but may also include other variant types. In certain embodiments, if the outputs of Steps H and I (as depicted) are combined, this may result in one (as opposed to two separate) analysis step. In some embodiments, even if there are two sequencing steps, the analysis step (Step P and Step Q as depicted) can be conducted simultaneously using the sequencing data from both sequencing steps together or separately.

In various embodiments, the depicted workflow may be run simultaneously, or, separately, and to varying degrees of completeness. Using the depicted workflow as an example, one may carry out Steps F, H, J, L, N, and/or P in parallel (simultaneously) with Steps (G, I, K, M, O, and/or Q to detect all variants (e.g. Fusions, SNVs, InDels, CNAs) that can be detected from analysis of sequencing data from library molecules prepared from the (fragmented) ligation products comprising and not comprising the affinity purification marker. Certain implementations may involve first carrying out Steps F, G, I, K, M, O, and/or Q—which in practice refers to the idea of separating the (fragmented) ligation products comprising and not comprising the affinity purification marker, but then first analyzing a biological sample for variants (e.g. SNVs, CNVs, InDels) detected from the library prepared from the (fragmented) ligation products not comprising the affinity purification marker (which would be accomplished from Steps G, I, K, M, O, and/or Q) and then optionally completing Steps H, J, L, N, and/or P to subsequently analyze a biological sample for variants (e.g. fusions) detected from the library prepared from the (fragmented) ligation products comprising the affinity purification marker. This example illustrates how the two depicted workstreams can have some steps completed in parallel (e.g. Step F and Step G), while some steps may be completed sequentially (e.g. first were Steps I, K, M, O, and Q, followed by Steps H, J, L, N, and P). In various other embodiments, many other permutations of which steps are completed in parallel and which steps are completed before others and will depend on the specific application of the technology. As another example, it may be that the workflow is carried out exactly as depicted in FIG. 1, except Step M is omitted. In practice, such an embodiment is useful where one desires to analyze CNAs at a genome-wide scale in Step Q, and also analyze variants (e.g. fusions, as one non-limiting example) associated with specific target genes in Step P. Conversely, in another embodiment, Step L is omitted, which in practice may be used in certain contexts where one desires to analyze variants (e.g. fusions, CNAs) at a genome-wide scale in Step P, and also analyze variants (e.g. SNVs, InDels) associated with specific target genes in Step Q.

Another implementation of the technology may involve adding an affinity purification marker to the nucleic acid fragments of Step G and/or Step I as depicted, and would be a different affinity purification marker than that introduced to label the fragmented ends of in Step C. In certain embodiments, the DNA fragments of Step G and/or Step I are labeled with an affinity purification marker other than biotin, if biotin was used as the marker in Step C. If the nucleic acid fragments of Step F and/or Step H were labeled with a different affinity purification marker than the nucleic acid fragments of Step G and/or Step I, then the nucleic acid fragments of these steps are combined, and in further embodiments, are later separated by using affinity purification reagents that selectively purify the different affinity purification markers.

Another implementation of the technology may involve the preparation of a (targeted) dual library of template nucleic acid from (at least two) paired samples. For example, it is known in the art that certain tumor genetic testing is comprised of preparing libraries, sequencing, and analyzing the sequencing data from a patient's non-tumor circulating blood cells as well as their tumor cells from their tumor biopsy. It is envisioned that such a scenario may involve the preparation of a dual library of template nucleic acid from each sample according to the methods described herein, or, a dual library of template nucleic acid from the tumor cell sample according to the methods described herein but a conventional genomic DNA library from the non-tumor circulating blood cells. In either scenario, the analysis of the resulting data may involve a comparison between the datasets generated from the tumor vs. non-tumor samples, whereby the variants detected in each sample are evaluated as potential tumor-drivers, or, the tumor-specific variants (those not found in the non-tumor sample) are evaluated as potential tumor-drivers.

While it is depicted in FIG. 1, as well as in other variations to the method described herein, that the (fragmented) ligation products comprising the affinity purification marker and the (fragmented) ligation products not comprising the affinity purification marker are physically separated by selective purification, at least temporarily (they may be recombined), prior to amplification step as described above. In other embodiments, other strategies may be employed to eliminate the need for physical separation by selective purification. For example, in certain embodiments, the sample comprising (fragmented) ligation products comprising the affinity purification marker and the (fragmented) ligation products not comprising the affinity purification marker is reacted with an antibody recognizing biotin (which are commercially available and known in the art) following Step (E) of FIG. 1. Then, the sample is reacted with a reagent comprising Protein A conjugated to a transposase, and then reacted in such a way that the transposase inserts adapters into (only) the (fragmented) ligation products comprising the affinity purification marker. This series of steps (antibody binding to the sample, reacting the sample with Protein A conjugated to transposase, and reacting the sample with a transposase such that the transposase inserts an adapter) is similar to the concept described in the “Cut & Tag” method (Kaya-Okur, Nature Communications, 2019), except here the input material is (fragmented) ligation products comprising the affinity purification marker and the (fragmented) ligation products not comprising the affinity purification marker. In certain embodiments, the adapter inserted into (only) the (fragmented) ligation products comprising the affinity purification marker would contain a unique primer annealing sequence. In certain embodiments, a separate set of adapters are added to (only) the (fragmented) ligation products not comprising the affinity purification marker, such as by ligation. These adapters have a unique primer annealing sequence that is different from the adapters added to the (fragmented) ligation products comprising the affinity purification marker. These adapters are leveraged for at least two purposes with respect to delineating the (fragmented) ligation products comprising the affinity purification marker from those (fragmented) ligation products not comprising the affinity purification marker.

First, the amplification step is “multiplex”, with a primer-pair recognizing the unique primer annealing sequence on the adapters of the (fragmented) ligation products comprising the affinity purification marker and a separate primer-pair recognizing the unique primer annealing sequence on the adapters of the (fragmented) ligation products not comprising the affinity purification marker. Each of these primer pairs are conjugated to distinct affinity purification markers (distinct from each other, and distinct from the marker used in the ligation products) such that the amplified library molecules originating from the (fragmented) ligation products comprising the affinity purification marker can be separated from the amplified library molecules originating from the (fragmented) ligation products not comprising the affinity purification marker using affinity purification reagents that selectively purify the different affinity purification markers.

Second, in certain embodiments, alone or in combination with the above, the adapters contain identifier sequences such that during the analysis step, the (target enriched) (amplified) library molecules originating from the (fragmented) ligation products comprising the affinity purification marker are distinguished from the (target enriched) (amplified) library molecules originating from the (fragmented) ligation products not comprising the affinity purification marker.

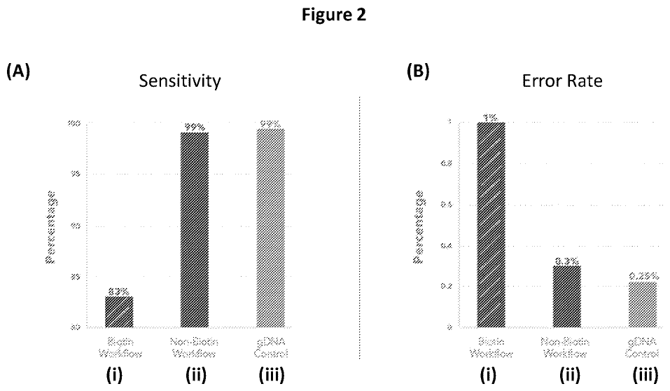

In FIG. 2, analysis of the sequence data produced by sequencing the amplified library molecules originating from the fragmented ligation products not comprising the affinity purification marker demonstrates high performance for the detection of small variants such as single-nucleotide variants (SNVs), whereas analysis of the sequence data produced by sequencing the amplified library molecules originating from the fragmented ligation products comprising the affinity purification marker demonstrates low performance for the detection of small variants such as single-nucleotide variants (SNVs).

HG002 cells (also known as GM24385) were subject to an embodiment of the inventive workflow, as depicted in FIG. 1. More specifically, formaldehyde was used as the crosslinking agent in Step A, a cocktail of DpnII and HinfI were used as the fragmentation enzymes in Step B, a biotin-labeled nucleotide was used as the affinity purification marker in Step C and incorporated into the fragmented ends via polymerization using a DNA polymerase, streptavidin coated magnetic beads were used as the affinity purification reagent in Step E to separate the labeled from unlabeled DNA of Steps F and G, Steps L and M were omitted, deep sequencing was carried out in Steps N and O using an Illumina NovaSeq 6000 instrument, and then the analysis steps of Steps P and Q focused on the analysis of SNVs from the sequence data.

HG002 cells were also subject to a conventional genomic DNA sequencing workflow, comprising only DNA extraction and purification, library preparation, amplification, deep sequencing, and analysis.

Each DNA sequencing dataset was sequenced using 2×150 bp paired end reads to a raw depth ranging from 37×-47× human genome coverage. The raw reads were aligned to the human reference genome using BWA mem, and PCR duplicates were removed using PicardTools. This resulted in between 26×-35× coverage of the human genome of usable uniquely mapped monoclonal (non-duplicate) read-pairs. SNVs were called using the Genome Analysis ToolKit (GATK) HaplotypeCaller.

The “truth” set of SNVs were obtained from a previous analysis of HG002 cells subject to a conventional genomic DNA sequencing workflow, and downloaded from here (ftp-trace.ncbi.nlm.nih.gov/ReferenceSamples/giab/release/), as prepared by the Genome in a Bottle consortia. These truth SNVs were further filtered to contain only those SNVs in high confidence regions. The high confidence SNVs were downloaded from the same genome in a bottle source.

In panel A, a bar plot is shown of the analytical sensitivity. Analytical sensitivity here is defined as the percentage of the true positive variants (from the truth data) that were correctly detected in the respective test DNA sequencing datasets.

Dataset (i), which is labeled as “Biotin Workflow”, is the DNA sequencing dataset from an embodiment of the inventive workflow derived from sequencing the amplified library molecules originating from the fragmented ligation products comprising the affinity purification marker. The sensitivity observed is relatively low, at 83%.

Dataset (ii), which is labeled as “Non-Biotin Workflow”, is the DNA sequencing dataset from an embodiment of the inventive workflow derived from sequencing the amplified library molecules originating from the fragmented ligation products not comprising the affinity purification marker. The sensitivity observed is relatively high, at 99%.

Dataset (iii), which is labeled as “gDNA control”, is the DNA sequencing dataset from the HG002 cells subject to a conventional genomic DNA sequencing workflow, comprising only DNA extraction and purification, library preparation, and amplification. The sensitivity observed is relatively high, at 99%.

In panel B, a bar plot is shown of the error rate. Error rate here is defined as the sum of the SNVs detected in the respective test DNA sequencing datasets that were incorrectly detected relative to the truth dataset, divided by all SNVs detected in the respective test DNA sequencing datasets. SNVs detected in the respective test DNA sequencing datasets that were incorrectly detected relative to the truth dataset could either be a) discordant SNV calls where an SNV is called in the truth dataset and test dataset, but the genomic base at SNV call in the test data is not the same as the genomic base of the SNV in the truth data; or b) a false positive SNV where an SNV is called only in the test dataset (an absent in the truth data).

Dataset (i), which is labeled as “Biotin Workflow”, is the DNA sequencing dataset from an embodiment of the inventive workflow derived from sequencing the amplified library molecules originating from the fragmented ligation products comprising the affinity purification marker. The error rate observed is relatively high, at 1%.

Dataset (ii), which is labeled as “Non-Biotin Workflow”, is the DNA sequencing dataset from an embodiment of the inventive workflow derived from sequencing the amplified library molecules originating from the fragmented ligation products not comprising the affinity purification marker. The error rate observed is relatively low, at 0.3%.

Dataset (iii), which is labeled as “gDNA control”, is the DNA sequencing dataset from the HG002 cells subject to a conventional genomic DNA sequencing workflow, comprising only DNA extraction and purification, library preparation, and amplification. The error rate observed is relatively low, at 0.25%.

Because the “biotin workflow” dataset and “non-biotin workflow” dataset were both generated from the same starting input material (i.e. a single sample source starting with Step A in FIG. 1) and each dataset represents the analysis of a different portion of the DNA molecules when they diverge in the workflow at Steps F & G, this result underscores the significant improvement in small variant (e.g. SNV) detection sensitivity and error rate in the data from the “Non-biotin Workflow” compared to the data from the “Biotin Workflow”. Furthermore, it indicates that the in small variant (e.g. SNV) detection sensitivity and error rate in the data from the “Non-biotin Workflow” is nearly equivalent to the result from the “gDNA control.

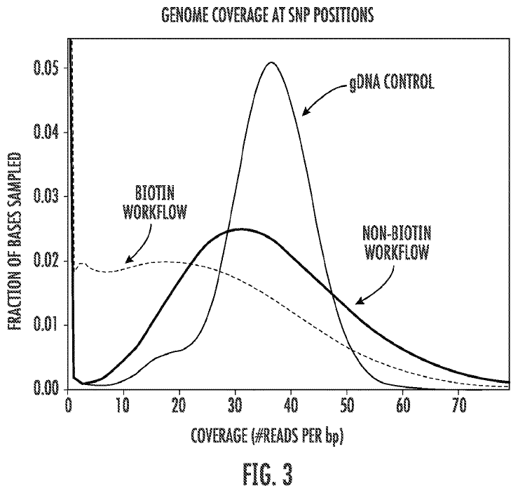

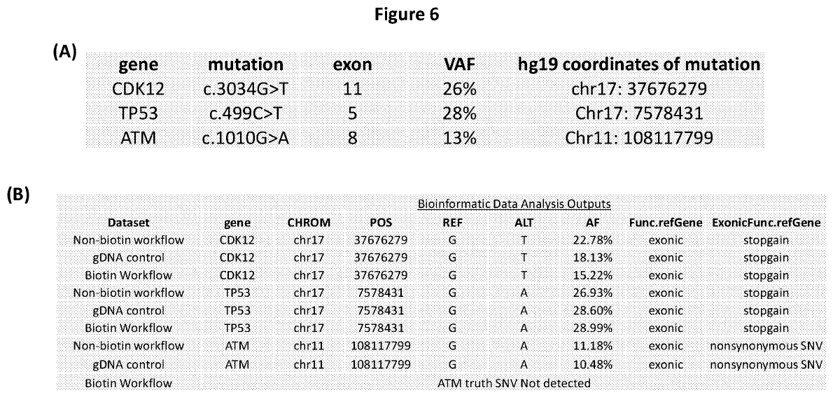

FIG. 3 shows a coverage histogram for the 3 datasets described in FIG. 2. The coverage distribution is widest (least uniform) in the DNA sequencing data which is labeled as “Biotin Workflow”, which is the DNA sequencing dataset from an embodiment of the inventive workflow derived from sequencing the amplified library molecules originating from the fragmented ligation products comprising the affinity purification marker. In contrast, it is also observed that the coverage distribution is substantially more narrow and uniform in the DNA sequencing dataset labeled as “Non-Biotin Workflow”, which is the DNA sequencing dataset from an embodiment of the inventive workflow derived from sequencing the amplified library molecules originating from the fragmented ligation products not comprising the affinity purification marker. Because the “biotin workflow” dataset and “non-biotin workflow” dataset were both generated from the same starting input material (i.e. a single sample source starting with Step A in FIG. 1) and just represent different portion of the DNA molecules when they diverge in the workflow at Steps F & G, this result underscores the significant improvement in coverage uniformity gained from the “non-biotin workflow”. Finally, it is also observed that the most narrow and uniform distribution is in the data which is labeled as “gDNA control”, which is the DNA sequencing dataset from the HG002 cells subject to a conventional genomic DNA sequencing workflow, comprising only DNA extraction and purification, library preparation, and amplification. This suggests that there is room to improve the coverage uniformity in the non-biotin workflow data, such as through changes in the step(s) of the workflow from FIG. 1. However, it is also noted that while the coverage distribution of the non-biotin workflow data isn't as uniform as the gDNA control dataset, that degree of difference in the coverage distribution had little impact on downstream “real-world” analysis applications, such as the sensitive and accurate detection of SNVs (see FIG. 2 as well as FIGS. 6-9.

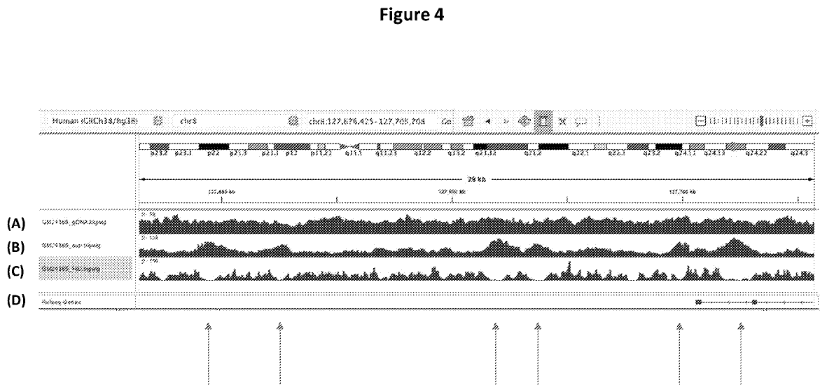

FIG. 4 shows a genome browser snapshot from the IGV software showing the DNA sequencing coverage for the 3 datasets described in FIGS. 2 and 3. Where Row (A) is the DNA sequencing dataset from the HG002 cells subject to a conventional genomic DNA sequencing workflow, comprising only DNA extraction and purification, library preparation, and amplification; Row (B) is the DNA sequencing dataset from an embodiment of the inventive workflow derived from sequencing the amplified library molecules originating from the fragmented ligation products not comprising the affinity purification marker; Row (C) is the DNA sequencing dataset from an embodiment of the inventive workflow derived from sequencing the amplified library molecules originating from the fragmented ligation products comprising the affinity purification marker; and Row (D) is the RefSeq genes. The chromosome coordinates and genome build are shown at the top of the figure.

Similar to FIG. 3, it is observed that the coverage distribution is widest (least uniform-appears “choppy” with valleys of no coverage and peaks of high coverage) in the DNA sequencing data from Row C (“Biotin Workflow” data). In contrast, it is also observed that the coverage distribution is substantially narrower and uniform in the DNA sequencing dataset in Row B (“Non-Biotin Workflow” data). Because the “biotin workflow” dataset and “non-biotin workflow” dataset were both generated from the same starting input material (i.e. a single sample source starting with Step A in FIG. 1) and each dataset represents the analysis of a different portion of the DNA molecules when they diverge in the workflow at Steps F & G, this result underscores the significant improvement in coverage uniformity gained from the “non-biotin workflow”. Finally, it is also observed that the most uniform distribution is in the data which is labeled as Row A (“gDNA control” data). This suggests that there is room to improve the coverage uniformity in the non-biotin workflow data, such as through changes in the step(s) of the workflow from FIG. 1.

However, this analysis also provides insight into what factors may influence coverage uniformity in the “Biotin Workflow” and “Non-biotin workflow” data, at least in the context of the specific embodiment of the inventive workflow carried out to generate these two datasets.

First, it is well known in the art that sequence coverage of HiC datasets (which is essentially equivalent to the “Biotin Workflow” dataset) is strongly biased towards the genomic bases directly adjacent (approximately +/−400 bp) the genomic fragmentation sites. So when using a non-random fragmentation approach, such as done here with the restriction enzymes DpnII and HinfI, you will observe “peaks of coverage around fragmentation sites, a decay in coverage in bases further away from fragmentation sites, and valleys of no coverage for bases not near fragmentation sites.

What is observed in this browser snapshot in “Non-biotin workflow” data is 6 relative increases of sequence coverage (indicated by arrows below the browser snapshot). These arrows point to “bumps” in sequencing coverage relative to other regions in this snapshot within the “non-biotin workflow” dataset.

It is also observed that these relative increases in coverage in the “non-biotin workflow” dataset correlate with valleys in coverage of the “biotin workflow” dataset.

A potential way to improve the coverage uniformity of the “Non-biotin workflow” data could be to improve the DNA fragmentation density and/or uniformity in Step B of FIG. 1, however, as previously noted the coverage distribution of the non-biotin workflow data isn't as uniform as the gDNA control dataset, that degree of difference in the coverage distribution had little impact on downstream “real-world” analysis applications, such as the sensitive and accurate detection of SNVs (see FIG. 2 as well as FIGS. 6-9.

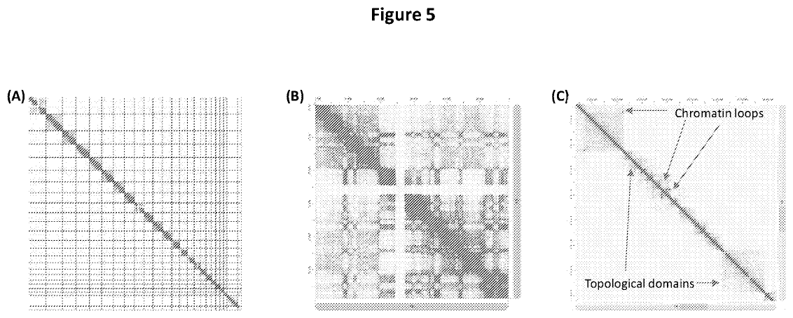

FIG. 5 shows analysis of the dataset from an embodiment of the inventive workflow derived from sequencing the amplified library molecules originating from the fragmented ligation products comprising the affinity purification marker. Analysis demonstrates that the data from this library faithfully captures the 3D genome organization properties across all scales, from chromosome territories, to compartments, to topological domains, to chromatin loops. Because it is well known in the art that proximity ligation data can be used to identify genomic rearrangements (e.g. Dixon et al, Nature Genetics, 2018), this demonstration of high-quality e.g. proximity ligation data (via detection of structural features in the data) indicates that data produced from this would also be optimal for the detection of genomic rearrangements.

Panel (A) is a genome-wide contact map showing the presence of chromosome territories apparent in the data (and also known in the art).

Panel (B) is a chr19 chromosome-wide contact map showing the presence of chromosomal compartments, evident by the checkerboard pattern in the HiC heatmap (and also known in the art). Data is binned at 100 kb resolution.

Panel (C) is a zoom-in view with a chromosomal contact map showing the presence of topological domains and chromatin loops. Zoom-in view shows chromosomal coordinates of approximately chr19:30, 500,000-38, 500,000. Arrows label the areas representing chromatin loops and topological domains.

FIG. 6 shows analysis of the sequence data produced by sequencing the target enriched amplified library molecules originating from the fragmented ligation products not comprising the affinity purification marker demonstrates high performance for the detection of small variants such as single-nucleotide variants (SNVs), whereas analysis of the sequence data produced by sequencing the target enriched amplified library molecules originating from the fragmented ligation products comprising the affinity purification marker demonstrates comparatively lower performance for the detection of small variants such as single-nucleotide variants (SNVs), although still capable of detecting small variants.

A formalin fixed paraffin embedded (FFPE) tumor specimen was subject to an embodiment of the inventive workflow, as depicted in FIG. 1. More specifically, formalin was used as the crosslinking agent in Step A, a cocktail of DpnII and HinfI were used as the fragmentation enzymes in Step B, a biotin-labeled nucleotide was used as the affinity purification marker in Step C and incorporated into the fragmented ends via polymerization using a DNA polymerase, streptavidin coated magnetic beads were used as the affinity purification reagent in Step E to separate the labeled from unlabeled DNA of Steps F and G, Steps L and M were included to perform target enrichment, deep sequencing was carried out in Steps N and O using an Illumina NextSeq instrument, and then the analysis steps of Steps P and Q focused on the analysis of small variants (e.g. SNVs) from the sequence data.

The same formalin fixed paraffin embedded (FFPE) tumor specimen was also subject to a conventional targeted DNA sequencing workflow, comprising only DNA extraction and purification, library preparation, amplification, target enrichment, deep sequencing, and analysis.

The target enrichment method was in-solution probe-based hybridization using the NYU Langone Genome PACT capture probes and capture methodology described in more detail here: accessdata.fda.gov/cdrh_docs/reviews/K202304.pdf

In other embodiments, target enrichment uses oligonucleotides capable of hybridizing to at least one or a plurality of cancer genes in Appendix 1 or Appendix 2.

Each DNA sequencing dataset was processed by aligning the raw reads to the human reference genome (hg19), and PCR duplicates were removed. SNVs were called using LoFreq software, and mutational and functional site annotation of the variants was performed using ANNOVAR software.

The “truth” set of SNVs were obtained from a molecular profiling carried out on the same specimen by the company Caris Life Sciences. These variants, shown in the table of panel A, were reported in the clinical report generated by Caris Life Sciences on this specimen. More specifically, the table of panel A show 3 SNVs, one in each of the genes CDK12, TP53, and ATM. The mutation notation is shown as provided in Caris report, as well as the exon and variant allele frequency as provided in the Caris report. The genomic coordinates of these variants in human reference hg19 is also shown, and was determined by looking up the genomic coordinates of each variant.

The table of panel B summarizes the results of the 3 libraries analyzed and referred to above as the data from “Non-Biotin Workflow”, the “gDNA control”, and the “Biotin Workflow”. The columns indicate the name of the dataset analyzed (“Dataset”), as well as various bioinformatic data analysis outputs from LoFreq and ANNOVAR for each truth variant, if it was detected. If it was detected, then the gene and chromosomal coordinates of the truth variant listed (as reported by the data analysis softwares), the reference allele (“REF”), the detected alternative allele (“ALT”, the estimated allele frequency (“AF”), an annotation of the where the variant occurred within the given gene (“Func.refGene”, for example all of these were exonic SNVs), and functional consequence of the variant in terms of its impact on the codon (“ExonicFunc.refGene”, here they are all non-silent mutations such as “stopgain” which refers to a mutation that causes a premature stop codon, or “nonsynonymous SNV” which just refers to a mutation that causes a different codon and therefore a change in the protein sequence.

The Non-biotin workflow data and gDNA data detect all 3 truth variants, whereas the Biotin Workflow data only detected 2 out of the 3 truth variants.

The allele frequency estimates from analysis of the data from the “Non-Biotin Workflow”, the “gDNA control”, and the “Biotin Workflow” are reasonably close to the estimates from the truth. There are a myriad of reasons why the allele frequency estimates from analysis of the data from the “Non-Biotin Workflow”, the “gDNA control”, and the “Biotin Workflow” wouldn't exact match the truth, such as profiling a different portion of the tumor that may have a slightly different composition of cells, or the fact that the underlying technology (specific DNA extraction method, specific target enrichment method details, sequencing, and analysis software) are different in producing the truth data as compared to the data from the “Non-Biotin Workflow”, the “gDNA control”, and the “Biotin Workflow”. Nonetheless, the allele frequency estimates from analysis of the data from the “Non-Biotin Workflow”, the “gDNA control”, and the “Biotin Workflow” are reasonably close to the estimates from the truth, and it is noted that at least in one case the allele frequency estimate from the “Non-Biotin Workflow” data is considerably closer to matching the allele frequency estimates from the truth data compared to the allele frequency estimates from the “Biotin workflow” data (see CDK12 variant allele frequency estimates).

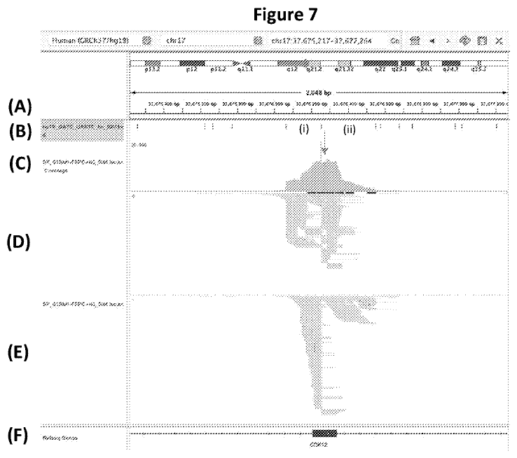

FIG. 7 shows detection of the truth CDK12 SNV from the “Biotin Workflow” dataset generated and analyzed as described in FIG. 6. The genomic coordinates of the region shown in the browser snapshot is shown at the top of the figure. Where Row (A) shows genomic coordinates; Row (B) shows genomic locations of DpnII and HinfI restriction cut site motifs; Row (C) shows read coverage; Row (D) shows a partition of individual reads (shown in the “squished” view settings within IGV) on the forward strand; Row (E) shows a partition of individual reads (shown in the “squished” view settings within IGV) on the reverse strand; and Row (F) shows gene annotation.

A downward pointing arrow vertically spanning rows B and C points to the genomic location of truth SNV.

It is observed there is a considerable amount of sequence coverage bias. One observed source of bias read coverage relative to restriction enzyme cut sites.

For example, the bases with the highest coverage or those directly adjacent to the restriction enzyme cut site that is just left of the downward pointing arrow.

A significant portion of reads appear to all end at the restriction enzyme cut sites. This is apparent when looking at both rows D and E, where the one end of a group of reads all appear to end at the same location.

The sequencing coverage of a given restriction fragment also appears to exhibit strand bias. For example, the 5′ end of the restriction fragment labeled as (i) appears to have significantly skewed coverage bias (enrichment) from reads on the forward strand. A significant portion of reads on the forward strand originate somewhere towards the middle of the restriction fragment (i), and then end at the 5′ end of restriction fragment (i). In contrast, 3′ end of the restriction fragment labeled as (i) appears to have significantly skewed coverage bias (enrichment) from reads on the reverse strand. A significant portion of reads on the reverse strand originate somewhere towards the middle of the restriction fragment (i), and then end at the 3′ end of restriction fragment (i). A similar phenomenon is observed regarding read coverage and restriction fragment labeled as (ii). Again, the 5′ end of the restriction fragment labeled as (ii) appears to have significantly skewed coverage bias (enrichment) from reads on the forward strand. In this case, truth CDK12 SNV is located towards the 5′ end of the restriction fragment labeled as (ii), resulting in strand bias in terms of SNV coverage and more reads comprising the SNV coming from the reads on the positive strand.

It is expected that these biases, alone or in combination, can result in reduced SNV detection performance of one or multiple performance criteria (e.g. sensitivity, error rate, etc).

Although difficult to appreciate in this view, the reads comprising the ALT allele are colored dark gray at the position of the ALT allele in both rows D and E.

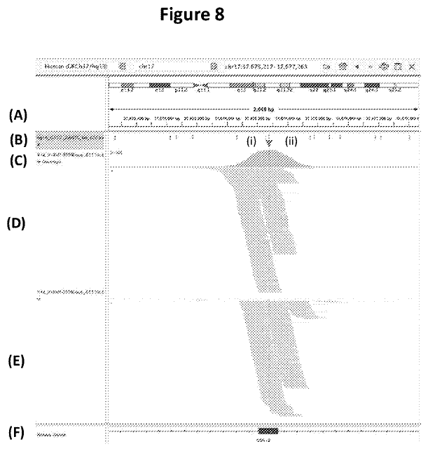

FIG. 8 shows detection of the truth CDK12 SNV from the “Non-Biotin Workflow” dataset generated and analyzed as described in FIG. 6. The genomic coordinates of the region shown in the browser snapshot is shown at the top of the figure. Where Row (A) shows genomic coordinates; Row (B) shows genomic locations of DpnII and HinfI restriction cut site motifs; Row (C) shows read coverage; Row (D) shows a partition of individual reads (shown in the “squished” view settings within IGV) on the forward strand; Row (E) shows a partition of individual reads (shown in the “squished” view settings within IGV) on the reverse strand; and Row (F) shows gene annotation.

A downward pointing arrow in Row B points to the genomic location of truth SNV. It is observed that all the biases apparent in the “Biotin Workflow” data described in FIG. 7 are (expectedly) absent from the data in the “gDNA control” data here. Further, there is a virtually perfect bell-shaped coverage observed in Row (C), which is centered directly on the exon (the exon is the thicker bar just above the text “CDK12” in Row F) which would be expected if there were no coverage biases.

Although difficult to appreciate in this view, the reads comprising the ALT allele are colored dark gray at the position of the ALT allele in both rows D and E.

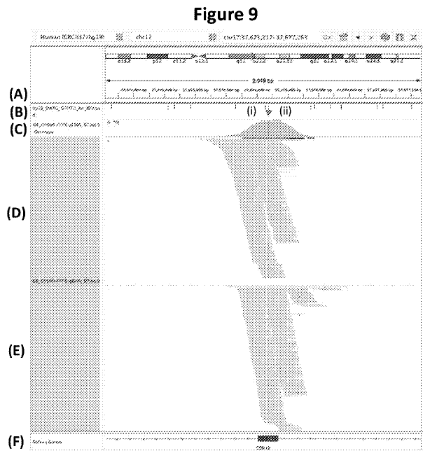

FIG. 9 shows detection of the truth CDK12 SNV from the “gDNA control” dataset generated and analyzed as described in FIG. 6. The genomic coordinates of the region shown in the browser snapshot is shown at the top of the figure. Where Row (A) shows genomic coordinates; Row (B) shows genomic locations of DpnII and HinfI restriction cut site motifs; Row (C) shows read coverage; Row (D) shows partition of individual reads (shown in the “squished” view settings within IGV) on the forward strand; Row (E) shows partition of individual reads (shown in the “squished” view settings within IGV) on the reverse strand; and Row (F) depicts gene annotation.

A downward pointing arrow vertically in row B points to the genomic location of truth SNV. It is observed that all the biases apparent in the “Biotin Workflow” data described in FIG. 7 are (expectedly) absent from the data in the “gDNA control” data here. Further, there is a virtually perfect bell-shaped coverage observed in Row (C), which is centered directly on the exon (the exon is the thicker bar just above the text “CDK12” in Row F) which would be expected if there were no coverage biases.

Although difficult to appreciate in this view, the reads comprising the ALT allele are colored dark gray at the position of the ALT allele in both rows D and E.

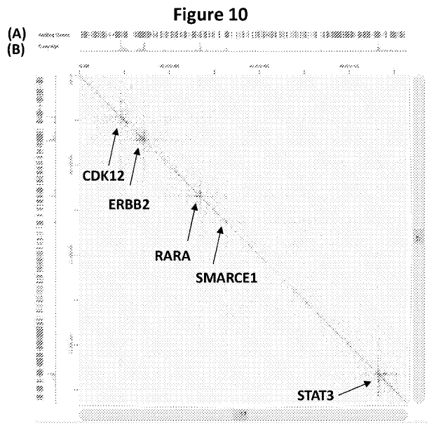

FIG. 10 shows analysis of the dataset generated and analyzed as described in FIG. 6. Analysis of the data derived from sequencing the amplified library molecules originating from the fragmented ligation products comprising the affinity purification marker demonstrates that the data faithfully captures the 3D genome organization properties expected from targeted proximity ligation data, such as focal signal enrichment in contact maps and shorter-range contact frequencies that exhibit the expected (i.e. known in the art) contact decay properties (e.g. Lieberman-Aiden et al, Science, 2009; Cairns et al Genome Biology, 2016). Because it is known in the art, including Applicant's previous inventions listed in the background section, that targeted proximity ligation data can be used to identify genomic rearrangements, this demonstration of high-quality proximity ligation data (via detection of structural features in the data) indicates that data produced from this would also be optimal for the detection of genomic rearrangements.

Row (A) shows the location of genes across the genomic sequences from ~chr17:39,000,000-42,700,000 (according to hg38 human reference genome).

Row (B) shows the sequence coverage, with pronounced peaks in coverage due to the target enrichment of the target genes within this genomic region (~chr17:39,000,000-42,700,000) including CDK12, ERBB2, RARA, SMARCE1, and STAT3.

Rows A and B are also shown vertically, long the y-axis.

Below Rows A and B is a contact map showing the presence of enriched spatial proximity signal emitting from the target genes within this genomic region (~chr17:39,000,000-42,700,000) including CDK12, ERBB2, RARA, SMARCE1, and STAT3. The arrows point to and label these genes along the diagonal of the contact map. Also apparent (and expected) is the distance decay property of the spatial proximity signal, with the signal highest at the target gene, and then decaying (decreasing) in signal intensity (i.e. interaction frequency) as the proximity ligation events between the target gene and neighboring regions get further away in linear proximity. This decaying in signal appears as dissipating “streaks” in signal strength that originate at a target gene, and extend upstream and downstream of the target gene. These “streaks” are most pronounced in this visual from genes CD12, ERBB2, and STAT3, but a common signal feature of all target genes upon closer inspection of individual genes in this genomic region and outside of this genomic region.

### First Detailed Protocol of One Embodiment of the Invention

The below describes a detailed protocol starting with crosslinked cells. Crosslinking in this case was carried out using 2% formaldehyde for 10 min, followed by quenching the crosslinking reaction in 200 mM Glycine.

### A. Samples

B.

TABLE 1

| sample information | |
|---|---|
| Sample | |
| ID | Description |
| 1 | K562_HiC |
| 2 | GM24385_HiC |
| 3 | GM12878_HiC |
| 4 | K562_gDNA |
| 5 | GM24385_gDNA |
| 6 | GM12878_gDNA |
| 7 | K562_sup |
| 8 | GM24385_sup |
| 9 | GM12878_sup |

B. gDNA (this section relates to samples 4-6), the conventional extraction of genomic DNA (not part of the dual library)

i. Lysis

TABLE 2
Lysis Buffer

| | Stock | | | Final | | Master |
|---|---|---|---|---|---|---|
| Reagent | Conc. | Units | uL/rxn | Conc. | Units | Mix |
| Tris-HCl | 1000 | mM | 0.2 | 10 | mM | 0.66 |
| pH 8.0 | | | | | | |
| NaCl | 1000 | mM | 0.2 | 10 | mM | 0.66 |
| IGEPAL | 10 | % | 0.4 | 0.2 | % | 1.32 |
| Protease | 100 | % | 3.33 | 16.67 | % | 11 |
| Inhibitor | | | | | | |
| Cocktail | | | | | | |
| DI Water | | | 15.86667 | | | 52.36 |
| | | Total/rxn | 20 | ul/rxn | | 20 |

0a. Allow crosslinked pellets thaw on ice for at least 15 minutes before adding lysis buffer.

0b. Prepare Lysis Buffer. Place on ice until ready to add in Step 1.

1. Add 20 uL of Lysis Buffer to each sample. **Use pipette to mix around the tissue without aspirating the sample.

2. mix well and incubate samples on ice for at least 15 minutes. —Recommendations: 15 min for cell lines, 20 min for blood, 30 min for tissue, up to 1 hr for difficult samples.

3. Bring volume up to 174 uL with 1× TRIS (add 154 uL)

4. Add 25 uL Proteinase K (800U/mL), 10.5 uL 20% SDS, 20 uL 5M NaCl

5. Incubate in a thermal cycler as follows:
55° C. 30 min Fixed
68° C. 90 min (cells) OR O/N (tissue)
4° C. 10 min OR Hold (10 min minimum to cool sample, up to O/N)

6. If doing SPRI same-day, remove SPRI beads from 4° C. and allow to reach RT (protect from light). (Approx 30 min)

ii. DNA Purification
1. If doing SPRI after O/N hold, remove SPRI beads from 4° C. in AM and allow to reach RT (protect from light). (Approx 30 min)
2. If shearing & size selecting same day, allow Diagenode to cool to 4C during DNA purification.
Add 0.45× SPRI Beads (103.5 uL) and elute in 100 uL 1× Tris

TABLE 3

| SPRI | | |
|---|---|---|
| | DNA | |
| Reagent | Purification | Elution |
| SPRI | 103.5 | — |
| 1X Tris | — | 100 |

3. Volume will exactly fit 0.2 mL PCR tubes if left open during initial addition of SPRI beads; Risk overflow if tubes are closed.
4. SPRI overview Add SPRI beads, mix thoroughly, incubate for 5 min@RT. Magnetize and discard supernatant.
   Wash 2× with freshly prepared 80% EtOH
   Air dry for 2-3 min.
   Add 1× Tris, mix thoroughly, incubate for 5 min @ RT (with caps open to let EtOH continue evap)
   Magnetize and save supernatant.

C. HiC (this section relates to samples 1-3 and 7-9, and is part of the dual library workflow of the invention. This section relates to FIG. 1 Steps b-d).

i. Lysis

0a. Allow crosslinked pellets thaw on ice for at least 15 minutes before adding lysis buffer.

0b. Prepare Lysis Buffer. Place on ice until ready to add in Step 1.

1 Add 20 uL of Lysis Buffer to samples.

TABLE 4
Lysis Buffer

| | Stock | | | Final | | Master |
|---|---|---|---|---|---|---|
| Reagent | Conc. | Units | uL/rxn | Conc. | Units | Mix |
| Tris-HCl | 1000 | mM | 0.17 | 8.33 | mM | 1.65 |
| pH 8.0 | | | | | | |
| NaCl | 1000 | mM | 0.17 | 8.33 | mM | 1.65 |
| IGEPAL | 10 | % | 0.33 | 0.17 | % | 3.3 |
| Protease | 100 | % | 3.33 | 16.67 | % | 11 |
| Inhibitor | | | | | | |
| Cocktail | | | | | | |
| DI Water | | | 16 | | | 158.40 |
| | | Total/rxn | 20 | ul/rxn | 20 | 198 |

2. mix well and incubate samples on ice for at least 15 minutes. —Recommendations: 15 min for cell lines, 20 min for blood, 30 min for frozen (non-ffpe) tissue, up to 1 hr for difficult samples.

2a. Prepare 0.92% SDS and place at RT until ready to add in Step 3.

3. Add 24 UL of 0.92% SDS.

4. Incubate at 62C for 10 min (thermal cycler lid at 85C).

5. Add 20 uL 10.71% TritonX-100.

6. Incubate at 37 C for 15 min (thermal cycler lid at 85C).

6a. Prepare digestion master mix. Place on ice until ready to add in step 7.

TABLE 5
digestion master mix

| | Stock | | | Final |
|---|---|---|---|---|
| Reagent | Conc. | Units | uL/rxn | Conc. |
| NEB3.1 | 10 | x | 7.000 | |
| DpnII | 50,000 | U/ul | 1 | |
| HinfI | 50,000 | U/ul | 4 | |
| | | Total/rxn | 12.000 | ul/rxn |

7. Add 12 uL to each sample of digestion master mix. This step relates to FIG. 1 Step b)

8. Incubate at 37° C. for 30 min for cell lines; 60 min for tissue, then heat inactivate at 62° C. for 20 min (thermal cycler lid at 85C).

9. Prepare fill-in master mix.

TABLE 6
fill-in master mix

| | Stock | | | Final | |
|---|---|---|---|---|---|
| Reagent | Conc. | Units | uL/rxn | Conc. | Units |
| NEB3.1 | 10 | X | 10.000 | 1.000 | X |
| DI water | | | 90.000 | | |
| | | Total | 100.000 | ul | |
| dCTP | 10 | mM | 0.281 | 0.176 | mM |
| dGTP | 10 | mM | 0.281 | 0.176 | mM |
| dTTP | 10 | mM | 0.281 | 0.176 | mM |
| biotin- | 0.4 | mM | 7.013 | 0.175 | mM |
| dATP | | | | | |
| 1X NEB3.1 | 1 | X | 4.144 | 0.259 | X |
| (prepared | | | | | |
| according | | | | | |
| to | | | | | |
| above) | | | | | |
| Klenow | 5 | U/ul | 4.000 | 1.250 | U/ul |
| | | Total/rxn | 16.000 | ul/rxn | |

10. Add 16 uL per sample of fill-in master mix and incubate at RT for 45 min. This step relates to FIG. 1 Step c.

11. Prepare ligation master mix.

TABLE 7
ligation master mix

| | Stock | | | Final | |
|---|---|---|---|---|---|
| Reagent | Conc. | Units | uL/rxn | Conc. | Units |
| 10% | 10 | % | 13.580 | 1.656 | % |
| TritonX- | | | | | |
| 100 | | | | | |
| BSA | 100 | X | 1.650 | 2.012 | X |
| Ligase | 10 | X | 16.500 | 2.012 | X |
| Buffer | | | | | |
| T4 DNA | | | 12.000 | | |
| Ligase | | | | | |
| DI Water | | | 38.270 | | |
| | | Total/rxn | 82.000 | ul/rxn | |

12. Add 16 uL per sample of ligation master mix and incubate at RT (20° C.) for at least 15 min, up to overnight incubation. This step also relates to FIG. 1 Step c.

14. Prepare reverse cross-linking master mix.

TABLE 8
reverse cross-linking master mix

| | Stock | | | Final | | Master |
|---|---|---|---|---|---|---|
| Reagent | Conc. | Units | uL/rxn | Conc. | Units | Mix |
| SDS | 20 | % | 10.500 | 2.561 | % | 46.200 |
| Proteinase K | | | 25.000 | | | 110.000 |
| | | Total/rxn | 35.500 | ul/rxn | | 156.2 |

15. Add 35 uL per sample of reverse crosslinking master mix.

16. Add 20 uL 5M NaCl

17. Incubate in thermal cycler as follows:
55° C. 30 min
68° C. 90 min (cells) OR O/N (blood/tissue)
4° C. 10 min minimum to cool sample, up to O/N

18. If doing SPRI after O/N hold, remove SPRI beads from 4° C. in AM and allow to reach RT (protect from light). (Approx 30 min) 19. If shearing & size selecting same day, allow Diagenode to cool to 4C during DNA purification.

20. Add 0.45× SPRI Beads (103.5 uL) and elute in 100 uL 1× Tris. Volume will exactly fit 0.2 mL PCR tubes if left open during initial addition of SPRI beads; Risk overflow if tubes are closed. 21. SPRI overview:
i. add SPRI beads, mix thoroughly,
ii. incubate for 5 min @ RT. Magnetize and discard supernatant,
iii. wash 2× with freshly prepared 80% EtOH,
iv. air dry for 2-3 min,
v. add 1× Tris, mix thoroughly, incubate for 5 min @ RT (with caps open to let EtOH continue evap), and
vi. magnetize and save supernatant (the supernatant at this step is the purified ligation products of Step d in FIG. 1).

D. Fragmentation (applicable to all samples 1-9, including those of the dual workflow of the claimed invention. This section relates to FIG. 1 Step e):

i. Sonication
1. Shearing Guidelines for DNA Inputs
   Below 500 ng: 1 cycle
   500-1000 ng: 2 cycles
   1000-1500 ng: 3 cycles
   Greater than 1500 ng: 4 cycles
   30 sec ON/90 sec OFF

2. Fragment samples to obtain a peak size of 400 bp. Optionally perform Tapestation DNA size analysis to confirm the DNA size before proceeding.

### E. Size Selection

### Size Selection (200-600 bp Fragment Size)

1. Warm beads to RT prior to use for at least 30 min.

2. Add 1× beads: sample ratio, mix by pipetting and incubate for 5 min at RT.

6. Transfer to a magnet, remove the supernatant

7. Wash with 200 ul 80% EtOH twice.

8. Air dry for 2-3 minutes at RT.

9. Add 50 ul 1× Tris Buffer to elute. Pipet mix to resuspend beads. Incubate at RT for 5 min.

10. Transfer to a magnet and allow solution to become clear. Transfer eluate to a new PCR tube.

11. Quantify the size selected DNA using a Qubit.

F. Library Prep for HiC (fragmented ligated nucleic acids comprising the affinity purification marker) using the SWIFT Accel NGS 2S Plus (now owned by IDT). This section relates to FIG. 1 Step f, g, and h.

### Library Preparation

1 Add 12.5 uL of 10 mg/mL T1 beads to a clean 1.5 mL microfuge tube.

2 Wash the beads by adding 200 uL of 1× Tween Wash Buffer.

3 Separate on magnet and discard the supernatant.

4 Resuspend the beads in 100 uL of 2× BB.

1′ Prepare 100 uL of purified ligated products in 1× Tris.

2′ Add washed T1 beads, resuspended in 100 uL of 2× BB.

3′ Incubate at RT for 15 min.

4′ Magnetize beads until liquid is clear. Remove and SAVE supernatant. This is the separation step where the nucleic acids comprising the affinity purification marker are retained on the magnetized beads and are separated from the nucleic acids not comprising the affinity purification marker which are found in the supernatant.
Set supernatant (containing nucleic acids not comprising the affinity purification marker) aside for eventual DNA purification in Section F below in this protocol.

5 Add 200 uL TWB. Mix by pipetting. Incubate at 55C for 2 min.

6 Magnetize beads until liquid is clear. Remove and discard supernatant.

7 Add 200 uL TWB. Mix by pipetting. Incubate at 55C for 2 min.

8 Magnetize beads until liquid is clear. Remove and discard supernatant.

9 Add 100 uL 1× Tris Buffer. Transfer tube.

10 Magnetize beads until liquid is clear. Remove and discard supernatant. The remaining steps in this section relate to Step h of FIG. 1.

11 Add 40 uL 1× Tris Buffer

12 Add Repair 1 reagents:

| Reagent | uL | MM |
|---|---|---|
| Fragmented, dsDNA | | 40 |
| Low EDTA TE | 13 | 40.95 |
| Buffer W1 | 6 | 18.9 |
| Enzyme W2 | 1 | 3.15 |
| Total | 60 | 63 |

13 mix well and spin down briefly.

14 Incubate in a thermocycler:

| Temp | Time |
|---|---|
| 37 C. | 10 min |

* Set heated lid to OFF

15 Magnetize beads until liquid is clear. Remove and discard supernatant.

16 Add 200 uL TWB. Mix by pipetting. Incubate at 55C for 2 min.

17 Magnetize beads until liquid is clear. Remove and discard supernatant.

18 Add 200 uL TWB. Mix by pipetting. Incubate at 55C for 2 min.

19 Magnetize beads until liquid is clear. Remove and discard supernatant.

20 Add 100 uL 1× Tris Buffer. Transfer tube.

21 Magnetize beads until liquid is clear. Remove and discard supernatant.

22 Resuspend beads in 50 uL each of Repair II master mix

| Reagent | uL | MM |
|---|---|---|
| Low EDTA TE | 30 | 94.5 |
| Buffer G1 | 5 | 15.75 |
| Reagent G2 | 13 | 40.95 |
| Enzyme G3 | 1 | 3.15 |
| Enzyme G4 | 1 | 3.15 |
| Total | 50 | 157.5 |

23 mix well and spin down briefly.

24 Incubate in a thermocycler:

| Temp | Time |
|---|---|
| 20 C. | 20 min |

* Set heated lid to OFF

25 Magnetize beads until liquid is clear. Remove and discard supernatant.

26 Add 200 uL TWB. Mix by pipetting. Incubate at 55C for 2 min.

27 Magnetize beads until liquid is clear. Remove and discard supernatant.

28 Add 200 uL TWB. Mix by pipetting. Incubate at 55C for 2 min.

29 Magnetize beads until liquid is clear. Remove and discard supernatant.

30 Add 100 uL 1× Tris Buffer. Transfer tube.

31 Magnetize beads until liquid is clear. Remove and discard supernatant.

32 Resuspend beads in 25 uL each of Ligation I master mix

| | | |
|---|---|---|
| Reagent | uL | MM |

Reagent Y2 Index X to each sample 5

| | | |
|---|---|---|
| Low EDTA TE | 20 | 63 |
| Buffer Y1 | 3 | 9.45 |
| Enzyme Y3 | 2 | 6.3 |
| Total | 30 | 78.75 |

*Make sure to add both the ligation master mix (25 ul) and unique index (Y2, 5 ul) to each sample.
Be sure to record which Y2 adaptor is used for each sample.

33 mix well and spin down briefly

34 Incubate in a thermocycler:

| Temp | Time |
|---|---|
| 25 C. | 15 min |

* Set heated lid to OFF

35 Magnetize beads until liquid is clear. Remove and discard supernatant.

36 Add 200 uL TWB. Mix by pipetting. Incubate at 55C for 2 min.

37 Magnetize beads until liquid is clear. Remove and discard supernatant.

38 Add 200 uL TWB. Mix by pipetting. Incubate at 55C for 2 min.

39 Magnetize beads until liquid is clear. Remove and discard supernatant.

40 Add 100 uL 1× Tris Buffer. Transfer tube.

41 Magnetize beads until liquid is clear. Remove and discard supernatant.

42 Add 50 uL of Ligation II master mix

| Reagent | uL | MM |
|---|---|---|
| Low EDTA TE | 30 | 94.5 |
| Buffer B1 | 5 | 15.75 |
| Reagent B2-MID | 2 | 6.3 |
| Reagent B3 | 9 | 28.35 |
| Enzyme B4 | 1 | 3.15 |
| Enzyme B5 | 2 | 6.3 |
| Enzyme B6 | 1 | 3.15 |
| Total | 50 | 157.5 |

43 mix well and spin down briefly

44 Incubate in thermocycler:

| Temp | Time |
|---|---|
| 40 C. | 10 min |
| 25 C. | Hold |

* Set heated lid to OFF

45 Magnetize beads until liquid is clear. Remove and discard supernatant.

46 Add 200 uL TWB. Mix by pipetting. Incubate at 55C for 2 min.

47 Magnetize beads until liquid is clear. Remove and discard supernatant.

48 Add 200 uL TWB. Mix by pipetting. Incubate at 55C for 2 min.

49 Magnetize beads until liquid is clear. Remove and discard supernatant.

50 Add 100 uL 1× Tris Buffer. Transfer tube.

51 Magnetize beads until liquid is clear. Remove and discard supernatant.

52 Elute in 22 uL of 1× Tris.

G. Cleanup of supernatant from biotin enrichment for HiC samples; KAPA bead cleanup

1. Warm beads to RT prior to use for at least 30 min.

2. Add 1× beads: sample ratio (200 ul), mix by pipetting and incubate for 5 min at RT.

3. Transfer to a magnet, remove the supernatant.

4. Wash with 800 ul 80% EtOH twice.

5. Air dry for 2-3 minutes at RT.

6. Add 50 ul 1× Tris Buffer to elute. Pipet mix to resuspend beads. Incubate at RT for 10 min.

7. Transfer to a magnet and allow solution to become clear. Transfer eluate to a new PCR tube.

H. Library Preparation: Library Preparation for nucleic acid fragments not comprising the affinity purification marker that were separated during the selective purification step (which was labeled step 4′ of Section E above in this protocol) using SWIFT Accel NGS 2S Plus kit (now owned by IDT). This section relates to step i of FIG. 1.

1 Prepare up to 50 ng of sheared and size-selected ligation products in 1× Tris.

2 Adjust volume to 40 uL with Low EDTA TE

3 Add Repair 1 reagents:

| Reagent | uL | MM |
|---|---|---|
| Fragmented, dsDNA | 40 | |
| Low EDTA TE | 13 | 81.9 |
| Buffer W1 | 6 | 37.8 |
| Enzyme W2 | 1 | 6.3 |
| Total | 60 | 126 |

4 mix well and spin down briefly.

5 Incubate in a thermocycler:

| Temp | Time |
|---|---|
| 37 C. | 10 min |

* Set heated lid to OFF

6 Clean up reaction with 0.7× SPRI (add 42 ul beads). Incubate for 5 min.

7 After EtOH washes, proceed to next step.

8 Resuspend beads in 50 uL each of Repair II master mix.

| Reagent | uL | MM |
|---|---|---|
| Low EDTA TE | 30 | 189 |
| Buffer G1 | 5 | 31.5 |
| Reagent G2 | 13 | 81.9 |
| Enzyme G3 | 1 | 6.3 |
| Enzyme G4 | 1 | 6.3 |
| Total | 50 | 315 |

9 mix well and spin down briefly.

10 Incubate in a thermocycler:

| Temp | Time |
|---|---|
| 20 C. | 20 min |

* Set heated lid to OFF

11 Add 0.55× PEG NaCl (27.5 ul) to reaction. Incubate 5 min for bead cleanup.

12 After EtOH washes, proceed to next step.

13 Resuspend beads in 25 uL each of Ligation I master mix.

| | | |
|---|---|---|
| Reagent | uL | MM |

Reagent Y2 Index X to each sample 5

| | | |
|---|---|---|
| Low EDTA TE | 20 | 126 |
| Buffer Y1 | 3 | 18.9 |
| Enzyme Y3 | 2 | 12.6 |
| Total | 30 | 157.5 |

14 Add 5 ul of unique index (Y2). *Make sure to add both the ligation master mix (25 ul) and unique index (Y2, 5 ul) to each sample. Be sure to record which Y2 adaptor is used for each sample.

15 mix well and spin down briefly

16 Incubate in a thermocycler:

| Temp | Time |
|---|---|
| 25 C. | 15 min |

* Set heated lid to OFF

17 Cleanup ligation reaction. Add 1.2× PEG (36 ul). 50 Incubate 5 min.

18 After EtOH washes, proceed to next step.

19 Add 50 uL of Ligation II master mix

| Reagent | uL | MM |
|---|---|---|
| Low EDTA TE | 30 | 189 |
| Buffer B1 | 5 | 31.5 |
| Reagent B2-MID | 2 | 12.6 |
| Reagent B3 | 9 | 56.7 |
| Enzyme B4 | 1 | 6.3 |
| Enzyme B5 | 2 | 12.6 |
| Enzyme B6 | 1 | 6.3 |
| Total | 50 | 315 |

20 mix well and spin down briefly

21 Incubate in thermocycler:

| Temp | Time |
|---|---|
| 40 C. | 10 min |
| 25 C. | Hold |

* Set heated lid to OFF

22 Cleanup ligation reaction. Add 1.2× PEG (60 ul). Incubate for 5 min.

23 After EtOH washes, proceed to next step.

24 Elute in 22 uL of low EDTA TE buffer 24

I. PCR (this section is applicable to steps j and k in FIG. 1, and was applied to all samples 1-9.)

TABLE 9
KAPA PCR

| Reagent | 1X | MM |
|---|---|---|
| 2X Ready Mix | 25 | 660 |
| 10X Primer Mix | 5 | 132 |
| Sample | 20 | |
| Total | 50/rxn | 30/rxn |

Prepare PCR master mix according to the above table.
Combine 30 uL of master mix with 20 uL of end-repaired and adapter ligated sample (either the output of Section G and Section E according to the above protocol). Perform PCR using cycle conditions recommended by the PCR kit (KAPA) manufacturer.

### PCR Cleanup—0.8× Post PCR SPRI

1 Add beads and incubate 5 min at RT
2 Bind to magnet >=1 min and remove supernatant
3 Add 500 uL 80% EtOH, incubate for >=1 min at RT, remove supernatant
4 Add 500 uL 80% EtOH, incubate for >=1 min at RT, remove supernatant
5 Air Dry for 5 min at RT
6 Add 50 uL 1× Tris Buffer, mix, incubate for >=5 min at RT, transfer tube
7 Repeat steps 1-5 but using a 0.9× SPRI (45 ul)
8 Add 25 uL 1× Tris Buffer, mix, incubate for >=5 min at RT, magnetize and transfer supernatant to new tube

TABLE 10
post-PCR cleanup DNA yields

| | Sample | post-PCR |
|---|---|---|
| ID | Description | ng/uL |
| 1 | K562_HiC | 14.4 |
| 2 | GM24385_HiC | 14.2 |
| 3 | GM12878_HiC | 12.1 |
| 4 | K562_gDNA | 21 |
| 5 | GM24385_gDNA | 21.3 |
| 6 | GM12878_gDNA | 17.2 |
| 7 | K562_sup | 28.8 |
| 8 | GM24385_sup | 20.6 |
| 9 | GM12878_sup | 23.7 |

J. Target enrichment (this section is applicable to steps l and m in FIG. 1. It was not applied in this case but would be applicable to all samples 1-9).

Carry out target enrichment (also known as capture) using target enrichment methods and reagents (probe sets, hybridization buffers, etc.) known in the art, such as using commercial kits from companies such as Agilent Technologies, IDT, Twist Biosciences, etc. This will result in the enrichment of amplified fragments containing the target nucleic acid, and re-amplification of the enriched libraries.

### Second Detailed Protocol of One Embodiment of the Invention

The following detailed protocol is an example of a proximity ligation workflow used in embodiments of methods of the invention for FFPE samples. This protocol relates to the data shown in FIGS. 6-10. Since this section pertains to FFPE samples, the crosslinking has already been completed by the FFPE tissue preparation process (i.e. formalin fixation). The below section covers steps to prepare the FFPE tissue for the proximity ligation workflow (de-waxing and rehydrating the tissue), and also covers steps b-d of FIG. 1.

TABLE 11
sample information

| Sample | | Sample | |
|---|---|---|---|
| ID | Description | type | Pooling |
| 1 | 01SM1 | FFFPE | 2 FFPE 5 um slides |

### A. Dewaxing

1. Collect 1-5 5 μm-10 μm sections and transfer to a clear 1.5 mL tube. **Two pooled 5-10 μm sections work well for a first attempt at a sample. Adjust and pool if yields are lower than expected.

2. Move samples to a fume hood. Add 1 mL of xylene solution to the tube containing the section. **Try to aim just above the tissue and dispense xylene slowly. Ensure the section is doused and in suspension. **Tips are hazardous, eject into appropriately labeled hazardous waste bag for proper disposal

### Choose One:

3a. Standard incubation: Incubate 10 minutes at RT. Invert or vortex every ~3 minutes during incubation.

3b. Overnight xylene: Incubate overnight (>16 hours) at RT with rotation.

4. Centrifuge at max speed (21000×g) for 5 min at RT.

5. Decant xylene from tube. Discard used xylene in appropriate hazardous waste container. **Decant slowly, holding tube at an angle, to avoid loss of sample. **Used xylene should be disposed of in the appropriate waste container. **Use fume hood for decanting.

6. Add 1 mL of 100% EtOH to samples. **Add under fume hood. Tips are nonhazardous.

7. Incubate 10 minutes at RT. Invert or vortex every ~3 minutes during incubation. **Lysis buffer (Step 14) can be prepared during rehydration incubations.

8. Centrifuge at max speed (21000×g) for 5 min at RT.

9. Decant EtOH from tube. **Decant slowly, holding tube at an angle, to avoid loss of sample. **Used ethanol can be disposed of in nonhazardous waste.

10. Add 1 mL DI water to your samples. **If tissue sticks to tube walls easily after rehydration, it's likely from improper wax removal. Take note before proceeding.

11. Incubate 10 minutes at RT. Invert or vortex every ~3 minutes during incubation. **Tissue should appear mostly unraveled by this stage.

12. Centrifuge at max speed (21000×g) for 5 min at RT.

13. Decant DI water from tube. **Tissue is very easy to disturb. Be very cautious during decanting. **Remove as much water as possible but leaving some (<20 uL) is ok to preserve tissue. **May be easier to remove any remaining water with 200 ul pipette.

### B. Lysis

TABLE 12
Lysis Buffer

| | Stock | | | Final | | Master |
|---|---|---|---|---|---|---|
| Reagent | Conc. | Units | uL/rxn | Conc. | Units | Mix |
| Tris-HCl | 1000 | mM | 1.667 | 8.333 | mM | 9.167 |
| pH 8.0 | | | | | | |
| NaCl | 1000 | mM | 1.667 | 8.333 | mM | 9.167 |
| IGEPAL | 10 | % | 3.333 | 0.167 | | 18.333 |
| Protease | 100 | % | 33.333 | 16.667 | % | 183.333 |
| Inhibitor | | | | | | |
| Cocktail | | | | | | |
| DI Water | | | 160.000 | | | 880.000 |
| | | Total/rxn | 200.000 | ul/rxn | | 1100 |

1. Add 200 uL of Lysis Buffer to each sample. **Use pipette to mix around the tissue without aspirating the sample.

2. Incubate samples on ice for 20 minutes. Pipet or vortex briefly every ~5 min to mix. **Cool centrifuge to 4° C. in preparation for the next step.

3. Centrifuge at full speed for 5 min at 4° C.

4. Carefully decant lysis buffer from sample. **Tissue is very easy to disturb. Be very cautious during decanting. Hold samples on ice while decanting many samples. **Remove as much buffer as possible but leaving some (<10 uL) is ok to preserve tissue. **May be easier to remove any remaining supernatant with smaller pipette tips.

5. Add 20 uL 1× Tris pH 7.4. Use volume to transfer FFPE tissue immediately to 8-well PCR strips for Hi-C.

C. HiC (this Sections Covers Step b-d of FIG. 1)

1. Prepare Conditioning Solution

TABLE 13
conditioning solution

| | Stock | | | Final | | Master |
|---|---|---|---|---|---|---|
| Reagent | Conc. | Units | uL/rxn | Conc. | Units | Mix |
| SDS | 20 | % | 1.104 | 0.920 | % | 4.858 |
| DI Water | | | 22.896 | | | 100.742 |
| | | Total/rxn | 24.000 | ul/rxn | | 105.6 |

2. Carefully add 24 ul of conditioning solution to sample without aspirating sample. Pipet mix at ~half of total volume.

3. Incubate at 74° C. for 40 min. Set heated lid to 85° C.

4. Add 20 uL 10.71% TritonX-100. Incubate at 37 C for 15 min. Set heated lid to 85° C.

5. Prepare digestion master mix.

TABLE 14
digestion master mix

| | Stock | | | Final | | Master |
|---|---|---|---|---|---|---|
| Reagent | Conc. | Units | uL/rxn | Conc. | Units | Mix |
| NEB3.1 | 10 | x | 7.000 | | | 30.800 |
| DpnII | 50 | U/ul | 1 | | | 4.400 |
| HinfI | 50 | U/ul | 4 | | | 17.600 |
| | | Total/rxn | 12.000 | ul/rxn | | 52.8 |

6. Carefully add 12 ul of digestion master mix to sample without aspirating sample. Pipet to mix. This step, along with the subsequent step, relate to step b of FIG. 1.

7. Incubate at 37° C. for 1 hour, then heat inactivate at 62° C. for 20 min.

8. Prepare fill-in master mix.

TABLE 15
fill-in master mix

| | Stock | | | Final | | Master |
|---|---|---|---|---|---|---|
| Reagent | Conc. | Units | uL/rxn | Conc. | Units | Mix |
| NEB3.1 | 10 | X | 10.000 | 1.000 | X | 10.000 |
| DI water | | | 90.000 | | | 90.000 |
| | | Total | 100.000 | ul | | 100 |
| dCTP | 10 | mM | 0.281 | 0.176 | mM | 1.236 |
| dGTP | 10 | mM | 0.281 | 0.176 | mM | 1.236 |
| dTTP | 10 | mM | 0.281 | 0.176 | mM | 1.236 |
| biotin- | 0.4 | mM | 7.013 | 0.175 | mM | 30.857 |
| dATP | | | | | | |
| 1X NEB3.1 | 1 | X | 4.144 | 0.259 | X | 18.234 |
| (prepared | | | | | | |
| according | | | | | | |
| to above) | | | | | | |
| Klenow | 5 | U/ul | 4.000 | 1.250 | U/ul | 17.600 |
| | | Total/rxn | 16.000 | ul/rxn | | 70.4 |

9. Carefully add 16 ul of fill-in master mix. Sample should be easier to pipet mix. This step, along with the subsequent step, relate to step c of FIG. 1.

10. Incubate at RT for 45 min.

11. Prepare ligation master mix.

TABLE 16
ligation master mix

| | Stock | | | Final | | Master |
|---|---|---|---|---|---|---|
| Reagent | Conc. | Units | uL/rxn | Conc. | Units | Mix |
| 10% | 10 | % | 13.580 | 1.656 | % | 59.752 |
| TritonX- | | | | | | |
| 100 | | | | | | |
| BSA | 100 | X | 1.650 | 2.012 | X | 7.260 |
| Ligase | 10 | X | 16.500 | 2.012 | X | 72.600 |
| Buffer | | | | | | |
| T4 DNA | | | 12.000 | | | 52.800 |
| Ligase | | | | | | |
| DI Water | | | 38.270 | | | 168.388 |
| | | Total/rxn | 82.000 | ul/rxn | | 360.8 |

12. Carefully add 82 ul of ligation master mix. Sample should be easier to pipet mix. This step, along with the subsequent step, also relate to step c of FIG. 1.

13. Overnight ligation: Incubate in thermocycler at 20° C. overnight (>16 hours). Set heated lid to OFF.

14. Add 16.6 μl 5M NaCl. Incubate at 65° C. overnight (>16 hrs). Set heated lid to 85° C.

15. Prepare reverse cross-linking master mix.

TABLE 17
reverse cross-linking master mix

| | Stock | | | Final | | Master |
|---|---|---|---|---|---|---|
| Reagent | Conc. | Units | uL/rxn | Conc. | Units | Mix |
| SDS | 20 | % | 10.500 | 2.561 | % | 46.200 |
| Proteinase K | | | 25.000 | | | 110.000 |
| | | Total/rxn | 35.500 | ul/rxn | | 156.2 |

16. Carefully add 35.5 ul of reverse cross-linking master mix. Sample should be easier to pipet mix.

17. Incubate at 55° C. overnight (>16 hrs). Set heated lid to 85° C.

18. Add 100 μl (0.45×) SPRI beads to sample. Mix by pipetting and incubate 5 minutes at RT. *Volume should exactly fit in the 0.2 ml strip tubes if you do not close the caps.

19. Transfer to magnet and allow solution to become clear. Discard the supernatant.

20. Wash 2× with 200 ul of 80% EtOH. After second wash, air dry for 2-3 minutes to remove residual EtOH.

21. Add 50 ul 1× Tris buffer. Pipet mix and incubate for 5 min at RT.

22. Transfer to magnet and allow solution to become clear. Transfer eluate to a new 0.2 ml tube.

23. Proceed to preparation for sequencing, including fragmentation, size selection, separating the fragments comprising the affinity purification marker from those not comprising the affinity purification marker, and library preparation (with PCR), and (optionally) target enrichment, according to the methods described above for the non-FFPE cell samples (the first detailed protocol).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

In addition, biological processes are never 100% efficient, so, as one example, fragmenting nucleic acids will likely result in a small portion of nucleic acids that are not fragmented. Likewise, ligation will likely result in a small portion of nucleic acids that remain unligated and so forth. Therefore, when interpreting the claims, it should never be required that all nucleic acids are ligated or amplified, or the like.

Certain implementations of the technology are set forth in the claim(s) that follow(s).

APPENDIX 1

| Cancer Gene | | | | |
|---|---|---|---|---|
| Name | Chromosome | Start | end | strand |
| A1CF | chr10 | 50799409 | 50885675 | − |
| ABI1 | chr10 | 26746593 | 26861087 | − |
| ABI3BP | chr3 | 100749156 | 100993515 | − |
| ABL1 | chr9 | 130713016 | 130887675 | + |
| ABL2 | chr1 | 179099330 | 179229684 | − |
| ABRAXAS1 | chr4 | 83459517 | 83523348 | − |
| ACKR3 | chr2 | 236567787 | 236582354 | + |
| ACSL3 | chr2 | 222860942 | 222944639 | + |
| ACSL6 | chr5 | 131949973 | 132012243 | − |
| ACTB | chr7 | 5526409 | 5563902 | − |
| ACTG1 | chr17 | 81509413 | 81523847 | − |
| ACVR1 | chr2 | 157736251 | 157876330 | − |
| ACVR1B | chr12 | 51951699 | 51997078 | + |
| ACVR2A | chr2 | 147844517 | 147930826 | + |
| ADAMTS20 | chr12 | 43353866 | 43552203 | − |
| ADGRA2 | chr8 | 37784191 | 37844896 | + |
| ADGRB3 | chr6 | 68635282 | 69390571 | + |
| ADGRF5 | chr6 | 46852522 | 46954943 | − |
| ADGRL3 | chr4 | 61200326 | 62078335 | + |
| AFDN | chr6 | 167826911 | 167972023 | + |
| AFF1 | chr4 | 86935002 | 87141039 | + |
| AFF3 | chr2 | 99545419 | 100192428 | − |
| AFF4 | chr5 | 132875395 | 132963634 | − |
| AGO1 | chr1 | 35869808 | 35930532 | + |
| AGO2 | chr8 | 140520156 | 140635633 | − |
| AICDA | chr12 | 8602170 | 8612867 | − |
| AJUBA | chr14 | 22971177 | 22982551 | − |
| AKAP9 | chr7 | 91940840 | 92110673 | + |
| AKT1 | chr14 | 104769349 | 104795751 | − |
| AKT2 | chr19 | 40230317 | 40285536 | − |
| AKT3 | chr1 | 243488233 | 243851079 | − |
| ALB | chr4 | 73397114 | 73421482 | + |
| ALDH2 | chr12 | 111766887 | 111817532 | + |
| ALK | chr2 | 29192774 | 29921586 | − |
| ALOX12B | chr17 | 8072636 | 8087716 | − |
| AMER1 | chrX | 64185117 | 64205708 | − |
| ANK1 | chr8 | 41653220 | 41896762 | − |
| ANKRD11 | chr16 | 89267630 | 89490561 | − |
| ANKRD24 | chr19 | 4183354 | 4224814 | + |
| ANKRD26 | chr10 | 26973793 | 27100494 | − |
| ANKRD30BL | chr2 | 132147591 | 132257969 | − |
| APC | chr5 | 112707498 | 112846239 | + |
| APH1A | chr1 | 150265399 | 150269580 | − |
| APLNR | chr11 | 57233577 | 57237235 | − |
| APOBEC3B | chr22 | 38982347 | 38992804 | + |
| AR | chrX | 67544021 | 67730619 | + |
| ARAF | chrX | 47561205 | 47571908 | + |
| ARFGAP3 | chr22 | 42796502 | 42858106 | − |
| ARFRP1 | chr20 | 63698642 | 63708025 | − |
| ARHGAP26 | chr5 | 142770377 | 143229011 | + |
| ARHGAP35 | chr19 | 46860997 | 47005077 | + |
| ARHGAP5 | chr14 | 32076114 | 32159728 | + |
| ARHGAP6 | chrX | 11117651 | 11665920 | − |
| ARHGEF10 | chr8 | 1823926 | 1958641 | + |
| ARHGEF10L | chr1 | 17539698 | 17697874 | + |
| ARHGEF12 | chr11 | 120336413 | 120489937 | + |
| ARHGEF28 | chr5 | 73626158 | 73941993 | + |
| ARID1A | chr1 | 26693236 | 26782104 | + |
| ARID1B | chr6 | 156776020 | 157210779 | + |
| ARID2 | chr12 | 45729706 | 45908040 | + |
| ARID3A | chr19 | 925781 | 975939 | + |
| ARID3B | chr15 | 74541206 | 74598131 | + |
| ARID3C | chr9 | 34621379 | 34628086 | − |
| ARID4A | chr14 | 58298504 | 58373887 | + |
| ARID4B | chr1 | 235131634 | 235328219 | − |
| ARID5A | chr2 | 96536743 | 96552634 | + |
| ARID5B | chr10 | 61901684 | 62096944 | + |
| ARNT | chr1 | 150809713 | 150876708 | − |
| ASB13 | chr10 | 5638867 | 5666595 | − |
| ASH1L | chr1 | 155335268 | 155563162 | − |
| ASMTL | chrX | 1403139 | 1453762 | − |
| ASPSCR1 | chr17 | 81976807 | 82017406 | + |
| ASXL1 | chr20 | 32358330 | 32439319 | + |
| ASXL2 | chr2 | 25733753 | 25878487 | − |
| ATCAY | chr19 | 3879864 | 3928082 | + |
| ATF1 | chr12 | 50763710 | 50821162 | + |
| ATG5 | chr6 | 106045423 | 106325791 | − |
| ATIC | chr2 | 215311956 | 215349773 | + |
| ATM | chr11 | 108223044 | 108369102 | + |
| ATP1A1 | chr1 | 116372668 | 116410261 | + |
| ATP2B3 | chrX | 153517642 | 153582939 | + |
| ATP5PD | chr17 | 75038863 | 75046985 | − |
| ATP6AP1 | chrX | 154428633 | 154436516 | + |
| ATP6V1B2 | chr8 | 20197381 | 20226819 | + |
| ATR | chr3 | 142449007 | 142578733 | − |
| ATRX | chrX | 77504880 | 77786233 | − |
| ATXN2 | chr12 | 111443485 | 111599676 | − |
| ATXN7 | chr3 | 63863155 | 64003462 | + |
| AURKA | chr20 | 56369389 | 56392337 | − |
| AURKB | chr17 | 8204733 | 8210600 | − |
| AURKC | chr19 | 57230802 | 57235548 | + |
| AUTS2 | chr7 | 69598296 | 70793506 | + |
| AXIN1 | chr16 | 287440 | 352723 | − |
| AXIN2 | chr17 | 65528563 | 65561648 | − |
| AXL | chr19 | 41219223 | 41261766 | + |
| B2M | chr15 | 44711487 | 44718851 | + |
| BABAM1 | chr19 | 17267376 | 17281249 | + |
| BACH2 | chr6 | 89926528 | 90296908 | − |
| BAP1 | chr3 | 52401008 | 52410008 | − |
| BARD1 | chr2 | 214725646 | 214809683 | − |
| BATF3 | chr1 | 212686417 | 212699840 | − |
| BAX | chr19 | 48954815 | 48961798 | + |
| BAZ1A | chr14 | 34752731 | 34875647 | − |
| BBC3 | chr19 | 47220822 | 47232766 | − |
| BCL10 | chr1 | 85265776 | 85276632 | − |
| BCL11A | chr2 | 60450520 | 60554467 | − |
| BCL11B | chr14 | 99169287 | 99272197 | − |
| BCL2 | chr18 | 63123346 | 63320128 | − |
| BCL2A1 | chr15 | 79960892 | 79971196 | − |
| BCL2L1 | chr20 | 31664452 | 31723989 | − |
| BCL2L11 | chr2 | 111119378 | 111168445 | + |
| BCL2L12 | chr19 | 49665142 | 49673916 | + |
| BCL2L2 | chr14 | 23298790 | 23311751 | + |
| BCL3 | chr19 | 44747705 | 44760044 | + |
| BCL6 | chr3 | 187721377 | 187745725 | − |
| BCL7A | chr12 | 122019422 | 122062044 | + |
| BCL9 | chr1 | 147541501 | 147626216 | + |
| BCL9L | chr11 | 118893875 | 118925926 | − |
| BCLAF1 | chr6 | 136256627 | 136289851 | − |
| BCOR | chrX | 40049815 | 40177329 | − |
| BCORL1 | chrX | 129981107 | 130058071 | + |
| BCR | chr22 | 23179704 | 23318037 | + |
| BIRC2 | chr11 | 102347211 | 102378670 | + |
| BIRC3 | chr11 | 102317450 | 102339403 | + |
| BIRC5 | chr17 | 78214186 | 78225636 | + |
| BIRC6 | chr2 | 32357028 | 32618899 | + |
| BLM | chr15 | 90717346 | 90816166 | + |
| BLNK | chr10 | 96189171 | 96271587 | − |
| BLOC1S3 | chr19 | 45178784 | 45216933 | + |
| BMF | chr15 | 40087890 | 40108892 | − |
| BMP5 | chr6 | 55753653 | 55875590 | − |
| BMP7 | chr20 | 57168753 | 57266641 | − |
| BMPR1A | chr10 | 86756601 | 86932825 | + |
| BOD1L1 | chr4 | 13568738 | 13627725 | − |
| BRAF | chr7 | 140719327 | 140924929 | − |
| BRCA1 | chr17 | 43044295 | 43170245 | − |
| BRCA2 | chr13 | 32315086 | 32400268 | + |
| BRD3 | chr9 | 134030305 | 134068535 | − |
| BRD4 | chr19 | 15235519 | 15332545 | − |
| BRINP3 | chr1 | 190097658 | 190478404 | − |
| BRIP1 | chr17 | 61679139 | 61863559 | − |
| BRSK1 | chr19 | 55282072 | 55312562 | + |
| BTG1 | chr12 | 92140278 | 92145846 | − |
| BTG2 | chr1 | 203305491 | 203309602 | + |
| BTK | chrX | 101349447 | 101390796 | − |
| BTLA | chr3 | 112463966 | 112499472 | − |
| BUB1B | chr15 | 40161023 | 40221123 | + |
| C15orf65 | chr15 | 55408495 | 55418798 | + |
| CACNA1D | chr3 | 53328963 | 53813733 | + |
| CACNA1E | chr1 | 181317690 | 181808084 | + |
| CAD | chr2 | 27217369 | 27243943 | + |
| CALR | chr19 | 12938578 | 12944489 | + |
| CAMTA1 | chr1 | 6785454 | 7769706 | + |
| CANT1 | chr17 | 78991716 | 79009867 | − |
| CARD11 | chr7 | 2906142 | 3043867 | − |
| CARM1 | chr19 | 10871553 | 10923075 | + |
| CARS1 | chr11 | 3000922 | 3057613 | − |
| CASP3 | chr4 | 184627696 | 184649509 | − |
| CASP8 | chr2 | 201233443 | 201287711 | + |
| CASP9 | chr1 | 15490832 | 15526534 | − |
| CBFA2T3 | chr16 | 88874858 | 88977207 | − |
| CBFB | chr16 | 67028984 | 67101058 | + |
| CBL | chr11 | 119206298 | 119313926 | + |
| CBLB | chr3 | 105655461 | 105869552 | − |
| CBLC | chr19 | 44777869 | 44800652 | + |
| CCDC50 | chr3 | 191329085 | 191398659 | + |
| CCDC6 | chr10 | 59788747 | 59906556 | − |
| CCN6 | chr6 | 112054075 | 112069686 | + |
| CCNB1IP1 | chr14 | 20311368 | 20333312 | − |
| CCNB3 | chrX | 50202713 | 50351914 | + |
| CCNC | chr6 | 99542387 | 99568825 | − |
| CCND1 | chr11 | 69641156 | 69654474 | + |
| CCND2 | chr12 | 4269771 | 4305353 | + |
| CCND3 | chr6 | 41934934 | 42050357 | − |
| CCNE1 | chr19 | 29811991 | 29824312 | + |
| CCNQ | chrX | 153587925 | 153600045 | − |
| CCR4 | chr3 | 32951644 | 32956349 | + |
| CCR7 | chr17 | 40553769 | 40565472 | − |
| CCT6B | chr17 | 34927859 | 34981078 | − |
| CD209 | chr19 | 7739993 | 7747564 | − |
| CD22 | chr19 | 35319261 | 35347361 | + |
| CD274 | chr9 | 5450503 | 5470566 | + |
| CD276 | chr15 | 73683966 | 73714514 | + |
| CD28 | chr2 | 203706475 | 203738912 | + |
| CD36 | chr7 | 80369575 | 80679277 | + |
| CD44 | chr11 | 35138882 | 35232402 | + |
| CD58 | chr1 | 116514534 | 116571039 | − |
| CD63 | chr12 | 55725323 | 55729707 | − |
| CD70 | chr19 | 6583183 | 6604103 | − |
| CD74 | chr5 | 150400041 | 150412969 | − |
| CD79A | chr19 | 41877279 | 41881372 | + |
| CD79B | chr17 | 63928740 | 63932336 | − |
| CD83 | chr6 | 14117256 | 14136918 | + |
| CDC25A | chr3 | 48157146 | 48188417 | − |
| CDC25C | chr5 | 138285269 | 138338355 | − |
| CDC42 | chr1 | 22025511 | 22101360 | + |
| CDC73 | chr1 | 193121983 | 193254815 | + |
| CDH1 | chr16 | 68737292 | 68835537 | + |
| CDH10 | chr5 | 24487100 | 24644978 | − |
| CDH11 | chr16 | 64943753 | 65126112 | − |
| CDH17 | chr8 | 94127162 | 94217303 | − |
| CDH2 | chr18 | 27932879 | 28177946 | − |
| CDH20 | chr18 | 61333430 | 61555779 | + |
| CDH23 | chr10 | 71396920 | 71815947 | + |
| CDH5 | chr16 | 66366622 | 66404784 | + |
| CDK12 | chr17 | 39461486 | 39564907 | + |
| CDK4 | chr12 | 57747727 | 57756013 | − |
| CDK6 | chr7 | 92604921 | 92836573 | − |
| CDK8 | chr13 | 26254104 | 26405238 | + |
| CDKN1A | chr6 | 36676460 | 36687337 | + |
| CDKN1B | chr12 | 12685498 | 12722369 | + |
| CDKN2A | chr9 | 21967752 | 21995301 | − |
| CDKN2B | chr9 | 22002903 | 22009305 | − |
| CDKN2C | chr1 | 50960745 | 50974634 | + |
| CDX2 | chr13 | 27960918 | 27969315 | − |
| CEBPA | chr19 | 33299934 | 33302534 | − |
| CEBPD | chr8 | 47736913 | 47738164 | − |
| CEBPE | chr14 | 23117306 | 23119255 | − |
| CEBPG | chr19 | 33373685 | 33382686 | + |
| CENPA | chr2 | 26764289 | 26801067 | + |
| CEP43 | chr6 | 166999317 | 167094789 | + |
| CEP89 | chr19 | 32875925 | 32971991 | − |
| CHCHD7 | chr8 | 56211686 | 56218809 | + |
| CHD1 | chr5 | 98853985 | 98929007 | − |
| CHD2 | chr15 | 92900189 | 93027996 | + |
| CHD4 | chr12 | 6570082 | 6614524 | − |
| CHD5 | chr1 | 6101787 | 6180321 | − |
| CHD7 | chr8 | 60678740 | 60868028 | + |
| CHEK1 | chr11 | 125625163 | 125676255 | + |
| CHEK2 | chr22 | 28687743 | 28742422 | − |
| CHIC2 | chr4 | 54009789 | 54064605 | − |
| CHN1 | chr2 | 174798809 | 175005381 | − |
| CHST11 | chr12 | 104455295 | 104762014 | + |
| CIC | chr19 | 42268537 | 42295797 | + |
| CIITA | chr16 | 10866222 | 10943021 | + |
| CILK1 | chr6 | 53001279 | 53061824 | − |
| CKS1B | chr1 | 154974653 | 154979251 | + |
| CLIP1 | chr12 | 122271432 | 122422669 | − |
| CLP1 | chr11 | 57648188 | 57661865 | + |
| CLTC | chr17 | 59619689 | 59696956 | + |
| CLTCL1 | chr22 | 19179473 | 19291719 | − |
| CMPK1 | chr1 | 47333790 | 47378839 | + |
| CMTR2 | chr16 | 71281389 | 71289715 | − |
| CNBD1 | chr8 | 86866415 | 87615219 | + |
| CNBP | chr3 | 129167827 | 129183922 | − |
| CNOT3 | chr19 | 54137749 | 54155681 | + |
| CNTNAP2 | chr7 | 146116002 | 148420998 | + |
| CNTRL | chr9 | 121074753 | 121177610 | + |
| COL1A1 | chr17 | 50184101 | 50201632 | − |
| COL2A1 | chr12 | 47972967 | 48004554 | − |
| COL3A1 | chr2 | 188974373 | 189012746 | + |
| COP1 | chr1 | 175944831 | 176207286 | − |
| COX10 | chr17 | 14069490 | 14231736 | + |
| COX5B | chr2 | 97646062 | 97648383 | + |
| COX6C | chr8 | 99873200 | 99893707 | − |
| CPEB3 | chr10 | 92046692 | 92291078 | − |
| CPS1 | chr2 | 210477682 | 210679107 | + |
| CRBN | chr3 | 3144628 | 3179727 | − |
| CREB1 | chr2 | 207529737 | 207605988 | + |
| CREB3L1 | chr11 | 46277662 | 46321409 | + |
| CREB3L2 | chr7 | 137874979 | 138002086 | − |
| CREBBP | chr16 | 3725054 | 3880713 | − |
| CRKL | chr22 | 20917407 | 20953747 | + |
| CRLF2 | chrX | 1187549 | 1212723 | − |
| CRNKL1 | chr20 | 20034368 | 20056046 | − |
| CRTC1 | chr19 | 18683678 | 18782333 | + |
| CRTC3 | chr15 | 90529923 | 90645345 | + |
| CSDE1 | chr1 | 114716913 | 114758676 | − |
| CSF1 | chr1 | 109910242 | 109930992 | + |
| CSF1R | chr5 | 150053291 | 150113372 | − |
| CSF3R | chr1 | 36466043 | 36483278 | − |
| CSMD3 | chr8 | 112222928 | 113436939 | − |
| CSNK2B | chr6 | 31665227 | 31670343 | + |
| CTCF | chr16 | 67562467 | 67639177 | + |
| CTDNEP1 | chr17 | 7243591 | 7252491 | − |
| CTLA4 | chr2 | 203867771 | 203873965 | + |
| CTNNA1 | chr5 | 138610967 | 138935034 | + |
| CTNNA2 | chr2 | 79185231 | 80648861 | + |
| CTNNB1 | chr3 | 41194741 | 41260096 | + |
| CTNND1 | chr11 | 57753243 | 57819546 | + |
| CTNND2 | chr5 | 10971836 | 11904446 | − |
| CTR9 | chr11 | 10751246 | 10801625 | + |
| CUL3 | chr2 | 224470150 | 224585397 | − |
| CUL4A | chr13 | 113208193 | 113267108 | + |
| CUX1 | chr7 | 101815904 | 102283958 | + |
| CXCR4 | chr2 | 136114349 | 136118149 | − |
| CYB5R2 | chr11 | 7665100 | 7677222 | − |
| CYLD | chr16 | 50742050 | 50801935 | + |
| CYP17A1 | chr10 | 102830531 | 102837472 | − |
| CYP19A1 | chr15 | 51208057 | 51338601 | − |
| CYP2C19 | chr10 | 94762681 | 94855547 | + |
| CYP2C8 | chr10 | 95036772 | 95069497 | − |
| CYP2D6 | chr22 | 42126499 | 42130865 | − |
| CYSLTR2 | chr13 | 48653711 | 48711226 | + |
| DAXX | chr6 | 33318558 | 33323016 | − |
| DCAF12L2 | chrX | 126163499 | 126166289 | − |
| DCC | chr18 | 52340197 | 53535903 | + |
| DCK | chr4 | 70992538 | 71030914 | + |
| DCTN1 | chr2 | 74361154 | 74392087 | − |
| DCUN1D1 | chr3 | 182938074 | 182985953 | − |
| DDB2 | chr11 | 47214465 | 47239217 | + |
| DDIT3 | chr12 | 57516588 | 57521737 | − |
| DDR1 | chr6 | 30876421 | 30900156 | + |
| DDR2 | chr1 | 162631373 | 162787405 | + |
| DDX10 | chr11 | 108665069 | 108940927 | + |
| DDX3X | chrX | 41333348 | 41364472 | + |
| DDX4 | chr5 | 55738017 | 55817157 | + |
| DDX41 | chr5 | 177511577 | 177516961 | − |
| DDX5 | chr17 | 64498254 | 64508199 | − |
| DDX6 | chr11 | 118747763 | 118791164 | − |
| DEK | chr6 | 18223860 | 18264548 | − |
| DENND3 | chr8 | 141117278 | 141195808 | + |
| DGCR8 | chr22 | 20080232 | 20111877 | + |
| DHX15 | chr4 | 24517441 | 24584554 | − |
| DICER1 | chr14 | 95086228 | 95158010 | − |
| DIS3 | chr13 | 72752169 | 72782096 | − |
| DLEU1 | chr13 | 50082169 | 50906856 | + |
| DNAH9 | chr17 | 11598470 | 11969748 | + |
| DNAJB1 | chr19 | 14514769 | 14560391 | − |
| DNM2 | chr19 | 10718055 | 10833488 | + |
| DNMT1 | chr19 | 10133342 | 10231286 | − |
| DNMT3A | chr2 | 25227855 | 25342590 | − |
| DNMT3B | chr20 | 32762385 | 32809356 | + |
| DNTT | chr10 | 96304409 | 96338564 | + |
| DOT1L | chr19 | 2163933 | 2232578 | + |
| DPH3 | chr3 | 16257061 | 16264969 | − |
| DPYD | chr1 | 97077743 | 97995000 | − |
| DROSHA | chr5 | 31400494 | 31532196 | − |
| DST | chr6 | 56457987 | 56954830 | − |
| DTX1 | chr12 | 113056709 | 113098028 | + |
| DUSP2 | chr2 | 96143169 | 96145440 | − |
| DUSP22 | chr6 | 291630 | 351355 | + |
| DUSP4 | chr8 | 29333064 | 29350684 | − |
| DUSP9 | chrX | 153642492 | 153651326 | + |
| DUX4L1 | chr4 | 190084412 | 190085686 | + |
| E2F2 | chr1 | 23506438 | 23531233 | − |
| E2F3 | chr6 | 20401879 | 20493714 | + |
| EBF1 | chr5 | 158695916 | 159099916 | − |
| ECT2L | chr6 | 138795911 | 138904070 | + |
| EED | chr11 | 86244753 | 86278813 | + |
| EGF | chr4 | 109912883 | 110013766 | + |
| EGFL7 | chr9 | 136658856 | 136672678 | + |
| EGFR | chr7 | 55019017 | 55211628 | + |
| EGR1 | chr5 | 138465479 | 138469303 | + |
| EIF1AX | chrX | 20124525 | 20141838 | − |
| EIF3E | chr8 | 108162787 | 108443496 | − |
| EIF4A1 | chr17 | 7572824 | 7579006 | + |
| EIF4A2 | chr3 | 186783205 | 186789897 | + |
| EIF4E | chr4 | 98879276 | 98929133 | − |
| ELF3 | chr1 | 202007945 | 202017183 | + |
| ELF4 | chrX | 130063955 | 130110716 | − |
| ELK4 | chr1 | 205597556 | 205632011 | − |
| ELL | chr19 | 18442663 | 18522116 | − |
| ELN | chr7 | 74027789 | 74069907 | + |
| ELOC | chr8 | 73939169 | 73972287 | − |
| ELP2 | chr18 | 36129444 | 36180557 | + |
| ELP6 | chr3 | 47495640 | 47513712 | − |
| EML4 | chr2 | 42169353 | 42332548 | + |
| EMSY | chr11 | 76444923 | 76553025 | + |
| ENTPD1 | chr10 | 95711779 | 95877266 | + |
| EP300 | chr22 | 41092592 | 41180077 | + |
| EP400 | chr12 | 131949942 | 132080460 | + |
| EPAS1 | chr2 | 46293667 | 46386697 | + |
| EPC1 | chr10 | 32267751 | 32378798 | − |
| EPCAM | chr2 | 47345158 | 47387601 | + |
| EPHA2 | chr1 | 16124337 | 16156069 | − |
| EPHA3 | chr3 | 89107621 | 89482134 | + |
| EPHA5 | chr4 | 65319563 | 65670495 | − |
| EPHA7 | chr6 | 93240020 | 93419687 | − |
| EPHB1 | chr3 | 134795260 | 135260467 | + |
| EPHB4 | chr7 | 100802565 | 100827523 | − |
| EPHB6 | chr7 | 142855061 | 142871094 | + |
| EPOR | chr19 | 11377207 | 11384342 | − |
| EPS15 | chr1 | 51354263 | 51519266 | − |
| ERBB2 | chr17 | 39687914 | 39730426 | + |
| ERBB3 | chr12 | 56076799 | 56103505 | + |
| ERBB4 | chr2 | 211375717 | 212538841 | − |
| ERC1 | chr12 | 990509 | 1495933 | + |
| ERCC1 | chr19 | 45407334 | 45478828 | − |
| ERCC2 | chr19 | 45349837 | 45370918 | − |
| ERCC3 | chr2 | 127257290 | 127294166 | − |
| ERCC4 | chr16 | 13920138 | 13952348 | + |
| ERCC5 | chr13 | 102845831 | 102875995 | + |
| ERF | chr19 | 42247569 | 42255128 | − |
| ERG | chr21 | 38380027 | 38661780 | − |
| ERRFI1 | chr1 | 8004404 | 8026309 | − |
| ESCO2 | chr8 | 27771949 | 27812640 | + |
| ESR1 | chr6 | 151656691 | 152129619 | + |
| ESRRA | chr11 | 64305497 | 64316743 | + |
| ETAA1 | chr2 | 67397322 | 67412089 | + |
| ETNK1 | chr12 | 22625075 | 22690665 | + |
| ETS1 | chr11 | 128458761 | 128587558 | − |
| ETV1 | chr7 | 13891229 | 13991425 | − |
| ETV4 | chr17 | 43527844 | 43579620 | − |
| ETV5 | chr3 | 186046314 | 186110318 | − |
| ETV6 | chr12 | 11649674 | 11895377 | + |
| EVA1C | chr21 | 32412006 | 32515397 | + |
| EWSR1 | chr22 | 29268009 | 29300525 | + |
| EXOC2 | chr6 | 485154 | 693139 | − |
| EXOC7 | chr17 | 76081016 | 76121576 | − |
| EXOSC6 | chr16 | 70246778 | 70251940 | − |
| EXT1 | chr8 | 117794490 | 118111826 | − |
| EXT2 | chr11 | 44095648 | 44251962 | + |
| EZH1 | chr17 | 42700275 | 42745049 | − |
| EZH2 | chr7 | 148807257 | 148884321 | − |
| EZHIP | chrX | 51406948 | 51408843 | + |
| EZR | chr6 | 158765741 | 158819368 | − |
| FAF1 | chr1 | 50437028 | 50960267 | − |
| FAM131B | chr7 | 143353400 | 143362770 | − |
| FAM135B | chr8 | 138130023 | 138497261 | − |
| FAM216A | chr12 | 110468415 | 110490385 | + |
| FAM47C | chrX | 37008366 | 37011664 | + |
| FANCA | chr16 | 89737549 | 89816657 | − |
| FANCC | chr9 | 95099054 | 95426796 | − |
| FANCD2 | chr3 | 10026370 | 10101932 | + |
| FANCE | chr6 | 35452338 | 35467102 | + |
| FANCF | chr11 | 22622533 | 22625823 | − |
| FANCG | chr9 | 35073835 | 35079942 | − |
| FANCL | chr2 | 58159243 | 58241372 | − |
| FAS | chr10 | 88990531 | 89017059 | + |
| FAT1 | chr4 | 186587794 | 186726722 | − |
| FAT3 | chr11 | 92224818 | 92896473 | + |
| FAT4 | chr4 | 125314918 | 125492932 | + |
| FBLN2 | chr3 | 13549125 | 13638422 | + |
| FBXO11 | chr2 | 47789316 | 47906498 | − |
| FBXO31 | chr16 | 87326987 | 87392142 | − |
| FBXW4 | chr10 | 101610664 | 101695295 | − |
| FBXW7 | chr4 | 152320544 | 152536092 | − |
| FCGR2B | chr1 | 161663147 | 161678654 | + |
| FCHO1 | chr19 | 17747718 | 17788568 | + |
| FCRL4 | chr1 | 157573747 | 157598085 | − |
| FEN1 | chr11 | 61792911 | 61797238 | + |
| FES | chr15 | 90883695 | 90895776 | + |
| FEV | chr2 | 218981087 | 218985184 | − |
| FGF1 | chr5 | 142592178 | 142698070 | − |
| FGF10 | chr5 | 44300247 | 44389706 | − |
| FGF12 | chr3 | 192139390 | 192767764 | − |
| FGF14 | chr13 | 101710804 | 102402457 | − |
| FGF19 | chr11 | 69698238 | 69704022 | − |
| FGF23 | chr12 | 4368227 | 4379712 | − |
| FGF3 | chr11 | 69809968 | 69819416 | − |
| FGF4 | chr11 | 69771022 | 69775341 | − |
| FGF6 | chr12 | 4428155 | 4445815 | − |
| FGFR1 | chr8 | 38400215 | 38468834 | − |
| FGFR2 | chr10 | 121478332 | 121598458 | − |
| FGFR3 | chr4 | 1793293 | 1808872 | + |
| FGFR4 | chr5 | 177086905 | 177098144 | + |
| FGR | chr1 | 27612064 | 27635185 | − |
| FH | chr1 | 241497511 | 241519799 | − |
| FHIT | chr3 | 59747277 | 61251459 | − |
| FIP1L1 | chr4 | 53377641 | 53460862 | + |
| FKBP9 | chr7 | 32957404 | 33006930 | + |
| FLCN | chr17 | 17212212 | 17237188 | − |
| FLI1 | chr11 | 128686535 | 128813267 | + |
| FLNA | chrX | 154348524 | 154374634 | − |
| FLT1 | chr13 | 28300346 | 28495145 | − |
| FLT3 | chr13 | 28003274 | 28100592 | − |
| FLT4 | chr5 | 180601506 | 180649624 | − |
| FLYWCH1 | chr16 | 2911931 | 2951208 | + |
| FN1 | chr2 | 215360440 | 215436073 | − |
| FNBP1 | chr9 | 129887187 | 130043194 | − |
| FOS | chr14 | 75278826 | 75282230 | + |
| FOSB | chr19 | 45467995 | 45475179 | + |
| FOXA1 | chr14 | 37589552 | 37596059 | − |
| FOXF1 | chr16 | 86510527 | 86515422 | + |
| FOXL2 | chr3 | 138944224 | 138947137 | − |
| FOXO1 | chr13 | 40555667 | 40666641 | − |
| FOXO3 | chr6 | 108559835 | 108684774 | + |
| FOXO4 | chrX | 71095851 | 71103532 | + |
| FOXP1 | chr3 | 70954708 | 71583989 | − |
| FOXP4 | chr6 | 41546381 | 41602384 | + |
| FOXR1 | chr11 | 118971712 | 118981287 | + |
| FOXR2 | chrX | 55623400 | 55626192 | + |
| FRG2C | chr3 | 75664328 | 75667220 | + |
| FRS2 | chr12 | 69470349 | 69579793 | + |
| FSTL3 | chr19 | 676392 | 683392 | + |
| FUBP1 | chr1 | 77944055 | 77979110 | − |
| FURIN | chr15 | 90868588 | 90883564 | + |
| FUS | chr16 | 31180138 | 31191605 | + |
| FUT8 | chr14 | 65410592 | 65744121 | + |
| FYN | chr6 | 111660332 | 111873452 | − |
| FZR1 | chr19 | 3506311 | 3538334 | + |
| G6PD | chrX | 154531391 | 154547572 | − |
| GAB1 | chr4 | 143336762 | 143474568 | + |
| GAB2 | chr11 | 78215293 | 78418348 | − |
| GABRA6 | chr5 | 161547063 | 161702593 | + |
| GADD45B | chr19 | 2476122 | 2478259 | + |
| GALNT13 | chr2 | 153871922 | 154453979 | + |
| GAS7 | chr17 | 9910606 | 10198606 | − |
| GATA1 | chrX | 48786562 | 48794311 | + |
| GATA2 | chr3 | 128479427 | 128493201 | − |
| GATA3 | chr10 | 8045378 | 8075198 | + |
| GATA4 | chr8 | 11676959 | 11760002 | + |
| GATA6 | chr18 | 22169589 | 22202528 | + |
| GDNF | chr5 | 37812677 | 37840041 | − |
| GID4 | chr17 | 18039408 | 18068405 | + |
| GIPR | chr19 | 45668221 | 45683722 | + |
| GLI1 | chr12 | 57459785 | 57472268 | + |
| GLIS2 | chr16 | 4314761 | 4339597 | + |
| GMPS | chr3 | 155870650 | 155944020 | + |
| GNA11 | chr19 | 3094362 | 3123999 | + |
| GNA12 | chr7 | 2728105 | 2844308 | − |
| GNA13 | chr17 | 65009289 | 65056740 | − |
| GNAI3 | chr1 | 109548615 | 109600195 | + |
| GNAQ | chr9 | 77716097 | 78031811 | − |
| GNAS | chr20 | 58839718 | 58911192 | + |
| GNB1 | chr1 | 1785285 | 1891117 | − |
| GOLGA5 | chr14 | 92794305 | 92839947 | + |
| GOPC | chr6 | 117560269 | 117602542 | − |
| GPC3 | chrX | 133535745 | 133985594 | − |
| GPC5 | chr13 | 91398621 | 92873682 | + |
| GPHN | chr14 | 66507407 | 67181803 | + |
| GPS2 | chr17 | 7311324 | 7315564 | − |
| GRB7 | chr17 | 39737927 | 39747291 | + |
| GREM1 | chr15 | 32718004 | 32745106 | + |
| GRIN2A | chr16 | 9753404 | 10182928 | − |
| GRM3 | chr7 | 86643909 | 86864879 | + |
| GRM8 | chr7 | 126438598 | 127253093 | − |
| GSE1 | chr16 | 85169525 | 85676204 | + |
| GSK3B | chr3 | 119821321 | 120094994 | − |
| GTF21 | chr7 | 74650231 | 74760692 | + |
| GTSE1 | chr22 | 46296870 | 46330810 | + |
| GUCY1A2 | chr11 | 106674019 | 107018476 | − |
| H1-2 | chr6 | 26055740 | 26056470 | − |
| H1-3 | chr6 | 26234212 | 26234987 | − |
| H1-4 | chr6 | 26156329 | 26157115 | + |
| H1-5 | chr6 | 27866792 | 27867588 | − |
| H2AC11 | chr6 | 27133042 | 27135291 | + |
| H2AC16 | chr6 | 27865317 | 27865798 | + |
| H2AC17 | chr6 | 27892699 | 27893185 | − |
| H2AC6 | chr6 | 26124145 | 26139116 | + |
| H2BC11 | chr6 | 27125897 | 27132795 | − |
| H2BC12 | chr6 | 27146361 | 27146855 | − |
| H2BC17 | chr6 | 27893425 | 27893891 | + |
| H2BC4 | chr6 | 26114873 | 26123926 | − |
| H2BC5 | chr6 | 26158146 | 26171349 | + |
| H2BC8 | chr6 | 26215159 | 26216692 | − |
| H3-3A | chr1 | 226061851 | 226072019 | + |
| H3-3B | chr17 | 75776434 | 75785893 | − |
| H3-4 | chr1 | 228424845 | 228425360 | − |
| H3-5 | chr12 | 31791185 | 31792298 | − |
| H3C1 | chr6 | 26020451 | 26020958 | + |
| H3C10 | chr6 | 27810064 | 27811300 | + |
| H3C11 | chr6 | 27871845 | 27872346 | − |
| H3C12 | chr6 | 27890315 | 27893106 | − |
| H3C13 | chr1 | 149813225 | 149813693 | − |
| H3C14 | chr1 | 149839538 | 149841193 | − |
| H3C2 | chr6 | 26031589 | 26032099 | − |
| H3C3 | chr6 | 26045384 | 26045869 | + |
| H3C4 | chr6 | 26196784 | 26197286 | − |
| H3C6 | chr6 | 26224199 | 26227473 | + |
| H3C7 | chr6 | 26250142 | 26250635 | − |
| H3C8 | chr6 | 26269405 | 26271815 | − |
| H4C9 | chr6 | 27138588 | 27139881 | + |
| HAUS3 | chr4 | 2078998 | 2242276 | − |
| HCAR1 | chr12 | 122726076 | 122730844 | − |
| HDAC1 | chr1 | 32292083 | 32333635 | + |
| HDAC4 | chr2 | 239048168 | 239401654 | − |
| HDAC7 | chr12 | 47782722 | 47833132 | − |
| HERPUD1 | chr16 | 56932142 | 56944864 | + |
| HEY1 | chr8 | 79762371 | 79767857 | − |
| HGF | chr7 | 81699010 | 81770438 | − |
| HIF1A | chr14 | 61695513 | 61748259 | + |
| HIF3A | chr19 | 46297042 | 46343433 | + |
| HIP1 | chr7 | 75533298 | 75738962 | − |
| HIRA | chr22 | 19330698 | 19447450 | − |
| HLA-A | chr6 | 29941260 | 29945884 | + |
| HLA-B | chr6 | 31353872 | 31357188 | − |
| HLA-C | chr6 | 31268749 | 31272130 | − |
| HLF | chr17 | 55264960 | 55325187 | + |
| HMGA1 | chr6 | 34236873 | 34246231 | + |
| HMGA2 | chr12 | 65824460 | 65966291 | + |
| HMGCL | chr1 | 23801885 | 23838620 | − |
| HMGN2P46 | chr15 | 45511136 | 45586290 | + |
| HNF1A | chr12 | 120978543 | 121002512 | + |
| HNRNPA2B1 | chr7 | 26171151 | 26201529 | − |
| HNRNPK | chr9 | 83968083 | 83980616 | − |
| HOOK3 | chr8 | 42896946 | 43030535 | + |
| HOXA10 | chr7 | 27170592 | 27180261 | − |
| HOXA11 | chr7 | 27181157 | 27185232 | − |
| HOXA13 | chr7 | 27193503 | 27200091 | − |
| HOXA3 | chr7 | 27106184 | 27152583 | − |
| HOXA9 | chr7 | 27162438 | 27175180 | − |
| HOXB13 | chr17 | 48724763 | 48728750 | − |
| HOXC11 | chr12 | 53973126 | 53977643 | + |
| HOXC13 | chr12 | 53938831 | 53946544 | + |
| HOXD11 | chr2 | 176104216 | 176109754 | + |
| HOXD13 | chr2 | 176092721 | 176095944 | + |
| HRAS | chr11 | 532242 | 537321 | − |
| HSD3B1 | chr1 | 119507198 | 119515054 | + |
| HSP90AA1 | chr14 | 102080742 | 102139699 | − |
| HSP90AB1 | chr6 | 44246166 | 44253888 | + |
| ICOSLG | chr21 | 44217014 | 44240966 | − |
| ID3 | chr1 | 23557926 | 23559501 | − |
| ID4 | chr6 | 19837370 | 19842197 | + |
| IDH1 | chr2 | 208236229 | 208266074 | − |
| IDH2 | chr15 | 90083045 | 90102477 | − |
| IFNGR1 | chr6 | 137197485 | 137219449 | − |
| IGF1 | chr12 | 102395874 | 102481744 | − |
| IGF1R | chr15 | 98648539 | 98964530 | + |
| IGF2 | chr11 | 2129112 | 2141238 | − |
| IGF2BP2 | chr3 | 185643130 | 185825042 | − |
| IGF2R | chr6 | 159969082 | 160113507 | + |
| IGHA1 | chr14 | 105703995 | 105708665 | − |
| IGHA2 | chr14 | 105583731 | 105588395 | − |
| IGHG1 | chr14 | 105736343 | 105743071 | − |
| IGHG2 | chr14 | 105639559 | 105644790 | − |
| IGHG3 | chr14 | 105764503 | 105771405 | − |
| IGHG4 | chr14 | 105620506 | 105626066 | − |
| IGHJ1 | chr14 | 105865407 | 105865458 | − |
| IGHJ2 | chr14 | 105865199 | 105865250 | − |
| IGHJ3 | chr14 | 105864587 | 105864635 | − |
| IGHJ4 | chr14 | 105864215 | 105864260 | − |
| IGHJ5 | chr14 | 105863814 | 105863862 | − |
| IGHJ6 | chr14 | 105863198 | 105863258 | − |
| IGHM | chr14 | 105851705 | 105856218 | − |
| IKBKB | chr8 | 42271302 | 42332460 | + |
| IKBKE | chr1 | 206470476 | 206496889 | + |
| IKZF1 | chr7 | 50304068 | 50405101 | + |
| IKZF2 | chr2 | 212999691 | 213152427 | − |
| IKZF3 | chr17 | 39757718 | 39864312 | − |
| IL10 | chr1 | 206767602 | 206774541 | − |
| IL16 | chr15 | 81159575 | 81314058 | + |
| IL2 | chr4 | 122451470 | 122456725 | − |
| IL21R | chr16 | 27402174 | 27452042 | + |
| IL3 | chr5 | 132060655 | 132063204 | + |
| IL6ST | chr5 | 55935095 | 55995022 | − |
| IL7R | chr5 | 35852695 | 35879603 | + |
| ING4 | chr12 | 6650301 | 6663142 | − |
| INHA | chr2 | 219569162 | 219575711 | + |
| INHBA | chr7 | 41667168 | 41705834 | − |
| INPP4A | chr2 | 98444854 | 98594392 | + |
| INPP4B | chr4 | 142023160 | 142847432 | − |
| INPP5D | chr2 | 233059967 | 233207903 | + |
| INPPL1 | chr11 | 72223701 | 72239147 | + |
| INSR | chr19 | 7112255 | 7294414 | − |
| IRAG2 | chr12 | 25004342 | 25108334 | + |
| IRF1 | chr5 | 132440440 | 132508719 | − |
| IRF2 | chr4 | 184387729 | 184474550 | − |
| IRF4 | chr6 | 391752 | 411443 | + |
| IRF8 | chr16 | 85899162 | 85922606 | + |
| IRS1 | chr2 | 226731312 | 226799820 | − |
| IRS2 | chr13 | 109752695 | 109786583 | − |
| IRS4 | chrX | 108719946 | 108736563 | − |
| ISX | chr22 | 35066136 | 35087387 | + |
| ITGA10 | chr1 | 145891208 | 145910111 | − |
| ITGA9 | chr3 | 37452115 | 37823507 | + |
| ITGAV | chr2 | 186590056 | 186680901 | + |
| ITGB2 | chr21 | 44885953 | 44931989 | − |
| ITGB3 | chr17 | 47253827 | 47313743 | + |
| ITK | chr5 | 157142933 | 157255185 | + |
| ITPKB | chr1 | 226631690 | 226739323 | − |
| JAK1 | chr1 | 64833229 | 65067754 | − |
| JAK2 | chr9 | 4984390 | 5129948 | + |
| JAK3 | chr19 | 17824780 | 17848071 | − |
| JARID2 | chr6 | 15246069 | 15522042 | + |
| JAZF1 | chr7 | 27830573 | 28180795 | − |
| JUN | chr1 | 58776845 | 58784048 | − |
| KAT6A | chr8 | 41929479 | 42051994 | − |
| KAT6B | chr10 | 74824927 | 75032624 | + |
| KAT7 | chr17 | 49788648 | 49835026 | + |
| KBTBD4 | chr11 | 47572197 | 47578976 | − |
| KCNJ5 | chr11 | 128891356 | 128921163 | − |
| KDM2B | chr12 | 121429096 | 121581023 | − |
| KDM4C | chr9 | 6720863 | 7175648 | + |
| KDM5A | chr12 | 280057 | 389320 | − |
| KDM5C | chrX | 53191321 | 53225422 | − |
| KDM6A | chrX | 44873188 | 45112779 | + |
| KDR | chr4 | 55078481 | 55125595 | − |
| KDSR | chr18 | 63327726 | 63367228 | − |
| KEAP1 | chr19 | 10486125 | 10503558 | − |
| KEL | chr7 | 142941114 | 142962363 | − |
| KIAA1549 | chr7 | 138831381 | 138981389 | − |
| KIF5B | chr10 | 32009015 | 32056425 | − |
| KIT | chr4 | 54657918 | 54740715 | + |
| KLF15 | chr3 | 126342635 | 126357408 | − |
| KLF2 | chr19 | 16324826 | 16328685 | + |
| KLF3 | chr4 | 38664197 | 38701517 | + |
| KLF4 | chr9 | 107484852 | 107490482 | − |
| KLF5 | chr13 | 73054976 | 73077541 | + |
| KLF6 | chr10 | 3775996 | 3785281 | − |
| KLHL6 | chr3 | 183487551 | 183555706 | − |
| KLK2 | chr19 | 50861568 | 50880567 | + |
| KMT2A | chr11 | 118436490 | 118526832 | + |
| KMT2B | chr19 | 35717973 | 35738878 | + |
| KMT2C | chr7 | 152134922 | 152436644 | − |
| KMT2D | chr12 | 49018978 | 49060794 | − |
| KMT5A | chr12 | 123383773 | 123409353 | + |
| KNL1 | chr15 | 40594020 | 40664342 | + |
| KNSTRN | chr15 | 40382721 | 40394246 | + |
| KRAS | chr12 | 25205246 | 25250936 | − |
| KSR2 | chr12 | 117453012 | 117968990 | − |
| KTN1 | chr14 | 55559072 | 55701526 | + |
| LAMP1 | chr13 | 113297239 | 113323672 | + |
| LARP4B | chr10 | 806914 | 931705 | − |
| LASP1 | chr17 | 38869859 | 38921770 | + |
| LATS1 | chr6 | 149658153 | 149718105 | − |
| LATS2 | chr13 | 20973036 | 21061586 | − |
| LCK | chr1 | 32251239 | 32286165 | + |
| LCP1 | chr13 | 46125920 | 46211871 | − |
| LEF1 | chr4 | 108047545 | 108168956 | − |
| LEPROTL1 | chr8 | 30095408 | 30177208 | + |
| LHFPL6 | chr13 | 39209116 | 39603528 | − |
| LIFR | chr5 | 38474668 | 38608354 | − |
| LIMD1 | chr3 | 45555394 | 45686341 | + |
| LMNA | chr1 | 156082573 | 156140081 | + |
| LMO1 | chr11 | 8224309 | 8268716 | − |
| LMO2 | chr11 | 33858576 | 33892076 | − |
| LPP | chr3 | 188153284 | 188890671 | + |
| LRIG3 | chr12 | 58872149 | 58920504 | − |
| LRP1B | chr2 | 140231423 | 142131016 | − |
| LRP5 | chr11 | 68312591 | 68449275 | + |
| LRP6 | chr12 | 12116025 | 12267044 | − |
| LRRK2 | chr12 | 40196744 | 40369285 | + |
| LSM14A | chr19 | 34172504 | 34229288 | + |
| LTB | chr6 | 31580525 | 31582522 | − |
| LTF | chr3 | 46435645 | 46485234 | − |
| LTK | chr15 | 41503637 | 41513887 | − |
| LUC7L2 | chr7 | 139340359 | 139423457 | + |
| LYL1 | chr19 | 13099033 | 13103161 | − |
| LYN | chr8 | 55879835 | 56014169 | + |
| LZTR1 | chr22 | 20982269 | 20999032 | + |
| LZTS1 | chr8 | 20246165 | 20303963 | − |
| MACC1 | chr7 | 20134655 | 20217404 | − |
| MAD2L2 | chr1 | 11674480 | 11691650 | − |
| MAF | chr16 | 79585843 | 79600737 | − |
| MAFB | chr20 | 40685848 | 40689236 | − |
| MAGEA1 | chrX | 153179284 | 153183880 | + |
| MAGED1 | chrX | 51803007 | 51902354 | + |
| MAGI1 | chr3 | 65353525 | 66038834 | − |
| MAL | chr2 | 95025677 | 95053992 | + |
| MALAT1 | chr11 | 65497688 | 65506516 | + |
| MALT1 | chr18 | 58671465 | 58754477 | + |
| MAML2 | chr11 | 95976598 | 96343195 | − |
| MAML3 | chr4 | 139716753 | 140154184 | − |
| MAP2K1 | chr15 | 66386837 | 66491544 | + |
| MAP2K2 | chr19 | 4090321 | 4124122 | − |
| MAP2K4 | chr17 | 12020829 | 12143830 | + |
| MAP3K1 | chr5 | 56815549 | 56896152 | + |
| MAP3K13 | chr3 | 185282941 | 185489094 | + |
| MAP3K14 | chr17 | 45263119 | 45317029 | − |
| MAP3K6 | chr1 | 27354067 | 27366961 | − |
| MAP3K7 | chr6 | 90513573 | 90587072 | − |
| MAPK1 | chr22 | 21759657 | 21867680 | − |
| MAPK3 | chr16 | 30114105 | 30123506 | − |
| MAPK8 | chr10 | 48306639 | 48439360 | + |
| MAPKAP1 | chr9 | 125437393 | 125707234 | − |
| MARK1 | chr1 | 220528136 | 220664461 | + |
| MARK4 | chr19 | 45079288 | 45305284 | + |
| MAST1 | chr19 | 12833951 | 12874952 | + |
| MAST2 | chr1 | 45786987 | 46036122 | + |
| MAX | chr14 | 65006174 | 65102695 | − |
| MB21D2 | chr3 | 192796815 | 192917856 | − |
| MBD1 | chr18 | 50266882 | 50281774 | − |
| MBD6 | chr12 | 57520710 | 57530148 | + |
| MBTD1 | chr17 | 51177425 | 51260163 | − |
| MCL1 | chr1 | 150560895 | 150579738 | − |
| MDC1 | chr6 | 30699807 | 30717447 | − |
| MDM2 | chr12 | 68808177 | 68845544 | + |
| MDM4 | chr1 | 204516379 | 204558120 | + |
| MDS2 | chr1 | 23581495 | 23640568 | + |
| MEAF6 | chr1 | 37489993 | 37514766 | − |
| MECOM | chr3 | 169083499 | 169663775 | − |
| MED12 | chrX | 71118556 | 71142454 | + |
| MEF2B | chr19 | 19145567 | 19192131 | − |
| MEF2C | chr5 | 88717117 | 88904257 | − |
| MEF2D | chr1 | 156463727 | 156500779 | − |
| MEN1 | chr11 | 64803510 | 64811294 | − |
| MERTK | chr2 | 111898607 | 112029561 | + |
| MET | chr7 | 116672196 | 116798377 | + |
| METAP1D | chr2 | 171999943 | 172082430 | + |
| MGA | chr15 | 41621134 | 41773081 | + |
| MGAM | chr7 | 141907813 | 142106747 | + |
| MGMT | chr10 | 129467190 | 129770983 | + |
| MIB1 | chr18 | 21704957 | 21870953 | + |
| MIDEAS | chr14 | 73715122 | 73790285 | − |
| MIR663AHG | chr20 | 26167817 | 26251546 | − |
| MITF | chr3 | 69739464 | 69968336 | + |
| MKI67 | chr10 | 128096659 | 128126423 | − |
| MKNK1 | chr1 | 46557407 | 46616843 | − |
| MLF1 | chr3 | 158571163 | 158607252 | + |
| MLH1 | chr3 | 36993350 | 37050846 | + |
| MLH3 | chr14 | 75013769 | 75051532 | − |
| MLLT1 | chr19 | 6210381 | 6279975 | − |
| MLLT10 | chr10 | 21524646 | 21743630 | + |
| MLLT11 | chr1 | 151060397 | 151069544 | + |
| MLLT3 | chr9 | 20341669 | 20622499 | − |
| MLLT6 | chr17 | 38705273 | 38729795 | + |
| MME | chr3 | 155024124 | 155183704 | + |
| MMP2 | chr16 | 55389700 | 55506691 | + |
| MN1 | chr22 | 27748277 | 27801756 | − |
| MNX1 | chr7 | 156994051 | 157010663 | − |
| MOB3B | chr9 | 27325209 | 27529814 | − |
| MPEG1 | chr11 | 59208510 | 59212927 | − |
| MPL | chr1 | 43337818 | 43354466 | + |
| MPV17L2 | chr19 | 18193218 | 18196948 | + |
| MRE11 | chr11 | 94415570 | 94493885 | − |
| MRTFA | chr22 | 40410281 | 40636719 | − |
| MRTFB | chr16 | 14071319 | 14266773 | + |
| MSH2 | chr2 | 47403067 | 47663146 | + |
| MSH3 | chr5 | 80654652 | 80876815 | + |
| MSH6 | chr2 | 47695530 | 47810063 | + |
| MSI1 | chr12 | 120341330 | 120369164 | − |
| MSI2 | chr17 | 57255851 | 57684689 | + |
| MSMB | chr10 | 46033307 | 46048180 | − |
| MSN | chrX | 65588377 | 65741931 | + |
| MST1 | chr3 | 49683947 | 49689501 | − |
| MST1R | chr3 | 49887002 | 49903873 | − |
| MTAP | chr9 | 21802636 | 21937651 | + |
| MTCP1 | chrX | 155064034 | 155147937 | − |
| MTOR | chr1 | 11106535 | 11262551 | − |
| MTR | chr1 | 236795260 | 236921278 | + |
| MTRR | chr5 | 7851186 | 7906025 | + |
| MUC1 | chr1 | 155185824 | 155192916 | − |
| MUC16 | chr19 | 8848844 | 8981342 | − |
| MUC4 | chr3 | 195746765 | 195811973 | − |
| MUSK | chr9 | 110668779 | 110806558 | + |
| MUTYH | chr1 | 45329163 | 45340893 | − |
| MYB | chr6 | 135181308 | 135219173 | + |
| MYBL1 | chr8 | 66562175 | 66614247 | − |
| MYC | chr8 | 127735434 | 127742951 | + |
| MYCL | chr1 | 39895426 | 39902256 | − |
| MYCN | chr2 | 15940550 | 15947007 | + |
| MYD88 | chr3 | 38138478 | 38143022 | + |
| MYH11 | chr16 | 15703135 | 15857028 | − |
| MYH9 | chr22 | 36281280 | 36387967 | − |
| MYO18A | chr17 | 29071122 | 29180398 | − |
| MYO19 | chr17 | 36495636 | 36543435 | − |
| MYO5A | chr15 | 52307283 | 52529050 | − |
| MYOD1 | chr11 | 17719571 | 17722136 | + |
| N4BP2 | chr4 | 40056850 | 40158252 | + |
| NAB2 | chr12 | 57089043 | 57095476 | + |
| NACA | chr12 | 56712305 | 56731628 | − |
| NADK | chr1 | 1751232 | 1780457 | − |
| NBEA | chr13 | 34942270 | 35673022 | + |
| NBEAP1 | chr15 | 20657638 | 20688408 | − |
| NBN | chr8 | 89933331 | 90003228 | − |
| NCKIPSD | chr3 | 48673844 | 48686364 | − |
| NCOA1 | chr2 | 24491254 | 24770702 | + |
| NCOA2 | chr8 | 70109782 | 70403808 | − |
| NCOA3 | chr20 | 47501887 | 47656877 | + |
| NCOA4 | chr10 | 46005088 | 46030623 | − |
| NCOR1 | chr17 | 16029157 | 16218185 | − |
| NCOR2 | chr12 | 124324415 | 124567589 | − |
| NCSTN | chr1 | 160343316 | 160358952 | + |
| NDRG1 | chr8 | 133237175 | 133302022 | − |
| NDUFS7 | chr19 | 1383527 | 1395589 | + |
| NEGR1 | chr1 | 71395943 | 72282539 | − |
| NEK6 | chr9 | 124257606 | 124353307 | + |
| NF1 | chr17 | 31094927 | 31382116 | + |
| NF2 | chr22 | 29603556 | 29698598 | + |
| NFATC2 | chr20 | 51386957 | 51562831 | − |
| NFE2 | chr12 | 54292111 | 54301015 | − |
| NFE2L2 | chr2 | 177227595 | 177392697 | − |
| NFIB | chr9 | 14081843 | 14398983 | − |
| NFKB1 | chr4 | 102501331 | 102617302 | + |
| NFKB2 | chr10 | 102394110 | 102402524 | + |
| NFKBIA | chr14 | 35401513 | 35404749 | − |
| NFKBIE | chr6 | 44258166 | 44265788 | − |
| NIN | chr14 | 50719763 | 50831162 | − |
| NKX2-1 | chr14 | 36516392 | 36521149 | − |
| NKX3-1 | chr8 | 23678697 | 23682938 | − |
| NLRP1 | chr17 | 5499427 | 5619424 | − |
| NME1 | chr17 | 51153559 | 51162428 | + |
| NOD1 | chr7 | 30424527 | 30478784 | − |
| NONO | chrX | 71254814 | 71301522 | + |
| NOTCH1 | chr9 | 136494433 | 136546048 | − |
| NOTCH2 | chr1 | 119911553 | 120100779 | − |
| NOTCH3 | chr19 | 15159038 | 15200995 | − |
| NOTCH4 | chr6 | 32194843 | 32224067 | − |
| NPM1 | chr5 | 171387116 | 171411810 | + |
| NR4A3 | chr9 | 99821855 | 99866891 | + |
| NRAS | chr1 | 114704469 | 114716771 | − |
| NRG1 | chr8 | 31639222 | 32855666 | + |
| NSD1 | chr5 | 177133025 | 177300213 | + |
| NSD2 | chr4 | 1871393 | 1982207 | + |
| NSD3 | chr8 | 38269704 | 38382272 | − |
| NT5C2 | chr10 | 103087185 | 103277605 | − |
| NTHL1 | chr16 | 2039815 | 2047866 | − |
| NTRK1 | chr1 | 156815640 | 156881850 | + |
| NTRK2 | chr9 | 84668551 | 85027054 | + |
| NTRK3 | chr15 | 87859751 | 88256768 | − |
| NUF2 | chr1 | 163266576 | 163355764 | + |
| NUMA1 | chr11 | 72002864 | 72080693 | − |
| NUMBL | chr19 | 40665905 | 40690972 | − |
| NUP214 | chr9 | 131125586 | 131234663 | + |
| NUP93 | chr16 | 56730118 | 56850286 | + |
| NUP98 | chr11 | 3671083 | 3797792 | − |
| NUTM1 | chr15 | 34343315 | 34357737 | + |
| NUTM2A | chr10 | 87225448 | 87236908 | + |
| NUTM2B | chr10 | 79703227 | 79714681 | + |
| NUTM2D | chr10 | 87357720 | 87370695 | + |
| NXF1 | chr11 | 62792123 | 62806302 | − |
| OGA | chr10 | 101784443 | 101818465 | − |
| OLIG2 | chr21 | 33025935 | 33029196 | + |
| OMD | chr9 | 92412380 | 92424471 | − |
| P2RY8 | chrX | 1462581 | 1537185 | − |
| PABPC1 | chr8 | 100685816 | 100722809 | − |
| PAFAH1B2 | chr11 | 117144284 | 117176894 | + |
| PAG1 | chr8 | 80967810 | 81112068 | − |
| PAICS | chr4 | 56435741 | 56464578 | + |
| PAK1 | chr11 | 77322017 | 77474635 | − |
| PAK3 | chrX | 110944285 | 111227361 | + |
| PAK5 | chr20 | 9537370 | 9839076 | − |
| PALB2 | chr16 | 23603160 | 23641310 | − |
| PARP1 | chr1 | 226360210 | 226408154 | − |
| PARP2 | chr14 | 20343615 | 20357904 | + |
| PARP3 | chr3 | 51942345 | 51948867 | + |
| PASK | chr2 | 241106099 | 241150264 | − |
| PATZ1 | chr22 | 31325804 | 31346346 | − |
| PAX3 | chr2 | 222199887 | 222298998 | − |
| PAX5 | chr9 | 36833269 | 37034268 | − |
| PAX7 | chr1 | 18630846 | 18748866 | + |
| PAX8 | chr2 | 113215997 | 113278921 | − |
| PBRM1 | chr3 | 52545352 | 52685917 | − |
| PBX1 | chr1 | 164555584 | 164899296 | + |
| PC | chr11 | 66848417 | 66958386 | − |
| PCBP1 | chr2 | 70087477 | 70089203 | + |
| PCDHAC2 | chr5 | 140966470 | 141012347 | + |
| PCLAF | chr15 | 64364304 | 64387687 | − |
| PCLO | chr7 | 82754012 | 83162930 | − |
| PCM1 | chr8 | 17922840 | 18027975 | + |
| PCSK7 | chr11 | 117204337 | 117232525 | − |
| PDCD1 | chr2 | 241849884 | 241858894 | − |
| PDCD11 | chr10 | 103396626 | 103446294 | + |
| PDCD1LG2 | chr9 | 5510531 | 5571282 | + |
| PDE4DIP | chr1 | 148808181 | 149048286 | + |
| PDGFB | chr22 | 39223359 | 39244982 | − |
| PDGFD | chr11 | 103907189 | 104164379 | − |
| PDGFRA | chr4 | 54229280 | 54298245 | + |
| PDGFRB | chr5 | 150113839 | 150155872 | − |
| PDK1 | chr2 | 172555373 | 172608669 | + |
| PDPK1 | chr16 | 2537979 | 2603188 | + |
| PDS5B | chr13 | 32586452 | 32778019 | + |
| PER1 | chr17 | 8140472 | 8156506 | − |
| PGAP3 | chr17 | 39671122 | 39696797 | − |
| PGBD5 | chr1 | 230314490 | 230426332 | − |
| PGLS | chr19 | 17511636 | 17521288 | + |
| PGR | chr11 | 101029624 | 101129813 | − |
| PHF1 | chr6 | 33410399 | 33416453 | + |
| PHF6 | chrX | 134373312 | 134428791 | + |
| PHKB | chr16 | 47461123 | 47701523 | + |
| PHLPP2 | chr16 | 71637835 | 71724701 | − |
| PHOX2B | chr4 | 41744082 | 41748725 | − |
| PICALM | chr11 | 85957175 | 86069882 | − |
| PIGA | chrX | 15319452 | 15335554 | − |
| PIK3C2B | chr1 | 204422628 | 204494805 | − |
| PIK3C2G | chr12 | 18242961 | 18648416 | + |
| PIK3C3 | chr18 | 41955234 | 42087830 | + |
| PIK3CA | chr3 | 179148114 | 179240093 | + |
| PIK3CB | chr3 | 138652698 | 138834928 | − |
| PIK3CD | chr1 | 9651731 | 9729114 | + |
| PIK3CG | chr7 | 106865278 | 106908980 | + |
| PIK3R1 | chr5 | 68215756 | 68301821 | + |
| PIK3R2 | chr19 | 18153163 | 18170532 | + |
| PIK3R3 | chr1 | 46040140 | 46133036 | − |
| PIM1 | chr6 | 37170152 | 37175428 | + |
| PIM2 | chrX | 48913182 | 48919024 | − |
| PKD1L2 | chr16 | 81100875 | 81220370 | − |
| PKHD1 | chr6 | 51615299 | 52087613 | − |
| PKN1 | chr19 | 14433053 | 14471867 | + |
| PLAG1 | chr8 | 56160909 | 56211324 | − |
| PLCG1 | chr20 | 41136960 | 41196801 | + |
| PLCG2 | chr16 | 81739097 | 81962685 | + |
| PLEKHG5 | chr1 | 6467122 | 6520074 | − |
| PLEKHG6 | chr12 | 6310436 | 6328506 | + |
| PLEKHS1 | chr10 | 113751262 | 113783429 | + |
| PLK2 | chr5 | 58453982 | 58460139 | − |
| PMAIP1 | chr18 | 59899996 | 59904305 | + |
| PML | chr15 | 73994673 | 74047827 | + |
| PMS1 | chr2 | 189784085 | 189877629 | + |
| PMS2 | chr7 | 5970925 | 6009106 | − |
| PNRC1 | chr6 | 89080751 | 89085160 | + |
| POLD1 | chr19 | 50384204 | 50418018 | + |
| POLE | chr12 | 132623753 | 132687376 | − |
| POLG | chr15 | 89305198 | 89334861 | − |
| POLQ | chr3 | 121431431 | 121545988 | − |
| POT1 | chr7 | 124822386 | 124929983 | − |
| POU2AF1 | chr11 | 111352255 | 111455630 | − |
| POU5F1 | chr6 | 31164337 | 31180731 | − |
| PPARG | chr3 | 12287368 | 12434356 | + |
| PPAT | chr4 | 56393362 | 56435615 | − |
| PPFIBP1 | chr12 | 27523431 | 27695564 | + |
| PPM1D | chr17 | 60600183 | 60666280 | + |
| PPP1CB | chr2 | 28751640 | 28802940 | + |
| PPP2R1A | chr19 | 52190048 | 52229518 | + |
| PPP2R2A | chr8 | 26291508 | 26372680 | + |
| PPP4R2 | chr3 | 72996803 | 73069198 | + |
| PPP6C | chr9 | 125146573 | 125189939 | − |
| PRCC | chr1 | 156750610 | 156800815 | + |
| PRDM1 | chr6 | 105993463 | 106109939 | + |
| PRDM10 | chr11 | 129899706 | 130002835 | − |
| PRDM14 | chr8 | 70051651 | 70071693 | − |
| PRDM16 | chr1 | 3069168 | 3438621 | + |
| PRDM2 | chr1 | 13700198 | 13825079 | + |
| PREX2 | chr8 | 67952046 | 68237032 | + |
| PRF1 | chr10 | 70597348 | 70602759 | − |
| PRIM1 | chr12 | 56731296 | 56752374 | − |
| PRKACA | chr19 | 14091688 | 14118084 | − |
| PRKACB | chr1 | 84078062 | 84238498 | + |
| PRKAR1A | chr17 | 68511780 | 68551319 | + |
| PRKAR2B | chr7 | 107044705 | 107161811 | + |
| PRKCA | chr17 | 66302613 | 66810743 | + |
| PRKCB | chr16 | 23835983 | 24220611 | + |
| PRKCD | chr3 | 53156009 | 53192717 | + |
| PRKCI | chr3 | 170222424 | 170305977 | + |
| PRKD1 | chr14 | 29576479 | 30191898 | − |
| PRKD2 | chr19 | 46674275 | 46717127 | − |
| PRKD3 | chr2 | 37250502 | 37324833 | − |
| PRKDC | chr8 | 47773111 | 47960178 | − |
| PRKN | chr6 | 161347417 | 162727775 | − |
| PRPF40B | chr12 | 49568218 | 49644666 | + |
| PRPF8 | chr17 | 1650629 | 1684867 | − |
| PRRX1 | chr1 | 170662728 | 170739421 | + |
| PRSS8 | chr16 | 31131433 | 31135727 | − |
| PSIP1 | chr9 | 15464066 | 15510995 | − |
| PTCH1 | chr9 | 95442980 | 95517057 | − |
| PTEN | chr10 | 87863625 | 87971930 | + |
| PTGDR | chr14 | 52267698 | 52276724 | + |
| PTGS2 | chr1 | 186671791 | 186680922 | − |
| PTK2B | chr8 | 27311482 | 27459391 | + |
| PTK6 | chr20 | 63528001 | 63537376 | − |
| PTK7 | chr6 | 43076307 | 43161719 | + |
| PTP4A1 | chr6 | 63521746 | 63583588 | + |
| PTPN1 | chr20 | 50510321 | 50585241 | + |
| PTPN11 | chr12 | 112418351 | 112509918 | + |
| PTPN13 | chr4 | 86594315 | 86815171 | + |
| PTPN2 | chr18 | 12785478 | 12929643 | − |
| PTPN6 | chr12 | 6946468 | 6961316 | + |
| PTPRB | chr12 | 70515870 | 70637440 | − |
| PTPRC | chr1 | 198638457 | 198757476 | + |
| PTPRD | chr9 | 8314246 | 10613002 | − |
| PTPRK | chr6 | 127968785 | 128520616 | − |
| PTPRO | chr12 | 15322257 | 15602175 | + |
| PTPRS | chr19 | 5158495 | 5340803 | − |
| PTPRT | chr20 | 42072752 | 43189970 | − |
| PWWP2A | chr5 | 160061801 | 160119450 | − |
| PYCR1 | chr17 | 81932384 | 81942412 | − |
| QKI | chr6 | 163414000 | 163578592 | + |
| RAB29 | chr1 | 205767986 | 205775482 | − |
| RAB35 | chr12 | 120095099 | 120117502 | − |
| RAB5C | chr17 | 42124978 | 42155044 | − |
| RABEP1 | chr17 | 5282265 | 5386340 | + |
| RAC1 | chr7 | 6374527 | 6403967 | + |
| RAC2 | chr22 | 37225270 | 37244448 | − |
| RAD17 | chr5 | 69369293 | 69414801 | + |
| RAD21 | chr8 | 116845934 | 116874776 | − |
| RAD50 | chr5 | 132556019 | 132646349 | + |
| RAD51 | chr15 | 40694774 | 40732340 | + |
| RAD51B | chr14 | 67819779 | 68730218 | + |
| RAD51C | chr17 | 58692573 | 58735611 | + |
| RAD51D | chr17 | 35092221 | 35121522 | − |
| RAD52 | chr12 | 911736 | 990053 | − |
| RAD54L | chr1 | 46246461 | 46278480 | + |
| RAF1 | chr3 | 12583601 | 12664125 | − |
| RAG1 | chr11 | 36510709 | 36593156 | + |
| RAG2 | chr11 | 36575574 | 36598279 | − |
| RALGDS | chr9 | 133097720 | 133149334 | − |
| RANBP1 | chr22 | 20115938 | 20127355 | + |
| RANBP2 | chr2 | 108719482 | 108785809 | + |
| RAP1GDS1 | chr4 | 98261384 | 98443858 | + |
| RARA | chr17 | 40309180 | 40357643 | + |
| RASA1 | chr5 | 87267883 | 87391931 | + |
| RASGEF1A | chr10 | 43194535 | 43267065 | − |
| RB1 | chr13 | 48303744 | 48599436 | + |
| RBBP6 | chr16 | 24537693 | 24572863 | + |
| RBM10 | chrX | 47145221 | 47186813 | + |
| RBM15 | chr1 | 110338506 | 110346681 | + |
| RECQL | chr12 | 21468910 | 21501669 | − |
| RECQL4 | chr8 | 144511288 | 144517845 | − |
| REL | chr2 | 60881521 | 60931612 | + |
| RELA | chr11 | 65653597 | 65663090 | − |
| RELN | chr7 | 103471381 | 103989658 | − |
| REST | chr4 | 56907876 | 56966808 | + |
| RET | chr10 | 43077064 | 43130351 | + |
| RFWD3 | chr16 | 74621399 | 74666877 | − |
| RGPD3 | chr2 | 106391290 | 106468413 | − |
| RGS7 | chr1 | 240775514 | 241357230 | − |
| RHEB | chr7 | 151466012 | 151520120 | − |
| RHOA | chr3 | 49359139 | 49412998 | − |
| RHOH | chr4 | 40191053 | 40246967 | + |
| RICTOR | chr5 | 38937920 | 39074399 | − |
| RIT1 | chr1 | 155897808 | 155911404 | − |
| RMI2 | chr16 | 11249619 | 11381662 | + |
| RNASEL | chr1 | 182573634 | 182589256 | − |
| RNF2 | chr1 | 185045526 | 185102603 | + |
| RNF213 | chr17 | 80260866 | 80398786 | + |
| RNF217-AS1 | chr6 | 124644434 | 124963165 | − |
| RNF43 | chr17 | 58353676 | 58417595 | − |
| ROBO1 | chr3 | 78597239 | 79767998 | − |
| ROBO2 | chr3 | 75906695 | 77649964 | + |
| ROS1 | chr6 | 117287353 | 117425942 | − |
| RPL10 | chrX | 154389955 | 154409168 | + |
| RPL22 | chr1 | 6185020 | 6209389 | − |
| RPL5 | chr1 | 92832013 | 92841924 | + |
| RPN1 | chr3 | 128619969 | 128681075 | − |
| RPS14 | chr5 | 150442635 | 150449739 | − |
| RPS15 | chr19 | 1438358 | 1440495 | + |
| RPS6KA2 | chr6 | 166409364 | 166906451 | − |
| RPS6KA4 | chr11 | 64359148 | 64372215 | + |
| RPS6KB2 | chr11 | 67428460 | 67435401 | + |
| RPS7 | chr2 | 3575260 | 3580920 | + |
| RPTOR | chr17 | 80544819 | 80966371 | + |
| RRAGC | chr1 | 38838198 | 38859772 | − |
| RRAS | chr19 | 49635292 | 49640143 | − |
| RRAS2 | chr11 | 14277922 | 14364506 | − |
| RRM1 | chr11 | 4094707 | 4138932 | + |
| RSPO2 | chr8 | 107899316 | 108083642 | − |
| RSPO3 | chr6 | 127118671 | 127199481 | + |
| RTEL1 | chr20 | 63657810 | 63696253 | + |
| RUNX1 | chr21 | 34787801 | 36004667 | − |
| RUNX1T1 | chr8 | 91954967 | 92103286 | − |
| RUNX2 | chr6 | 45328157 | 45664349 | + |
| RXRA | chr9 | 134317098 | 134440585 | + |
| RYBP | chr3 | 72371825 | 72446623 | − |
| S100A7 | chr1 | 153457744 | 153460651 | − |
| S1PR2 | chr19 | 10221433 | 10231331 | − |
| SALL4 | chr20 | 51782331 | 51802521 | − |
| SAMD9 | chr7 | 93099513 | 93118023 | − |
| SAMHD1 | chr20 | 36890229 | 36951893 | − |
| SBDS | chr7 | 66987680 | 66995587 | − |
| SCG5 | chr15 | 32641676 | 32697098 | + |
| SDC4 | chr20 | 45325288 | 45348424 | − |
| SDHA | chr5 | 218241 | 257082 | + |
| SDHAF2 | chr11 | 61430042 | 61446733 | + |
| SDHB | chr1 | 17018722 | 17054032 | − |
| SDHC | chr1 | 161314381 | 161363206 | + |
| SDHD | chr11 | 112086824 | 112120016 | + |
| SEC31A | chr4 | 82818509 | 82901166 | − |
| SEMA6A | chr5 | 116443555 | 116574823 | − |
| SEPTIN5 | chr22 | 19714503 | 19724224 | + |
| SEPTIN6 | chrX | 119615724 | 119693370 | − |
| SEPTIN9 | chr17 | 77280569 | 77500596 | + |
| SERP2 | chr13 | 44373665 | 44397714 | + |
| SERPINA9 | chr14 | 94462717 | 94479689 | − |
| SERPINB3 | chr18 | 63655197 | 63661893 | − |
| SERPINB4 | chr18 | 63637259 | 63644256 | − |
| SESN1 | chr6 | 108986437 | 109094819 | − |
| SESN2 | chr1 | 28259518 | 28282491 | + |
| SESN3 | chr11 | 95165513 | 95232541 | − |
| SET | chr9 | 128683424 | 128696400 | + |
| SETBP1 | chr18 | 44680173 | 45068510 | + |
| SETD1A | chr16 | 30957754 | 30984664 | + |
| SETD1B | chr12 | 121804009 | 121832656 | + |
| SETD2 | chr3 | 47016429 | 47164113 | − |
| SETD3 | chr14 | 99397748 | 99480889 | − |
| SETD4 | chr21 | 36034541 | 36079389 | − |
| SETD5 | chr3 | 9397615 | 9479240 | + |
| SETD6 | chr16 | 58515479 | 58523842 | + |
| SETD7 | chr4 | 139495941 | 139606699 | − |
| SETDB1 | chr1 | 150926263 | 150964744 | + |
| SETDB2 | chr13 | 49444374 | 49495003 | + |
| SF3B1 | chr2 | 197388515 | 197435079 | − |
| SFPQ | chr1 | 35176378 | 35193145 | − |
| SFRP1 | chr8 | 41261962 | 41309473 | − |
| SFRP2 | chr4 | 153780591 | 153789083 | − |
| SFRP4 | chr7 | 37905932 | 38025695 | − |
| SGK1 | chr6 | 134169248 | 134318112 | − |
| SH2B3 | chr12 | 111405923 | 111451623 | + |
| SH2D1A | chrX | 124227868 | 124373197 | + |
| SH3BP5 | chr3 | 15254353 | 15341368 | − |
| SH3GL1 | chr19 | 4360370 | 4400547 | − |
| SHOC2 | chr10 | 110919593 | 111013666 | + |
| SHQ1 | chr3 | 72749277 | 72861914 | − |
| SHTN1 | chr10 | 116881477 | 117126586 | − |
| SIRPA | chr20 | 1894167 | 1940592 | + |
| SIX1 | chr14 | 60643421 | 60658259 | − |
| SIX2 | chr2 | 45005182 | 45009452 | − |
| SKI | chr1 | 2228319 | 2310213 | + |
| SLC1A2 | chr11 | 35251205 | 35420063 | − |
| SLC29A1 | chr6 | 44219553 | 44234142 | + |
| SLC34A2 | chr4 | 25648011 | 25678748 | + |
| SLC45A3 | chr1 | 205657851 | 205680509 | − |
| SLFN11 | chr17 | 35350305 | 35373701 | − |
| SLX4 | chr16 | 3581181 | 3611606 | − |
| SMAD2 | chr18 | 47808957 | 47931146 | − |
| SMAD3 | chr15 | 67063763 | 67195173 | + |
| SMAD4 | chr18 | 51028394 | 51085045 | + |
| SMARCA1 | chrX | 129446501 | 129523500 | − |
| SMARCA2 | chr9 | 1980290 | 2193624 | + |
| SMARCA4 | chr19 | 10960932 | 11079426 | + |
| SMARCB1 | chr22 | 23786931 | 23838009 | + |
| SMARCD1 | chr12 | 50085200 | 50100707 | + |
| SMARCE1 | chr17 | 40624962 | 40648654 | − |
| SMC1A | chrX | 53374149 | 53422728 | − |
| SMC3 | chr10 | 110567695 | 110606048 | + |
| SMG1 | chr16 | 18804860 | 18926408 | − |
| SMO | chr7 | 129188633 | 129213545 | + |
| SMUG1 | chr12 | 54121277 | 54189008 | − |
| SMYD3 | chr1 | 245749342 | 246507312 | − |
| SNCAIP | chr5 | 122311354 | 122464219 | + |
| SND1 | chr7 | 127652194 | 128092609 | + |
| SNX29 | chr16 | 11976734 | 12574287 | + |
| SNX31 | chr8 | 100572889 | 100663415 | − |
| SOCS1 | chr16 | 11254417 | 11256204 | − |
| SOCS2 | chr12 | 93569814 | 93583487 | + |
| SOCS3 | chr17 | 78356778 | 78360077 | − |
| SOS1 | chr2 | 38981549 | 39124345 | − |
| SOX10 | chr22 | 37970686 | 37987422 | − |
| SOX11 | chr2 | 5692384 | 5701385 | + |
| SOX17 | chr8 | 54457935 | 54460892 | + |
| SOX2 | chr3 | 181711925 | 181714436 | + |
| SOX21 | chr13 | 94709622 | 94712545 | − |
| SOX9 | chr17 | 72121020 | 72126416 | + |
| SP140 | chr2 | 230203110 | 230313215 | + |
| SPECC1 | chr17 | 20008865 | 20319026 | + |
| SPEN | chr1 | 15836095 | 15940456 | + |
| SPI1 | chr11 | 47354860 | 47378547 | − |
| SPOP | chr17 | 49598884 | 49678163 | − |
| SPRED1 | chr15 | 38252836 | 38357249 | + |
| SPRTN | chr1 | 231337104 | 231355023 | + |
| SPTA1 | chr1 | 158610704 | 158686715 | − |
| SRC | chr20 | 37344685 | 37406050 | + |
| SRGAP3 | chr3 | 8980591 | 9363053 | − |
| SRSF2 | chr17 | 76734115 | 76737333 | − |
| SRSF3 | chr6 | 36594353 | 36605600 | + |
| SS18 | chr18 | 26016253 | 26091217 | − |
| SS18L1 | chr20 | 62143769 | 62182514 | + |
| SSH1 | chr12 | 108778191 | 108857590 | − |
| SSR3 | chr3 | 156539553 | 156555149 | − |
| SSX1 | chrX | 48255392 | 48267444 | + |
| SSX2 | chrX | 52696896 | 52707189 | − |
| SSX4 | chrX | 48383516 | 48393347 | + |
| STAG1 | chr3 | 136336236 | 136752403 | − |
| STAG2 | chrX | 123960212 | 124422664 | + |
| STAT1 | chr2 | 190908460 | 191020960 | − |
| STAT3 | chr17 | 42313324 | 42388540 | − |
| STAT4 | chr2 | 191029576 | 191151596 | − |
| STAT5A | chr17 | 42287547 | 42311943 | + |
| STAT5B | chr17 | 42199177 | 42276707 | − |
| STAT6 | chr12 | 57095408 | 57132139 | − |
| STIL | chr1 | 47250139 | 47314892 | − |
| STK11 | chr19 | 1177558 | 1228431 | + |
| STK19 | chr6 | 31971091 | 31982821 | + |
| STK36 | chr2 | 218672069 | 218702716 | + |
| STK40 | chr1 | 36339624 | 36385924 | − |
| STRBP | chr9 | 123109500 | 123268586 | − |
| STRN | chr2 | 36837698 | 36966536 | − |
| STX11 | chr6 | 144150517 | 144191939 | + |
| STX7 | chr6 | 132445867 | 132513198 | − |
| SUFU | chr10 | 102503972 | 102633535 | + |
| SUZ12 | chr17 | 31937007 | 32001038 | + |
| SYK | chr9 | 90801787 | 90898549 | + |
| SYNE1 | chr6 | 152121687 | 152637801 | − |
| SYT1 | chr12 | 78863993 | 79452008 | + |
| TAF1 | chrX | 71366222 | 71532374 | + |
| TAF15 | chr17 | 35713791 | 35864615 | + |
| TAF1L | chr9 | 32629454 | 32635669 | − |
| TAL1 | chr1 | 47216290 | 47232220 | − |
| TAL2 | chr9 | 105662457 | 105663124 | + |
| TAP1 | chr6 | 32845209 | 32853816 | − |
| TAP2 | chr6 | 32821833 | 32838770 | − |
| TBL1XR1 | chr3 | 177019340 | 177228000 | − |
| TBX22 | chrX | 80014753 | 80031774 | + |
| TBX3 | chr12 | 114670255 | 114684175 | − |
| TCEA1 | chr8 | 53966552 | 54022456 | − |
| TCF12 | chr15 | 56918623 | 57299281 | + |
| TCF3 | chr19 | 1609290 | 1652615 | − |
| TCF7L1 | chr2 | 85133392 | 85310387 | + |
| TCF7L2 | chr10 | 112950247 | 113167678 | + |
| TCL1A | chr14 | 95709947 | 95714196 | − |
| TCL1B | chr14 | 95686426 | 95692628 | + |
| TEC | chr4 | 48135783 | 48269838 | − |
| TEK | chr9 | 27109141 | 27230174 | + |
| TEKT4 | chr2 | 94871430 | 94876823 | + |
| TENT5C | chr1 | 117606048 | 117628389 | + |
| TERC | chr3 | 169764520 | 169765060 | − |
| TERT | chr5 | 1253147 | 1295068 | − |
| TET1 | chr10 | 68560337 | 68694487 | + |
| TET2 | chr4 | 105145875 | 105279816 | + |
| TET3 | chr2 | 73984910 | 74108176 | + |
| TFE3 | chrX | 49028726 | 49043410 | − |
| TFEB | chr6 | 41683978 | 41736259 | − |
| TFG | chr3 | 100709295 | 100748964 | + |
| TFPT | chr19 | 54107020 | 54115657 | − |
| TFRC | chr3 | 196027183 | 196082096 | − |
| TGFB1 | chr19 | 41301587 | 41353922 | − |
| TGFBR1 | chr9 | 99104038 | 99154192 | + |
| TGFBR2 | chr3 | 30606601 | 30694142 | + |
| TGFBR3 | chr1 | 91680343 | 91906335 | − |
| TGM7 | chr15 | 43276271 | 43302255 | − |
| THADA | chr2 | 43230851 | 43596038 | − |
| THBS1 | chr15 | 39581079 | 39599466 | + |
| THRAP3 | chr1 | 36224432 | 36305357 | + |
| TIMP3 | chr22 | 32801705 | 32863041 | + |
| TIPARP | chr3 | 156673235 | 156706770 | + |
| TLL2 | chr10 | 96364608 | 96513926 | − |
| TLR2 | chr4 | 153684070 | 153706260 | + |
| TLR4 | chr9 | 117704175 | 117724735 | + |
| TLX1 | chr10 | 101131300 | 101137789 | + |
| TLX3 | chr5 | 171309248 | 171312139 | + |
| TMEM127 | chr2 | 96248514 | 96265997 | − |
| TMEM216 | chr11 | 61392360 | 61398863 | + |
| TMEM30A | chr6 | 75252924 | 75284948 | − |
| TMPRSS2 | chr21 | 41464300 | 41531116 | − |
| TMSB4X | chrX | 12975110 | 12977227 | + |
| TMSB4XP8 | chr4 | 90838903 | 90839037 | − |
| TNC | chr9 | 115019575 | 115118257 | − |
| TNFAIP3 | chr6 | 137867214 | 137883312 | + |
| TNFRSF11A | chr18 | 62325287 | 62391288 | + |
| TNFRSF13B | chr17 | 16929816 | 16972118 | − |
| TNFRSF14 | chr1 | 2555639 | 2565382 | + |
| TNFRSF17 | chr16 | 11965210 | 11968068 | + |
| TNFRSF1B | chr1 | 12166991 | 12209228 | + |
| TNFSF4 | chr1 | 173183731 | 173207331 | − |
| TNK2 | chr3 | 195863364 | 195911945 | − |
| TOP1 | chr20 | 41028822 | 41124487 | + |
| TP53 | chr17 | 7661779 | 7687538 | − |
| TP53BP1 | chr15 | 43403061 | 43510728 | − |
| TP63 | chr3 | 189631389 | 189897276 | + |
| TPM3 | chr1 | 154155304 | 154194648 | − |
| TPM4 | chr19 | 16067021 | 16103002 | + |
| TPR | chr1 | 186311652 | 186375693 | − |
| TRAF2 | chr9 | 136881912 | 136926607 | + |
| TRAF3 | chr14 | 102777449 | 102911500 | + |
| TRAF5 | chr1 | 211326615 | 211374946 | + |
| TRAF7 | chr16 | 2155698 | 2178129 | + |
| TRHDE | chr12 | 72087266 | 72670758 | + |
| TRIM24 | chr7 | 138460259 | 138589996 | + |
| TRIM27 | chr6 | 28903002 | 28923988 | − |
| TRIM33 | chr1 | 114392790 | 114511203 | − |
| TRIP11 | chr14 | 91965991 | 92040896 | − |
| TRIP13 | chr5 | 892884 | 919357 | + |
| TRRAP | chr7 | 98877933 | 99050831 | + |
| TSC1 | chr9 | 132891348 | 132946874 | − |
| TSC2 | chr16 | 2047967 | 2089491 | + |
| TSHR | chr14 | 80954989 | 81146306 | + |
| TSPAN31 | chr12 | 57738013 | 57750219 | + |
| TTL | chr2 | 112482156 | 112541739 | + |
| TUSC3 | chr8 | 15417215 | 15766649 | + |
| TYK2 | chr19 | 10350529 | 10380572 | − |
| TYRO3 | chr15 | 41557675 | 41583589 | + |
| U2AF1 | chr21 | 43092956 | 43107570 | − |
| U2AF2 | chr19 | 55654146 | 55674716 | + |
| UBR5 | chr8 | 102252273 | 102412759 | − |
| UEVLD | chr11 | 18529609 | 18588747 | − |
| UGT1A1 | chr2 | 233760270 | 233773300 | + |
| UMODL1 | chr21 | 42062959 | 42143453 | + |
| UNC13A | chr19 | 17601336 | 17688365 | − |
| UPF1 | chr19 | 18831959 | 18868230 | + |
| USP44 | chr12 | 95516560 | 95551476 | − |
| USP6 | chr17 | 5116032 | 5175034 | + |
| USP8 | chr15 | 50424380 | 50514421 | + |
| USP9X | chrX | 41085445 | 41236579 | + |
| VAV1 | chr19 | 6772708 | 6857366 | + |
| VAV2 | chr9 | 133761894 | 133992604 | − |
| VEGFA | chr6 | 43770184 | 43786487 | + |
| VGLL2 | chr6 | 117265558 | 117273565 | + |
| VGLL3 | chr3 | 86876388 | 86991149 | − |
| VHL | chr3 | 10141778 | 10153667 | + |
| VTCN1 | chr1 | 117143587 | 117210960 | − |
| VTI1A | chr10 | 112446998 | 112818744 | + |
| WAS | chrX | 48676596 | 48691427 | + |
| WDCP | chr2 | 24029347 | 24049575 | − |
| WDR62 | chr19 | 36054649 | 36105108 | + |
| WDR90 | chr16 | 649311 | 667833 | + |
| WIF1 | chr12 | 65050626 | 65121305 | − |
| WNK2 | chr9 | 93184916 | 93320572 | + |
| WRN | chr8 | 31033788 | 31176138 | + |
| WT1 | chr11 | 32387775 | 32435564 | − |
| WWP1 | chr8 | 86342547 | 86478420 | + |
| WWTR1 | chr3 | 149517235 | 149736714 | − |
| XBP1 | chr22 | 28794555 | 28800597 | − |
| XIAP | chrX | 123859724 | 123913976 | + |
| XKR4 | chr8 | 55102028 | 55542054 | + |
| XPA | chr9 | 97674909 | 97697340 | − |
| XPC | chr3 | 14145147 | 14178621 | − |
| XPO1 | chr2 | 61476032 | 61538741 | − |
| XRCC2 | chr7 | 152644776 | 152676141 | − |
| YAF2 | chr12 | 42157104 | 42238349 | − |
| YAP1 | chr11 | 102110447 | 102233424 | + |
| YES1 | chr18 | 721588 | 812546 | − |
| YPEL5 | chr2 | 30146941 | 30160533 | + |
| YWHAE | chr17 | 1344275 | 1400222 | − |
| YY1 | chr14 | 100238298 | 100282788 | + |
| YY1AP1 | chr1 | 155659443 | 155689000 | − |
| ZBTB16 | chr11 | 114059041 | 114256765 | + |
| ZBTB20 | chr3 | 114314500 | 115147288 | − |
| ZBTB7A | chr19 | 4043303 | 4066899 | − |
| ZCCHC7 | chr9 | 37120574 | 37358149 | + |
| ZCCHC8 | chr12 | 122471599 | 122501073 | − |
| ZEB1 | chr10 | 31318495 | 31529814 | + |
| ZFHX3 | chr16 | 72782885 | 73891871 | − |
| ZFP36L1 | chr14 | 68787660 | 68796253 | − |
| ZMYM2 | chr13 | 19958677 | 20091829 | + |
| ZMYM3 | chrX | 71239624 | 71255146 | − |
| ZNF217 | chr20 | 53567071 | 53609907 | − |
| ZNF24 | chr18 | 35332227 | 35345482 | − |
| ZNF318 | chr6 | 43307134 | 43369647 | − |
| ZNF331 | chr19 | 53519527 | 53580269 | + |
| ZNF384 | chr12 | 6666477 | 6689572 | − |
| ZNF429 | chr19 | 21496682 | 21556270 | + |
| ZNF443 | chr19 | 12429706 | 12441021 | − |
| ZNF479 | chr7 | 57117676 | 57139864 | − |
| ZNF521 | chr18 | 25061924 | 25352190 | − |
| ZNF526 | chr19 | 42220312 | 42228201 | + |
| ZNF544 | chr19 | 58228594 | 58277495 | + |
| ZNF559 | chr19 | 9323772 | 9351162 | + |
| ZNF638 | chr2 | 71276561 | 71435069 | + |
| ZNF703 | chr8 | 37695782 | 37700019 | + |
| ZNF750 | chr17 | 82829434 | 82840022 | − |
| ZNRF3 | chr22 | 28883572 | 29057488 | + |
| ZRSR2 | chrX | 15790472 | 15823260 | + |
| ZSWIM4 | chr19 | 13795460 | 13832230 | + |

APPENDIX 2

| Cancer | | | | |
|---|---|---|---|---|
| gene name | Chromosome | Gene Start | Gene End | Strand |
| ABL1 | chr9 | 130713016 | 130887675 | + |
| ALK | chr2 | 29192774 | 29921586 | − |
| ATM | chr11 | 108223044 | 108369102 | + |
| BCR | chr22 | 23179704 | 23318037 | + |
| BRAF | chr7 | 140719327 | 140924929 | − |
| BRCA1 | chr17 | 43044295 | 43170245 | − |
| BRCA2 | chr13 | 32315086 | 32400268 | + |
| BTK | chrX | 101349447 | 101390796 | − |
| CD274 | chr9 | 5450503 | 5470566 | + |
| CHEK1 | chr11 | 125625163 | 125676255 | + |
| CHEK2 | chr22 | 28687743 | 28742422 | − |
| EGFR | chr7 | 55019017 | 55211628 | + |
| ESR1 | chr6 | 151656691 | 152129619 | + |
| ETV1 | chr7 | 13891229 | 13991425 | − |
| ETV4 | chr17 | 43527844 | 43579620 | − |
| ETV6 | chr12 | 11649674 | 11895377 | + |
| EZH2 | chr7 | 148807257 | 148884321 | − |
| FGFR1 | chr8 | 38400215 | 38468834 | − |
| FGFR2 | chr10 | 121478332 | 121598458 | − |
| FGFR3 | chr4 | 1793293 | 1808872 | + |
| FOXO1 | chr13 | 40555667 | 40666641 | − |
| HRAS | chr11 | 532242 | 537321 | − |
| IDH1 | chr2 | 208236229 | 208266074 | − |
| IDH2 | chr15 | 90083045 | 90102477 | − |
| KRAS | chr12 | 25205246 | 25250936 | − |
| MAP2K1 | chr15 | 66386837 | 66491544 | + |
| MAP2K2 | chr19 | 4090321 | 4124122 | − |
| MDM2 | chr12 | 68808177 | 68845544 | + |
| MET | chr7 | 116672196 | 116798377 | + |
| MYBL1 | chr8 | 66562175 | 66614247 | − |
| MYC | chr8 | 127735434 | 127742951 | + |
| NRAS | chr1 | 114704469 | 114716771 | − |
| NRG1 | chr8 | 31639222 | 32855666 | + |
| NTRK1 | chr1 | 156815640 | 156881850 | + |
| NTRK2 | chr9 | 84668551 | 85027054 | + |
| NTRK3 | chr15 | 87859751 | 88256768 | − |
| PDCD1 | chr2 | 241849884 | 241858894 | − |
| PDGFRA | chr4 | 54229280 | 54298245 | + |
| PDGFRB | chr5 | 150113839 | 150155872 | − |
| PIK3CA | chr3 | 179148114 | 179240093 | + |
| PLAG1 | chr8 | 56160909 | 56211324 | − |
| RAD51B | chr14 | 67819779 | 68730218 | + |
| RAD51C | chr17 | 58692573 | 58735611 | + |
| RAD51D | chr17 | 35092221 | 35121522 | − |
| RAD54L | chr1 | 46246461 | 46278480 | + |
| RET | chr10 | 43077064 | 43130351 | + |
| ROS1 | chr6 | 117287353 | 117425942 | − |
| TERT | chr5 | 1253147 | 1295068 | − |
| TIGIT | chr3 | 114276913 | 114310288 | + |
| TMPRSS2 | chr21 | 41464300 | 41531116 | − |
| AKT1 | chr14 | 104769349 | 104795751 | − |
| AR | chrX | 67544021 | 67730619 | + |
| ARAF | chrX | 47561205 | 47571908 | + |
| ATR | chr3 | 142449007 | 142578733 | − |
| AURKA | chr20 | 56369389 | 56392337 | − |
| BAP1 | chr3 | 52401008 | 52410008 | − |
| BARD1 | chr2 | 214725646 | 214809683 | − |
| BCL2 | chr18 | 63123346 | 63320128 | − |
| BCOR | chrX | 40049815 | 40177329 | − |
| BRIP1 | chr17 | 61679139 | 61863559 | − |
| CAMTA1 | chr1 | 6785454 | 7769706 | + |
| CCNB3 | chrX | 50202713 | 50351914 | + |
| CDK12 | chr17 | 39461486 | 39564907 | + |
| CDK4 | chr12 | 57747727 | 57756013 | − |
| CDK6 | chr7 | 92604921 | 92836573 | − |
| CDKN2A | chr9 | 21967752 | 21995301 | − |
| CIC | chr19 | 42268537 | 42295797 | + |
| CLDN18 | chr3 | 137998735 | 138033655 | + |
| CSF1 | chr1 | 109910242 | 109930992 | + |
| CSF1R | chr5 | 150053291 | 150113372 | − |
| EPC1 | chr10 | 32267751 | 32378798 | − |
| ERBB2 | chr17 | 39687914 | 39730426 | + |
| ERCC2 | chr19 | 45349837 | 45370918 | − |
| ERG | chr21 | 38380027 | 38661780 | − |
| EWSR1 | chr22 | 29268009 | 29300525 | + |
| FANCL | chr2 | 58159243 | 58241372 | − |
| FGFR4 | chr5 | 177086905 | 177098144 | + |
| FH | chr1 | 241497511 | 241519799 | − |
| FLCN | chr17 | 17212212 | 17237188 | − |
| FLI1 | chr11 | 128686535 | 128813267 | + |
| FLT3 | chr13 | 28003274 | 28100592 | − |
| FOSB | chr19 | 45467995 | 45475179 | + |
| FUS | chr16 | 31180138 | 31191605 | + |
| GLI1 | chr12 | 57459785 | 57472268 | + |
| HMGA2 | chr12 | 65824460 | 65966291 | + |
| JAK2 | chr9 | 4984390 | 5129948 | + |
| JAZF1 | chr7 | 27830573 | 28180795 | − |
| KIT | chr4 | 54657918 | 54740715 | + |
| KMT2A | chr11 | 118436490 | 118526832 | + |
| MAPK1 | chr22 | 21759657 | 21867680 | − |
| MEAF6 | chr1 | 37489993 | 37514766 | − |
| MGMT | chr10 | 129467190 | 129770983 | + |
| MLH1 | chr3 | 36993350 | 37050846 | + |
| MSH2 | chr2 | 47403067 | 47663146 | + |
| MSH6 | chr2 | 47695530 | 47810063 | + |
| MTOR | chr1 | 11106535 | 11262551 | − |
| MYB | chr6 | 135181308 | 135219173 | + |
| NCOA2 | chr8 | 70109782 | 70403808 | − |
| NCOA3 | chr20 | 47501887 | 47656877 | + |
| NF1 | chr17 | 31094927 | 31382116 | + |
| NPM1 | chr5 | 171387116 | 171411810 | + |
| NUTM1 | chr15 | 34343315 | 34357737 | + |
| NUTM2A | chr10 | 87225448 | 87236908 | + |
| PALB2 | chr16 | 23603160 | 23641310 | − |
| PAX3 | chr2 | 222199887 | 222298998 | − |
| PAX7 | chr1 | 18630846 | 18748866 | + |
| PDGFB | chr22 | 39223359 | 39244982 | − |
| PGR | chr11 | 101029624 | 101129813 | − |
| PHF1 | chr6 | 33410399 | 33416453 | + |
| PMS2 | chr7 | 5970925 | 6009106 | − |
| PTCH1 | chr9 | 95442980 | 95517057 | − |
| PTEN | chr10 | 87863625 | 87971930 | + |
| RAD51 | chr15 | 40694774 | 40732340 | + |
| RAF1 | chr3 | 12583601 | 12664125 | − |
| RELA | chr11 | 65653597 | 65663090 | − |
| SDHA | chr5 | 218241 | 257082 | + |
| SDHB | chr1 | 17018722 | 17054032 | − |
| SDHC | chr1 | 161314381 | 161363206 | + |
| SDHD | chr11 | 112086824 | 112120016 | + |
| SMARCB1 | chr22 | 23786931 | 23838009 | + |
| SS18 | chr18 | 26016253 | 26091217 | − |
| STAT6 | chr12 | 57095408 | 57132139 | − |
| TAF15 | chr17 | 35713791 | 35864615 | + |
| TCF12 | chr15 | 56918623 | 57299281 | + |
| TFE3 | chrX | 49028726 | 49043410 | − |
| TFG | chr3 | 100709295 | 100748964 | + |
| THBS1 | chr15 | 39581079 | 39599466 | + |
| TSC1 | chr9 | 132891348 | 132946874 | − |
| TSC2 | chr16 | 2047967 | 2089491 | + |
| USP6 | chr17 | 5116032 | 5175034 | + |
| VEGFA | chr6 | 43770184 | 43786487 | + |
| VHL | chr3 | 10141778 | 10153667 | + |
| YAP1 | chr11 | 102110447 | 102233424 | + |
| YWHAE | chr17 | 1344275 | 1400222 | − |

What is claimed is:

1. A method for preparing a dual library of template nucleic acid to obtain sequence information from nucleic acid in a sample, said method comprising:
(I) fragmenting nucleic acid from a sample, thereby producing nucleic acid fragments;
(II) adding an affinity purification marker to ends of the nucleic acid fragments;
(III) ligating the ends of the nucleic acid fragments;
(IV) purifying nucleic acid from the sample;
(V) separating nucleic acid fragments with the affinity purification marker and nucleic acid fragments without the affinity purification marker; and
(VI) preparing both the nucleic acid fragments with the affinity purification marker and the nucleic acid fragments without the affinity purification marker for sequencing;
thereby creating a dual library of template nucleic acid.

2. The method of claim 1, comprising fragmenting the purified nucleic acid of step (IV).

3. The method of claim 1, comprising sequencing the dual library of template nucleic acid.

4. The method of claim 1, where step (VI) comprises (a) repairing ends of both (i) nucleic acid fragments with the affinity purification marker and (ii) nucleic acid fragments without the affinity purification marker; and
(b) ligating adaptors to both (i) nucleic acid fragments with the affinity purification marker and (ii) nucleic acid fragments without the affinity purification marker.

5. The method of claim 1, comprising (a) crosslinking the sample before the fragmenting of step (I) and (b) reversing the crosslinking after step (III).

6. The method of claim 4, comprising amplifying (i) nucleic acid fragments with the affinity purification marker and (ii) nucleic acid fragments without the affinity purification marker after step (b).

7. The method of claim 6, comprising separating the amplified nucleic acid fragments of (i) and unamplified template nucleic acid fragments with the affinity purification marker of (i).

8. The method of claim 6, comprising enriching for amplified nucleic acid fragments containing a target sequence by contacting the amplified nucleic acid fragments with an oligonucleotide capable of hybridizing to the target sequence.

9. The method of claim 8, comprising separating (a) amplified nucleic acid fragments hybridized to an oligonucleotide and (b) amplified nucleic acid fragments not hybridized to an oligonucleotide.

10. The method of claim 9, comprising (re) amplifying the amplified nucleic acid fragments hybridized to an oligonucleotide from part (a).

11. The method of claim 5, comprising enriching for amplified nucleic acid fragments without the affinity purification marker containing a target sequence by contacting the amplified nucleic acid fragments without the affinity purification marker with an oligonucleotide capable of hybridizing to the target sequence.

12. The method of claim 11, comprising separating (a) amplified nucleic acid fragments hybridized to an oligonucleotide and (b) amplified nucleic acid fragments not hybridized to an oligonucleotide.

13. The method of claim 12, comprising (re) amplifying the amplified nucleic acid fragments hybridized to an oligonucleotide from part (a).

14. The method of claim 10 or 13, comprising sequencing the (re) amplified nucleic acid fragments.

15. A method of analyzing a sample, said method comprising:
the method of claims 1-14, further comprising analyzing sequencing data derived from the dual library to determine the presence of absence of genetic variations in the sample.

16. A method for comprehensive genomic profiling of a sample, said method comprising:
the method of claims 1-14, further comprising analyzing sequencing data derived from the dual library to characterize genetic variations in the sample.

17. A kit comprising:
a restriction enzyme, a label, a ligase, a substrate capable of binding the label, end-repair reagents, a first set of adaptor oligonucleotides, a second set of adaptor oligonucleotides, a first set of target enrichment oligonucleotides, and a second set of target enrichment oligonucleotides.
